SEQUENCE LISTING (187 sequences)

SEQ ID NO: 1 - protein, 5 aa, synthetic construct
  REGION 1..5: note = Synthetic: 3C2 VH CDR1
GYYWS

SEQ ID NO: 2 - protein, 16 aa, synthetic construct
  REGION 1..16: note = Synthetic: 3C2 VH CDR2
YNYYSGSTNYNPSLKS

SEQ ID NO: 3 - protein, 10 aa, synthetic construct
  REGION 1..10: note = Synthetic: 3C2 VH CDR3
YPLIRGAFDY

SEQ ID NO: 4 - protein, 16 aa, synthetic construct
  REGION 1..16: note = Synthetic: 3C2 VL CDR1
RSSQNLLHTNGYNYLD

SEQ ID NO: 5 - protein, 7 aa, synthetic construct
  REGION 1..7: note = Synthetic: 3C2 VL CDR2
LGSNRAS

SEQ ID NO: 6 - protein, 9 aa, synthetic construct
  REGION 1..9: note = Synthetic: 3C2 VL CDR3
MQALQTPLT

SEQ ID NO: 7 - protein, 5 aa, synthetic construct
  REGION 1..5: note = Synthetic: 2B3 VH CDR1
GYYIH

SEQ ID NO: 8 - protein, 17 aa, synthetic construct
  REGION 1..17: note = Synthetic: 2B3 VH CDR2
WINPNSGGTNSAQKFQD

SEQ ID NO: 9 - protein, 18 aa, synthetic construct
  REGION 1..18: note = Synthetic: 2B3 VH CDR3
DRLVLPWFGEIFPDAFDI

SEQ ID NO: 10 - protein, 11 aa, synthetic construct
  REGION 1..11: note = Synthetic: 2B3 VL CDR1
RASQSIRSNLA

SEQ ID NO: 11 - protein, 7 aa, synthetic construct
  REGION 1..7: note = Synthetic: 2B3 VL CDR2
GASTRAT

SEQ ID NO: 12 - protein, 9 aa, synthetic construct
  REGION 1..9: note = Synthetic: 2B3 VL CDR3
QQYNNWPLT

SEQ ID NO: 13 - protein, 137 aa, synthetic construct
  REGION 1..137: note = Synthetic: 3C2 VH with signal sequence
MKHLWFCLLLVAAPRWVLSQAQLQESGPGLVKPSETLSLTCTVSTGSISGYYWSWIRQPPGKGLEWIGYNYYSGSTNYNPSLKSRVTISIDTSKNQFSLKLNSVTAADTAVYYCARYPLIRGAFDYWGQGTLVTVSS

SEQ ID NO: 14 - protein, 132 aa, synthetic construct
  REGION 1..132: note = Synthetic: 3C2 VL with signal sequence
MRLPAQLLGLLMLWVSGSSGDIVMTQSPLSLPVTPGEPASISCRSSQNLLHTNGYNYLDWYLQKPGQSPQLLIYLGSNRASGVPDRFSGSGSGTDFTLKISRVEAEDVGVYYCMQALQTPLTFGGGTKVEIK

SEQ ID NO: 15 - protein, 146 aa, synthetic construct
  REGION 1..146: note = Synthetic: 2B3 VH with signal sequence
MDWTWRILFLVAAATGAHSQVQLVQSGAEVKKPGASVKVSCKASGYTFTGYYIHWVRQAPGQGLEWMGWINPNSGGTNSAQKFQDRVTITRVTSINTAYMELSRLRSDDTAVYFCARDRLVLPWFGEIFPDAFDIWGQGTLVTVSS

SEQ ID NO: 16 - protein, 127 aa, synthetic construct
  REGION 1..127: note = Synthetic: 2B3 VL with signal sequence
MEAPAQLLFLLLLWLPDSTGEIVMTQSPATLSVSPGERATLSCRASQSIRSNLAWYQQKPGQAPRLLIYGASTRATGIPARFSGSGSGTEFTLTISSLQSENFAVYYCQQYNNWPLTFGGGTKVEIK

SEQ ID NO: 17 - protein, 118 aa, synthetic construct
  REGION 1..118: note = Synthetic: 3C2 VH without signal sequence
QAQLQESGPGLVKPSETLSLTCTVSTGSISGYYWSWIRQPPGKGLEWIGYNYYSGSTNYNPSLKSRVTISIDTSKNQFSLKLNSVTAADTAVYYCARYPLIRGAFDYWGQGTLVTVSS

SEQ ID NO: 18 - protein, 112 aa, synthetic construct
  REGION 1..112: note = Synthetic: 3C2 VL without signal sequence
DIVMTQSPLSLPVTPGEPASISCRSSQNLLHTNGYNYLDWYLQKPGQSPQLLIYLGSNRASGVPDRFSGSGSGTDFTLKISRVEAEDVGVYYCMQALQTPLTFGGGTKVEIK

SEQ ID NO: 19 - protein, 127 aa, synthetic construct
  REGION 1..127: note = Synthetic: 2B3 VH without signal sequence
QVQLVQSGAEVKKPGASVKVSCKASGYTFTGYYIHWVRQAPGQGLEWMGWINPNSGGTNSAQKFQDRVTITRVTSINTAYMELSRLRSDDTAVYFCARDRLVLPWFGEIFPDAFDIWGQGTLVTVSS

SEQ ID NO: 20 - protein, 107 aa, synthetic construct
  REGION 1..107: note = Synthetic: 2B3 VL without signal sequence
EIVMTQSPATLSVSPGERATLSCRASQSIRSNLAWYQQKPGQAPRLLIYGASTRATGIPARFSGSGSGTEFTLTISSLQSENFAVYYCQQYNNWPLTFGGGTKVEIK

SEQ ID NO: 21 - DNA, 411 bp, synthetic construct
  misc_feature 1..411: note = Synthetic: 3C2 VH DNA Sequence with signal sequence
  source 1..411: mol_type = other DNA
atgaaacatctgtggttctgccttctcctggtggcagctcccagatgggtcctgtcccaggcgcagctgcaggagtcgggcccaggactggtgaagccttcggagaccctgtccctcacctgcactgtctctactggctccatcagtggttactactggagctggatccggcagcccccagggaagggactggagtggattgggtataattattacagtgggagcaccaactacaacccctccctcaagagtcgagtcaccatatcaatagacacgtccaagaaccagttctccctgaagctgaattctgtgaccgctgcggacacggccgtatattactgtgcgagatatcctctgattcggggagcttttgactactggggccagggaaccctggtcaccgtctcctca

SEQ ID NO: 22 - DNA, 396 bp, synthetic construct
  misc_feature 1..396: note = Synthetic: 3C2 VL DNA Sequence with signal sequence
  source 1..396: mol_type = other DNA
atgaggctccctgctcagctcctggggctgctaatgctctgggtctctggatccagtggggatattgtgatgactcagtctccactctccctgcccgtcacccctggagagccggcctccatctcctgtaggtctagtcagaacctcctgcatactaatggctacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 23 - DNA, 438 bp, synthetic construct
  misc_feature 1..438: note = Synthetic: 2B3 VH DNA Sequence with signal sequence
  source 1..438: mol_type = other DNA
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaaccctaacagtggtggcacaaactctgcacagaagtttcaggacagggtcaccatcaccagggtcacgtccatcaacacagcctacatggagctgagcagactgagatctgacgacacggccgtgtatttctgtgcgagagatcggctcgtattaccatggttcggggaaatattcccagatgcttttgatatctggggccaagggacattggtcaccgtctcttca

SEQ ID NO: 24 - DNA, 381 bp, synthetic construct
  misc_feature 1..381: note = Synthetic: 2B3 VL DNA Sequence with signal sequence
  source 1..381: mol_type = other DNA
atggaagccccagcgcagcttctcttcctcctgctactctggctcccagattccactggagaaatagtgatgacgcagtctccagccaccctgtctgtgtctccaggggaaagagccaccctctcctgcagggccagtcagagtattaggagcaacttagcctggtatcagcagaaacctggccaggctcccaggctcctcatctatggtgcatccaccagggccactggtatcccagccaggttcagtggcagtgggtctgggacagagttcactctcaccatcagcagcctgcagtctgaaaattttgcagtttattactgtcagcagtataataactggcctctcactttcggcggagggaccaaggtggagatcaaa

SEQ ID NO: 25 - DNA, 354 bp, synthetic construct
  misc_feature 1..354: note = Synthetic: 3C2 VH DNA Sequence without signal sequence
  source 1..354: mol_type = other DNA
caggcgcagctgcaggagtcgggcccaggactggtgaagccttcggagaccctgtccctcacctgcactgtctctactggctccatcagtggttactactggagctggatccggcagcccccagggaagggactggagtggattgggtataattattacagtgggagcaccaactacaacccctccctcaagagtcgagtcaccatatcaatagacacgtccaagaaccagttctccctgaagctgaattctgtgaccgctgcggacacggccgtatattactgtgcgagatatcctctgattcggggagcttttgactactggggccagggaaccctggtcaccgtctcctca

SEQ ID NO: 26 - DNA, 336 bp, synthetic construct
  misc_feature 1..336: note = Synthetic: 3C2 VL DNA Sequence without signal sequence
  source 1..336: mol_type = other DNA
gatattgtgatgactcagtctccactctccctgcccgtcacccctggagagccggcctccatctcctgtaggtctagtcagaacctcctgcatactaatggctacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 27 - DNA, 381 bp, synthetic construct
  misc_feature 1..381: note = Synthetic: 2B3 VH DNA Sequence without signal sequence
  source 1..381: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaaccctaacagtggtggcacaaactctgcacagaagtttcaggacagggtcaccatcaccagggtcacgtccatcaacacagcctacatggagctgagcagactgagatctgacgacacggccgtgtatttctgtgcgagagatcggctcgtattaccatggttcggggaaatattcccagatgcttttgatatctggggccaagggacattggtcaccgtctcttca

SEQ ID NO: 28 - DNA, 321 bp, synthetic construct
  misc_feature 1..321: note = Synthetic: 2B3 VL DNA Sequence without signal sequence
  source 1..321: mol_type = other DNA
gaaatagtgatgacgcagtctccagccaccctgtctgtgtctccaggggaaagagccaccctctcctgcagggccagtcagagtattaggagcaacttagcctggtatcagcagaaacctggccaggctcccaggctcctcatctatggtgcatccaccagggccactggtatcccagccaggttcagtggcagtgggtctgggacagagttcactctcaccatcagcagcctgcagtctgaaaattttgcagtttattactgtcagcagtataataactggcctctcactttcggcggagggaccaaggtggagatcaaa

SEQ ID NO: 29 - protein, 5 aa, synthetic construct
  REGION 1..5: note = Synthetic: 7H7 VH CDR1
TSWMS

SEQ ID NO: 30 - protein, 17 aa, synthetic construct
  REGION 1..17: note = Synthetic: 7H7 VH CDR2
NIKQDGSEKYYVDSVKG

SEQ ID NO: 31 - protein, 10 aa, synthetic construct
  REGION 1..10: note = Synthetic: 7H7 VH CDR3
DRPVAGASAL

SEQ ID NO: 32 - protein, 11 aa, synthetic construct
  REGION 1..11: note = Synthetic: 7H7 VL CDR1
RASQSISGWLA

SEQ ID NO: 33 - protein, 7 aa, synthetic construct
  REGION 1..7: note = Synthetic: 7H7 VL CDR2
KASSLES

SEQ ID NO: 34 - protein, 9 aa, synthetic construct
  REGION 1..9: note = Synthetic: 7H7 VL CDR3
QQYYGSSRT

SEQ ID NO: 35 - protein, 5 aa, synthetic construct
  REGION 1..5: note = Synthetic: 1B3 VH CDR1
TSWMS

SEQ ID NO: 36 - protein, 17 aa, synthetic construct
  REGION 1..17: note = Synthetic: 1B3 VH CDR2
NIKQDGSEKYYVDSVKG

SEQ ID NO: 37 - protein, 10 aa, synthetic construct
  REGION 1..10: note = Synthetic: 1B3 VH CDR3
DRPVAGASAL

SEQ ID NO: 38 - protein, 11 aa, synthetic construct
  REGION 1..11: note = Synthetic: 1B3 VL CDR1
RASQSISGWLA

SEQ ID NO: 39 - protein, 7 aa, synthetic construct
  REGION 1..7: note = Synthetic: 1B3 VL CDR2
KASSLES

SEQ ID NO: 40 - protein, 9 aa, synthetic construct
  REGION 1..9: note = Synthetic: 1B3 VL CDR3
QQYYGSSRT

SEQ ID NO: 41 - protein, 5 aa, synthetic construct
  REGION 1..5: note = Synthetic: 3B6 VH CDR1
TYWMS

SEQ ID NO: 42 - protein, 17 aa, synthetic construct
  REGION 1..17: note = Synthetic: 3B6 VH CDR2
NIKQDGSEKYYVDSVKG

SEQ ID NO: 43 - protein, 10 aa, synthetic construct
  REGION 1..10: note = Synthetic: 3B6 VH CDR3
DRPVAGASAL

SEQ ID NO: 44 - protein, 11 aa, synthetic construct
  REGION 1..11: note = Synthetic: 3B6 VL CDR1
RASQSISGWLA

SEQ ID NO: 45 - protein, 7 aa, synthetic construct
  REGION 1..7: note = Synthetic: 3B6 VL CDR2
KASSLES

SEQ ID NO: 46 - protein, 9 aa, synthetic construct
  REGION 1..9: note = Synthetic: 3B6 VL CDR3
QQYYGSSRT

SEQ ID NO: 47 - protein, 5 aa, synthetic construct
  REGION 1..5: note = Synthetic: 8B1 VH CDR1
THWMS

SEQ ID NO: 48 - protein, 17 aa, synthetic construct
  REGION 1..17: note = Synthetic: 8B1 VH CDR2
NIKQDGSEKYYVDSVKG

SEQ ID NO: 49 - protein, 10 aa, synthetic construct
  REGION 1..10: note = Synthetic: 8B1 VH CDR3
DRPVAGASAL

SEQ ID NO: 50 - protein, 11 aa, synthetic construct
  REGION 1..11: note = Synthetic: 8B1 VL CDR1
RASQSISGWLA

SEQ ID NO: 51 - protein, 7 aa, synthetic construct
  REGION 1..7: note = Synthetic: 8B1 VL CDR2
KASSLES

SEQ ID NO: 52 - protein, 9 aa, synthetic construct
  REGION 1..9: note = Synthetic: 8B1 VL CDR3
QQYYGSSRT

SEQ ID NO: 53 - protein, 5 aa, synthetic construct
  REGION 1..5: note = Synthetic: 4A3 VH CDR1
SSWMS

SEQ ID NO: 54 - protein, 17 aa, synthetic construct
  REGION 1..17: note = Synthetic: 4A3 VH CDR2
NIKQDGSEKYYVDSVKG

SEQ ID NO: 55 - protein, 10 aa, synthetic construct
  REGION 1..10: note = Synthetic: 4A3 VH CDR3
DRPVAGASAL

SEQ ID NO: 56 - protein, 11 aa, synthetic construct
  REGION 1..11: note = Synthetic: 4A3 VL CDR1
RASQSISGWLA

SEQ ID NO: 57 - protein, 7 aa, synthetic construct
  REGION 1..7: note = Synthetic: 4A3 VL CDR2
KASSLES

SEQ ID NO: 58 - protein, 9 aa, synthetic construct
  REGION 1..9: note = Synthetic: 4A3 VL CDR3
QQYYGSSRT

SEQ ID NO: 59 - protein, 5 aa, synthetic construct
  REGION 1..5: note = Synthetic: 9H9 VH CDR1
TYWMS

SEQ ID NO: 60 - protein, 17 aa, synthetic construct
  REGION 1..17: note = Synthetic: 9H9 VH CDR2
NIKQDGSEKYYVDSVKG

SEQ ID NO: 61 - protein, 10 aa, synthetic construct
  REGION 1..10: note = Synthetic: 9H9 VH CDR3
DRPVAGASAL

SEQ ID NO: 62 - protein, 11 aa, synthetic construct
  REGION 1..11: note = Synthetic: 9H9 VL CDR1
RASQSISGWLA

SEQ ID NO: 63 - protein, 7 aa, synthetic construct
  REGION 1..7: note = Synthetic: 9H9 VL CDR2
KASSLES

SEQ ID NO: 64 - protein, 9 aa, synthetic construct
  REGION 1..9: note = Synthetic: 9H9 VL CDR3
QQYYGSSRT

SEQ ID NO: 65 - protein, 138 aa, synthetic construct
  REGION 1..138: note = Synthetic: 7H7 VH with signal sequence
  VARIANT 7: note = Xaa can be any naturally occurring amino acid
MELGLSXVFLVAILEGVQCEVQLVESGGGLVQPGGSLRLSCAASGGTISTSWMSWVRQAPGKGLEWVANIKQDGSEKYYVDSVKGRFTISRDNAKNSLYLQMNSLRVEDTAIYYCARDRPVAGASALWGQGTLVTVSS

SEQ ID NO: 66 - protein, 127 aa, synthetic construct
  REGION 1..127: note = Synthetic: 7H7 VL with signal sequence
MRVPAQLLGLLLLWLPGAKCDIQMTQSPSTLSASVGDRVTITCRASQSISGWLAWYQQKQGKAPKLLIYKASSLESGVPSRFSGSGSGTEFTLTISSLQPDDFATYYCQQYYGSSRTFGQGTNVEIK

SEQ ID NO: 67 - protein, 138 aa, synthetic construct
  REGION 1..138: note = Synthetic: 1B3 VH with signal sequence
MELGLSWVFLVAILEGVQCEVQLVESGGGLVQPGGSLRLSCAASGGTISTSWMSWVRQAPGKGLEWVANIKQDGSEKYYVDSVKGRFTISRDNAKNSLYLQMNSLRVEDTAMYYCARDRPVAGASALWGQGTLVTVSS

SEQ ID NO: 68 - protein, 127 aa, synthetic construct
  REGION 1..127: note = Synthetic: 1B3 VL with signal sequence
MRVPAQLLGLLLLWLPGAKCDIQMTQSPSTLSASVGDRVTITCRASQSISGWLAWYQQKPGKAPKLLIYKASSLESGVPSRFSGSGSGTEFTLTISSLQPDDFATYYCQQYYGSSRTFGQGTNVEIK

SEQ ID NO: 69 - protein, 138 aa, synthetic construct
  REGION 1..138: note = Synthetic: 3B6 VH with signal sequence
MELGLSWVFLVAILEGVQCEVQLVESGGGLVQPGGSLRLSCAASGGTTSTYWMSWVRQAPGKGLEWVANIKQDGSEKYYVDSVKGRFTISRDNAKNSLNLQMNSLRVEDTAIYYCARDRPVAGASALWGQGTLVTVSS

SEQ ID NO: 70 - protein, 127 aa, synthetic construct; identical to SEQ ID NO: 68
  REGION 1..127: note = Synthetic: 3B6 VL with signal sequence

SEQ ID NO: 71 - protein, 138 aa, synthetic construct
  REGION 1..138: note = Synthetic: 8B1 VH with signal sequence
MELGLSWVFLVAILEGVKCEVRLVESGGGLVQPGGSLRLSCAASGDIISTHWMSWVRQAPGKGLEWVANIKQDGSEKYYVDSVKGRFTISRDNAKNSLYLQMNTLRVEDTAIYYCTRDRPVAGASALWGQGTLVTVSS

SEQ ID NO: 72 - protein, 127 aa, synthetic construct
  REGION 1..127: note = Synthetic: 8B1 VL with signal sequence
MRVPAQLLGLLLLWLPGAKCDIQMTQSPSTLSASVGDRVTITCRASQSISGWLAWYQQKPGKAPKLLIYKASSLESGVPLRFSGSGSGTEFTLTISSLQPDDFATYYCQQYYGSSRTFGQGTNVEIK

SEQ ID NO: 73 - protein, 138 aa, synthetic construct
  REGION 1..138: note = Synthetic: 4A3 VH with signal sequence
MELGLSWVFLVAILEGVQCEVQLVESGGGLVQPGGSLRLSCAASGGIISSSWMSWVRQAPGKGLEWVANIKQDGSEKYYVDSVKGRFTISRDNAKDLLYLQMNSLRVEDTALYYCARDRPVAGASALWGQGTLVTVSS

SEQ ID NO: 74 - protein, 127 aa, synthetic construct; identical to SEQ ID NO: 68
  REGION 1..127: note = Synthetic: 4A3 VL with signal sequence

SEQ ID NO: 75 - protein, 138 aa, synthetic construct
  REGION 1..138: note = Synthetic: 9H9 VH with signal sequence
MELGLSWVFLVAILEGVQCEVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSS

SEQ ID NO: 76 - protein, 127 aa, synthetic construct; identical to SEQ ID NO: 68
  REGION 1..127: note = Synthetic: 9H9 VL with signal sequence

SEQ ID NO: 77 - protein, 119 aa, synthetic construct
  REGION 1..119: note = Synthetic: 7H7 VH without signal sequence
EVQLVESGGGLVQPGGSLRLSCAASGGTISTSWMSWVRQAPGKGLEWVANIKQDGSEKYYVDSVKGRFTISRDNAKNSLYLQMNSLRVEDTAIYYCARDRPVAGASALWGQGTLVTVSS

SEQ ID NO: 78 - protein, 107 aa, synthetic construct
  REGION 1..107: note = Synthetic: 7H7 VL without signal sequence
DIQMTQSPSTLSASVGDRVTITCRASQSISGWLAWYQQKQGKAPKLLIYKASSLESGVPSRFSGSGSGTEFTLTISSLQPDDFATYYCQQYYGSSRTFGQGTNVEIK

SEQ ID NO: 79 - protein, 119 aa, synthetic construct
  REGION 1..119: note = Synthetic: 1B3 VH without signal sequence
EVQLVESGGGLVQPGGSLRLSCAASGGTISTSWMSWVRQAPGKGLEWVANIKQDGSEKYYVDSVKGRFTISRDNAKNSLYLQMNSLRVEDTAMYYCARDRPVAGASALWGQGTLVTVSS

SEQ ID NO: 80 - protein, 107 aa, synthetic construct
  REGION 1..107: note = Synthetic: 1B3 VL without signal sequence
DIQMTQSPSTLSASVGDRVTITCRASQSISGWLAWYQQKPGKAPKLLIYKASSLESGVPSRFSGSGSGTEFTLTISSLQPDDFATYYCQQYYGSSRTFGQGTNVEIK

SEQ ID NO: 81 - protein, 119 aa, synthetic construct
  REGION 1..119: note = Synthetic: 3B6 VH without signal sequence
EVQLVESGGGLVQPGGSLRLSCAASGGTTSTYWMSWVRQAPGKGLEWVANIKQDGSEKYYVDSVKGRFTISRDNAKNSLNLQMNSLRVEDTAIYYCARDRPVAGASALWGQGTLVTVSS

SEQ ID NO: 82 - protein, 107 aa, synthetic construct; identical to SEQ ID NO: 80
  REGION 1..107: note = Synthetic: 3B6 VL without signal sequence

SEQ ID NO: 83 - protein, 119 aa, synthetic construct
  REGION 1..119: note = Synthetic: 8B1 VH without signal sequence
EVRLVESGGGLVQPGGSLRLSCAASGDIISTHWMSWVRQAPGKGLEWVANIKQDGSEKYYVDSVKGRFTISRDNAKNSLYLQMNTLRVEDTAIYYCTRDRPVAGASALWGQGTLVTVSS

SEQ ID NO: 84 - protein, 107 aa, synthetic construct
  REGION 1..107: note = Synthetic: 8B1 VL without signal sequence
DIQMTQSPSTLSASVGDRVTITCRASQSISGWLAWYQQKPGKAPKLLIYKASSLESGVPLRFSGSGSGTEFTLTISSLQPDDFATYYCQQYYGSSRTFGQGTNVEIK

SEQ ID NO: 85 - protein, 119 aa, synthetic construct
  REGION 1..119: note = Synthetic: 4A3 VH without signal sequence
EVQLVESGGGLVQPGGSLRLSCAASGGIISSSWMSWVRQAPGKGLEWVANIKQDGSEKYYVDSVKGRFTISRDNAKDLLYLQMNSLRVEDTALYYCARDRPVAGASALWGQGTLVTVSS

SEQ ID NO: 86 - protein, 107 aa, synthetic construct; identical to SEQ ID NO: 80
  REGION 1..107: note = Synthetic: 4A3 VL without signal sequence

SEQ ID NO: 87 - protein, 119 aa, synthetic construct
  REGION 1..119: note = Synthetic: 9H9 VH without signal sequence
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSS

SEQ ID NO: 88 - protein, 107 aa, synthetic construct; identical to SEQ ID NO: 80
  REGION 1..107: note = Synthetic: 9H9 VL without signal sequence

SEQ ID NO: 89 - DNA, 414 bp, synthetic construct
  misc_feature 1..414: note = Synthetic: 7H7 VH DNA Sequence with signal sequence
  variation 21: note = n is a, c, g, or t
  source 1..414: mol_type = other DNA
atggaattggggctgagctgngttttccttgttgctattttagaaggtgtccagtgtgaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatatattactgtgcgagagatcgtccagtggctggtgcgtcggccctctggggccagggaaccctggtcaccgtctcctca

SEQ ID NO: 90 - DNA, 381 bp, synthetic construct
  misc_feature 1..381: note = Synthetic: 7H7 VL DNA Sequence with signal sequence
  source 1..381: mol_type = other DNA
atgagggtccccgctcagctcctggggctcctgctgctctggctcccaggtgccaaatgtgacatccagatgacccagtctccttccaccctgtctgcatctgtaggagacagagtcaccatcacttgccgggccagtcagagtataagtggctggttggcctggtatcagcagaaacaagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 91 - DNA, 414 bp, synthetic construct
  misc_feature 1..414: note = Synthetic: 1B3 VH DNA Sequence with signal sequence
  source 1..414: mol_type = other DNA
atggaattggggctgagctgggttttccttgttgctattttagaaggtgtccagtgtgaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaagacacggctatgtattactgtgcgagagatcgtccagtggctggtgcgtcggccctctggggccagggaaccctggtcaccgtctcctca

SEQ ID NO: 92 - DNA, 381 bp, synthetic construct
  misc_feature 1..381: note = Synthetic: 1B3 VL DNA Sequence with signal sequence
  source 1..381: mol_type = other DNA
atgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 93 - DNA, 414 bp, synthetic construct
  misc_feature 1..414: note = Synthetic: 3B6 VH DNA Sequence with signal sequence
  source 1..414: mol_type = other DNA
atggaattggggctgagctgggttttccttgttgctattttagaaggtgtccagtgtgaggtgcaactggtggagtctgggggaggcttggtccagcctggggggtccctgagactctcctgtgcagcctctggaggcacaaccagtacctattggatgagctgggtccgccaggctccagggaaggggctggaatgggtggccaacataaagcaagatggaagtgagaaatattatgtggactctgtgaagggccgattcaccatctccagagacaacgccaagaactcactgaatttgcaaatgaacagcctgagagtcgaggacacggctatatattactgtgcgagagatcgtccagtggctggtgcgtcggccctctggggccagggaaccctggtcaccgtctcctca

SEQ ID NO: 94 - DNA, 381 bp, synthetic construct; identical to SEQ ID NO: 92
  misc_feature 1..381: note = Synthetic: 3B6 VL DNA Sequence with signal sequence
  source 1..381: mol_type = other DNA

SEQ ID NO: 95 - DNA, 414 bp, synthetic construct
  misc_feature 1..414: note = Synthetic: 8B1 VH DNA Sequence with signal sequence
  source 1..414: mol_type = other DNA
atggaattggggctgagctgggttttccttgttgctattttagaaggtgtcaagtgtgaggtgcgactggtggagtctgggggaggcttggtccagcctggggggtccctgagactctcctgtgcagcctctggagacataattagtacccattggatgagctgggtccgccaggctccagggaaggggctggaatgggtggccaacataaaacaagatggaagtgagaagtattatgtggactctgtgaagggccgattcaccatctccagagacaacgccaagaactcactgtatttgcaaatgaacaccctgagagtcgaggacacggctatatattactgtacgagagatcgtccagtggctggtgcgtcggccctctggggccagggaaccctggtcaccgtctcctca

SEQ ID NO: 96 - DNA, 381 bp, synthetic construct
  misc_feature 1..381: note = Synthetic: 8B1 VL DNA Sequence with signal sequence
  source 1..381: mol_type = other DNA
atgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccattaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 97 - DNA, 414 bp, synthetic construct
  misc_feature 1..414: note = Synthetic: 4A3 VH DNA Sequence with signal sequence
  source 1..414: mol_type = other DNA
atggaattggggctgagctgggttttccttgttgctattttagaaggtgtccagtgtgaggtgcaactggtggagtctgggggaggcttggtccagccgggggggtccctgagactctcctgtgcagcctctggaggcatcattagttcctcttggatgagctgggtccgccaggctccagggaaggggctggaatgggtggccaacataaagcaagatggaagtgagaaatattatgtggactctgtgaagggccgattcaccatctccagagacaacgccaaagacttactgtatttgcaaatgaacagcctgagagtcgaggacacggctttatattactgtgcgagagatcgtccagtggctggtgcgtcggccctctggggccagggaaccctggtcaccgtctcctct

SEQ ID NO: 98 - DNA, 381 bp, synthetic construct; identical to SEQ ID NO: 92
  misc_feature 1..381: note = Synthetic: 4A3 VL DNA Sequence with signal sequence
  source 1..381: mol_type = other DNA

SEQ ID NO: 99 - DNA, 414 bp, synthetic construct
  misc_feature 1..414: note = Synthetic: 9H9 VH DNA Sequence with signal sequence
  source 1..414: mol_type = other DNA
atggaattggggctgagctgggttttccttgttgctattttagaaggtgtccagtgtgaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctca

SEQ ID NO: 100 - DNA, 381 bp, synthetic construct; identical to SEQ ID NO: 92
  misc_feature 1..381: note = Synthetic: 9H9 VL DNA Sequence with signal sequence
  source 1..381: mol_type = other DNA

SEQ ID NO: 101 - DNA, 357 bp, synthetic construct
  misc_feature 1..357: note = Synthetic: 7H7 VH DNA Sequence without signal sequence
  source 1..357: mol_type = other DNA
gaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatatattactgtgcgagagatcgtccagtggctggtgcgtcggccctctggggccagggaaccctggtcaccgtctcctca

SEQ ID NO: 102 - DNA, 321 bp, synthetic construct
  misc_feature 1..321: note = Synthetic: 7H7 VL DNA Sequence without signal sequence
  source 1..321: mol_type = other DNA
gacatccagatgacccagtctccttccaccctgtctgcatctgtaggagacagagtcaccatcacttgccgggccagtcagagtataagtggctggttggcctggtatcagcagaaacaagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 103 - DNA, 357 bp, synthetic construct
  misc_feature 1..357: note = Synthetic: 1B3 VH DNA Sequence without signal sequence
  source 1..357: mol_type = other DNA
gaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaagacacggctatgtattactgtgcgagagatcgtccagtggctggtgcgtcggccctctggggccagggaaccctggtcaccgtctcctca

SEQ ID NO: 104 - DNA, 321 bp, synthetic construct
  misc_feature 1..321: note = Synthetic: 1B3 VL DNA Sequence without signal sequence
  source 1..321: mol_type = other DNA
gacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 105 - DNA, 357 bp, synthetic construct
  misc_feature 1..357: note = Synthetic: 3B6 VH DNA Sequence without signal sequence
  source 1..357: mol_type = other DNA
gaggtgcaactggtggagtctgggggaggcttggtccagcctggggggtccctgagactctcctgtgcagcctctggaggcacaaccagtacctattggatgagctgggtccgccaggctccagggaaggggctggaatgggtggccaacataaagcaagatggaagtgagaaatattatgtggactctgtgaagggccgattcaccatctccagagacaacgccaagaactcactgaatttgcaaatgaacagcctgagagtcgaggacacggctatatattactgtgcgagagatcgtccagtggctggtgcgtcggccctctggggccagggaaccctggtcaccgtctcctca

SEQ ID NO: 106 - DNA, 321 bp, synthetic construct; identical to SEQ ID NO: 104
  misc_feature 1..321: note = Synthetic: 3B6 VL DNA Sequence without signal sequence
  source 1..321: mol_type = other DNA

SEQ ID NO: 107 - DNA, 357 bp, synthetic construct
  misc_feature 1..357: note = Synthetic: 8B1 VH DNA Sequence without signal sequence
  source 1..357: mol_type = other DNA
gaggtgcgactggtggagtctgggggaggcttggtccagcctggggggtccctgagactctcctgtgcagcctctggagacataattagtacccattggatgagctgggtccgccaggctccagggaaggggctggaatgggtggccaacataaaacaagatggaagtgagaagtattatgtggactctgtgaagggccgattcaccatctccagagacaacgccaagaactcactgtatttgcaaatgaacaccctgagagtcgaggacacggctatatattactgtacgagagatcgtccagtggctggtgcgtcggccctctggggccagggaaccctggtcaccgtctcctca

SEQ ID NO: 108 - DNA, 321 bp, synthetic construct
  misc_feature 1..321: note = Synthetic: 8B1 VL DNA Sequence without signal sequence
  source 1..321: mol_type = other DNA
gacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccattaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 109 - DNA, 357 bp, synthetic construct
  misc_feature 1..357: note = Synthetic: 4A3 VH DNA Sequence without signal sequence
  source 1..357: mol_type = other DNA
gaggtgcaactggtggagtctgggggaggcttggtccagccgggggggtccctgagactctcctgtgcagcctctggaggcatcattagttcctcttggatgagctgggtccgccaggctccagggaaggggctggaatgggtggccaacataaagcaagatggaagtgagaaatattatgtggactctgtgaagggccgattcaccatctccagagacaacgccaaagacttactgtatttgcaaatgaacagcctgagagtcgaggacacggctttatattactgtgcgagagatcgtccagtggctggtgcgtcggccctctggggccagggaaccctggtcaccgtctcctct

SEQ ID NO: 110 - DNA, 321 bp, synthetic construct; identical to SEQ ID NO: 104
  misc_feature 1..321: note = Synthetic: 4A3 VL DNA Sequence without signal sequence
  source 1..321: mol_type = other DNA

SEQ ID NO: 111 - DNA, 357 bp, synthetic construct
  misc_feature 1..357: note = Synthetic: 9H9 VH DNA Sequence without signal sequence
  source 1..357: mol_type = other DNA
gaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctca

SEQ ID NO: 112 - DNA, 321 bp, synthetic construct; identical to SEQ ID NO: 104
  misc_feature 1..321: note = Synthetic: 9H9 VL DNA Sequence without signal sequence
  source 1..321: mol_type = other DNA

SEQ ID NO: 113 - protein, 7 aa, synthetic construct
  REGION 1..7: note = Synthetic: 3C2 VH CDR1 (Chothia)
TGSISGY

SEQ ID NO: 114 - protein, 5 aa, synthetic construct
  REGION 1..5: note = Synthetic: 3C2 VH CDR2 (Chothia)
YYSGS

SEQ ID NO: 115 - protein, 10 aa, synthetic construct
  REGION 1..10: note = Synthetic: 3C2 VH CDR3 (Chothia)
YPLIRGAFDY

SEQ ID NO: 116 - protein, 16 aa, synthetic construct
  REGION 1..16: note = Synthetic: 3C2 VL CDR1 (Chothia)
RSSQNLLHTNGYNYLD

SEQ ID NO: 117 - protein, 7 aa, synthetic construct
  REGION 1..7: note = Synthetic: 3C2 VL CDR2 (Chothia)
LGSNRAS

SEQ ID NO: 118 - protein, 9 aa, synthetic construct
  REGION 1..9: note = Synthetic: 3C2 VL CDR3 (Chothia)
MQALQTPLT

SEQ ID NO: 119 - protein, 7 aa, synthetic construct
  REGION 1..7: note = Synthetic: 2B3 VH CDR1 (Chothia)
GYTFTGY

SEQ ID NO: 120 - protein, 6 aa, synthetic construct
  REGION 1..6: note = Synthetic: 2B3 VH CDR2 (Chothia)
NPNSGG

SEQ ID NO: 121 - protein, 18 aa, synthetic construct
  REGION 1..18: note = Synthetic: 2B3 VH CDR3 (Chothia)
DRLVLPWFGEIFPDAFDI

SEQ ID NO: 122 - protein, 11 aa, synthetic construct
  REGION 1..11: note = Synthetic: 2B3 VL CDR1 (Chothia)
RASQSIRSNLA

SEQ ID NO: 123 - protein, 7 aa, synthetic construct
  REGION 1..7: note = Synthetic: 2B3 VL CDR2 (Chothia)
GASTRAT

SEQ ID NO: 124 - protein, 9 aa, synthetic construct
  REGION 1..9: note = Synthetic: 2B3 VL CDR3 (Chothia)
QQYNNWPLT

SEQ ID NO: 125 - protein, 7 aa, synthetic construct
  REGION 1..7: note = Synthetic: 7H7 VH CDR1 (Chothia)
GGTISTS

SEQ ID NO: 126 - protein, 6 aa, synthetic construct
  REGION 1..6: note = Synthetic: 7H7 VH CDR2 (Chothia)
KQDGSE

SEQ ID NO: 127 - protein, 10 aa, synthetic construct
  REGION 1..10: note = Synthetic: 7H7 VH CDR3 (Chothia)
DRPVAGASAL

SEQ ID NO: 128 - protein, 11 aa, synthetic construct
  REGION 1..11: note = Synthetic: 7H7 VL CDR1 (Chothia)
RASQSISGWLA

SEQ ID NO: 129 - protein, 7 aa, synthetic construct
  REGION 1..7: note = Synthetic: 7H7 VL CDR2 (Chothia)
KASSLES

SEQ ID NO: 130 - protein, 9 aa, synthetic construct
  REGION 1..9: note = Synthetic: 7H7 VL CDR3 (Chothia)
QQYYGSSRT

SEQ ID NO: 131 - protein, 6 aa, synthetic construct
  REGION 1..6: note = Synthetic: 1B3 VH CDR1 (Chothia)
GGTSTS

SEQ ID NO: 132 - protein, 6 aa, synthetic construct
  REGION 1..6: note = Synthetic: 1B3 VH CDR2 (Chothia)
KQDGSE

SEQ ID NO: 133 - protein, 10 aa, synthetic construct
  REGION 1..10: note = Synthetic: 1B3 VH CDR3 (Chothia)
DRPVAGASAL

SEQ ID NO: 134 - protein, 11 aa, synthetic construct
  REGION 1..11: note = Synthetic: 1B3 VL CDR1 (Chothia)
RASQSISGWLA

SEQ ID NO: 135 - protein, 7 aa, synthetic construct
  REGION 1..7: note = Synthetic: 1B3 VL CDR2 (Chothia)
KASSLES

SEQ ID NO: 136 - protein, 9 aa, synthetic construct
  REGION 1..9: note = Synthetic: 1B3 VL CDR3 (Chothia)
QQYYGSSRT

SEQ ID NO: 137 - protein, 7 aa, synthetic construct
  REGION 1..7: note = Synthetic: 3B6 VH CDR1 (Chothia)
GGTTSTY

SEQ ID NO: 138 - protein, 6 aa, synthetic construct
  REGION 1..6: note = Synthetic: 3B6 VH CDR2 (Chothia)
KQDGSE

SEQ ID NO: 139 - protein, 10 aa, synthetic construct
  REGION 1..10: note = Synthetic: 3B6 VH CDR3 (Chothia)
DRPVAGASAL

SEQ ID NO: 140 - protein, 11 aa, synthetic construct
  REGION 1..11: note = Synthetic: 3B6 VL CDR1 (Chothia)
RASQSISGWLA

SEQ ID NO: 141 - protein, 7 aa, synthetic construct
  REGION 1..7: note = Synthetic: 3B6 VL CDR2 (Chothia)
KASSLES

SEQ ID NO: 142 - protein, 9 aa, synthetic construct
  REGION 1..9: note = Synthetic: 3B6 VL CDR3 (Chothia)
QQYYGSSRT

SEQ ID NO: 143 - protein, 7 aa, synthetic construct
  REGION 1..7: note = Synthetic: 8B1 VH CDR1 (Chothia)
GDIISTH

SEQ ID NO: 144 - protein, 6 aa, synthetic construct
  REGION 1..6: note = Synthetic: 8B1 VH CDR2 (Chothia)
KQDGSE

SEQ ID NO: 145 - protein, 10 aa, synthetic construct
  REGION 1..10: note = Synthetic: 8B1 VH CDR3 (Chothia)
DRPVAGASAL

SEQ ID NO: 146 - protein, 11 aa, synthetic construct
  REGION 1..11: note = Synthetic: 8B1 VL CDR1 (Chothia)
RASQSISGWLA

SEQ ID NO: 147 - protein, 7 aa, synthetic construct
  REGION 1..7: note = Synthetic: 8B1 VL CDR2 (Chothia)
KASSLES

SEQ ID NO: 148 - protein, 9 aa, synthetic construct
  REGION 1..9: note = Synthetic: 8B1 VL CDR3 (Chothia)
QQYYGSSRT

SEQ ID NO: 149 - protein, 7 aa, synthetic construct
  REGION 1..7: note = Synthetic: 4A3 VH CDR1 (Chothia)
GGIISSS

SEQ ID NO: 150 - protein, 6 aa, synthetic construct
  REGION 1..6: note = Synthetic: 4A3 VH CDR2 (Chothia)
KQDGSE

SEQ ID NO: 151 - protein, 10 aa, synthetic construct
  REGION 1..10: note = Synthetic: 4A3 VH CDR3 (Chothia)
DRPVAGASAL

SEQ ID NO: 152 - protein, 11 aa, synthetic construct
  REGION 1..11: note = Synthetic: 4A3 VL CDR1 (Chothia)
RASQSISGWLA

SEQ ID NO: 153 - protein, 7 aa, synthetic construct
  REGION 1..7: note = Synthetic: 4A3 VL CDR2 (Chothia)
KASSLES

SEQ ID NO: 154 - protein, 9 aa, synthetic construct
  REGION 1..9: note = Synthetic: 4A3 VL CDR3 (Chothia)
QQYYGSSRT

SEQ ID NO: 155 - protein, 7 aa, synthetic construct
  REGION 1..7: note = Synthetic: 9H9 VH CDR1 (Chothia)
GGIISTY

SEQ ID NO: 156 - protein, 6 aa, synthetic construct
  REGION 1..6: note = Synthetic: 9H9 VH CDR2 (Chothia)
KQDGSE

SEQ ID NO: 157 - protein, 10 aa, synthetic construct
  REGION 1..10: note = Synthetic: 9H9 VH CDR3 (Chothia)
DRPVAGASAL

SEQ ID NO: 158 - protein, 11 aa, synthetic construct
  REGION 1..11: note = Synthetic: 9H9 VL CDR1 (Chothia)
RASQSISGWLA

SEQ ID NO: 159 - protein, 7 aa, synthetic construct
  REGION 1..7: note = Synthetic: 9H9 VL CDR2 (Chothia)
KASSLES

SEQ ID NO: 160 - protein, 9 aa, synthetic construct
  REGION 1..9: note = Synthetic: 9H9 VL CDR3 (Chothia)
QQYYGSSRT

SEQ ID NO: 161 - skipped

SEQ ID NO: 162 - protein, 17 aa, synthetic construct
  REGION 1..17: note = Synthetic: CD27 antibody VH CDR2 consensus sequence (Kabat)
  VARIANT 1: note = X is Absent or W
  VARIANT 2: note = X is Y or I
  VARIANT 4: note = X is Y or P
  VARIANT 5: note = X is Y or N
  VARIANT 8: note = X is S or G
  VARIANT 11: note = X is Y or S
  VARIANT 12: note = X is N or A
  VARIANT 13: note = X is P or Q
  VARIANT 14: note = X is S or K
  VARIANT 15: note = X is L or F
  VARIANT 16: note = X is K or Q
  VARIANT 17: note = X is S or D
XXNXXSGXTNXXXXXXX

SEQ ID NO: 163 - protein, 21 aa, synthetic construct
  REGION 1..21: note = Synthetic: CD27 antibody VH CDR3 consensus sequence (Kabat)
  VARIANT 1: note = X is Absent or D
  VARIANT 2: note = X is Absent or R
  VARIANT 3: note = X is Absent or L
  VARIANT 4: note = X is Absent or V
  VARIANT 5: note = X is Absent or L
  VARIANT 6: note = X is Absent or P
  VARIANT 7: note = X is Absent or W
  VARIANT 8: note = X is Absent or F
  VARIANT 9: note = X is Absent or G
  VARIANT 10: note = X is Absent or E
  VARIANT 11: note = X is Absent or I
  VARIANT 12: note = X is AbsentY or F
  VARIANT 14: note = X is L or Absent
  VARIANT 15: note = X is I or Absent
  VARIANT 16: note = X is R or Absent
  VARIANT 17: note = X is G or D
  VARIANT 21: note = X is Y or I S or A
XXXXXXXXXXXXPXXXXAFDX

SEQ ID NO: 164 - protein, 20 aa, synthetic construct
  REGION 1..20: note = Synthetic: CD27 antibody VL CDR1 consensus sequence (Kabat)
  VARIANT 2: note = X is S or A
  VARIANT 5: note = X is Absent or S
  VARIANT 6: note = X is Absent or I
  VARIANT 7: note = X is Absent or R
  VARIANT 8: note = X is Absent or S
  VARIANT 11: note = X is L or A
  VARIANT 12: note = X is H or Absent
  VARIANT 13: note = X is T or Absent
  VARIANT 14: note = X is N or Absent
  VARIANT 15: note = X is G or Absent
  VARIANT 16: note = X is Y or Absent
  VARIANT 17: note = X is N or Absent
  VARIANT 18: note = X is Y or Absent
  VARIANT 19: note = X is L or Absent
  VARIANT 20: note = X is D or Absent
RXSQXXXXNLXXXXXXXXXX

SEQ ID NO: 165 - skipped

SEQ ID NO: 166 - protein, 9 aa, synthetic construct
  REGION 1..9: note = Synthetic: CD27 antibody VL CDR3 consensus sequence (Kabat)
  VARIANT 1: note = X is M or Q
  VARIANT 3: note = X is A or Y
  VARIANT 4: note = X is L or N
  VARIANT 5: note = X is Q or N
  VARIANT 6: note = X is T or W
XQXXXXPLT

SEQ ID NO: 167 - skipped

SEQ ID NO: 168 - protein, 17 aa, synthetic construct
  REGION 1..17: note = Synthetic: PD-L1 antibody VH CDR2 consensus sequence (Kabat)
NIKQDGSEKYYVDSVKG

SEQ ID NO: 169 - protein, 10 aa, synthetic construct
  REGION 1..10: note = Synthetic: PD-L1 antibody VH CDR3 consensus sequence (Kabat)
DRPVAGASAL

SEQ ID NO: 170 - protein, 11 aa, synthetic construct
  REGION 1..11: note = Synthetic: PD-L1 antibody VL CDR1 consensus sequence (Kabat)
RASQSISGWLA

SEQ ID NO: 171 - protein, 7 aa, synthetic construct
  REGION 1..7: note = Synthetic: PD-L1 antibody VL CDR2 consensus sequence (Kabat)
KASSLES

SEQ ID NO: 172 - protein, 9 aa, synthetic construct
  REGION 1..9: note = Synthetic: PD-L1 antibody VL CDR3 consensus sequence (Kabat)
QQYYGSSRT

SEQ ID NO: 173 - protein, 260 aa, Homo sapiens
  REGION 1..260: note = misc_feature - CD27 amino acid sequence
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 174 - protein, 193 aa, Homo sapiens
  REGION 1..193: note = misc_feature - CD70 amino acid sequence
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 175 - protein, 288 aa, Homo sapiens
  REGION 1..288: note = misc_feature - PD1 amino acid sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 176 - protein, 176 aa, Homo sapiens
  REGION 1..176: note = misc_feature - PD-L1 amino acid sequence (isoform b precursor)
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 177 - protein, 127 aa, synthetic construct
  REGION 1..127: note = Synthetic: 2B3 VH (modified)
QVQLVQSGAEVKKPGASVKVSCKASGYTFTGYYIHWVRQAPGQGLEWMGWINPNSGGTNSAQKFQDRVTITRDTSINTAYMELSRLRSDDTAVYFCARDRLVLPWFGEIFPDAFDIWGQGTLVTVSS

SEQ ID NO: 178 - protein, 107 aa, synthetic construct
  REGION 1..107: note = Synthetic: 2B3 VL (modified)
EIVMTQSPATLSVSPGERATLSCRASQSIRSNLAWYQQKPGQAPRLLIYGASTRATGIPARFSGSGSGTEFTLTISSLQSEDFAVYYCQQYNNWPLTFGGGTKVEIK

SEQ ID NO: 179 - protein, 711 aa, synthetic construct
  REGION 1..711: note = Synthetic: Full 9H9-2B3 (CDX-527) heavy chain sequence
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSEIVMTQSPATLSVSPGERATLSCRASQSIRSNLAWYQQKPGQAPRLLIYGASTRATGIPARFSGSGSGTEFTLTISSLQSENFAVYYCQQYNNWPLTFGCGTKVEIKGGGGSGGGGSGGGGSGGGGSQVQLVQSGAEVKKPGASVKVSCKASGYTFTGYYIHWVRQAPGQCLEWMGWINPNSGGTNSAQKFQDRVTITRVTSINTAYMELSRLRSDDTAVYFCARDRLVLPWFGEIFPDAFDIWGQGTLVTVSS

SEQ ID NO: 180 - protein, 214 aa, synthetic construct
  REGION 1..214: note = Synthetic: 9H9-2B3 (CDX-527) light chain sequence
DIQMTQSPSTLSASVGDRVTITCRASQSISGWLAWYQQKPGKAPKLLIYKASSLESGVPSRFSGSGSGTEFTLTISSLQPDDFATYYCQQYYGSSRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 711 aa, synthetic construct
  REGION 1..711: note = Synthetic: Full 9H9-2B3(DD) heavy chain sequence
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSEIVMTQSPATLSVSPGERATLSCRASQSIRSNLAWYQQKPGQAPRLLIYGASTRATGIPARFSGSGSGTEFTLTISSLQSEDFAVYYCQQYNNWPLTFGCGTKVEIKGGGGSGGGGSGGGGSGGGGSQVQLVQSGAEVKKPGASVKVSCKASGYTFTGYYIHWVRQAPGQCLEWMGWINPNSGGTNSAQKFQDRVTITRDTSINTAYMELSRLRSDDTAVYFCARDRLVLPWFGEIFPDAFDIWGQGTLVTVSS

SEQ ID NO: 182 - protein, 214 aa, synthetic construct; identical to SEQ ID NO: 180
  REGION 1..214: note = Synthetic: 9H9-2B3(DD) light chain sequence

SEQ ID NO: 183 - protein, 168 aa, synthetic construct
  REGION 1..168: note = Synthetic: ECD of huCD27
TPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSD

SEQ ID NO: 184 - protein, 168 aa, synthetic construct
  REGION 1..168: note = Synthetic: ECD of huCD27 mutated
TPAPKSCPERHYWAQGKLCCQMCEPGTFLVKDCDQHRKAAQCDPCIPGVSFSPDHHTRPHCESCRHCNSGLLVRNCTITANAECSCAAAWQCRDKECTECDPLPNPSLTARSSQALSPHPQPTHLPYVSEMLEARTAGHMQTLADFRQLPARTLSTHWPPQRSLCSSD

SEQ ID NO: 185 - DNA, 357 bp, synthetic construct
  misc_feature 1..357: note = Synthetic: NA sequence of 9H9x2B3(DD) variable domain
  source 1..357: mol_type = other DNA
gaagtgcaactggtggagtcgggtggtggactcgtgcagcccggcggatccctgagactctcttgtgccgcatcgggcggcattattagcacttactggatgtcatgggtcagacaggcaccgggaaagggcttggaatgggtggcgaatatcaagcaggatggatccgagaagtactacgtggactccgtgaagggcagattcaccatttcccgggacaacgccaagaactcgctctatctgcaaatgaactcgttgcgggtggaagatactgccatgtactactgcgcccgggaccggcctgtggccggggcgtcggccctctggggccagggcactctggtcaccgtgtcctct

SEQ ID NO: 186 - DNA, 786 bp, synthetic construct
  misc_feature 1..786: note = Synthetic: NA sequence of 9H9x2B3(DD) scFv domain
  source 1..786: mol_type = other DNA
ggctccagcgggggtggcggttccgagatcgtgatgactcagagcccggcaaccctgtccgtgtctccgggggagcgggctactctttcctgccgggcatcccagtccatccggtcgaaccttgcgtggtaccaacagaagcctggacaggcgccccgcctgctgatctacggggcgtcgactagggccaccggcatcccggcccgcttctccgggtccggatccggcaccgaattcaccctcaccatctcgagcctgcagtccgaaaacttcgccgtctactactgccagcagtacaacaactggccgctgacattcggatgcggaaccaaagtggaaatcaagggcggcggcggatccggcggtggcggcagcggcggtggaggatccggtggcggcggttcacaagtgcagctggtgcagtcaggcgccgaagtcaagaagcccggggccagcgtgaaagtcagctgcaaggcttccggatacaccttcacgggttactacattcactgggttcgccaagcgcccgggcagtgtctggagtggatgggatggatcaaccctaactcggggggaaccaactcggcccaaaagttccaggaccgggtcaccattacaagagtcacgtccatcaacactgcctacatggagttgagccggctgcgatcagacgacaccgccgtgtacttctgcgcgagggaccgcctcgtcctcccgtggtttggagagatcttcccggatgccttcgacatttggggacaggggaccctcgtgactgtgtccagc

SEQ ID NO: 187 - DNA, 321 bp, synthetic construct
  misc_feature 1..321: note = Synthetic: NA sequence of the 9H9x2B3(DD) light chain variable domain
  source 1..321: mol_type = other DNA
gatatccagatgacccagagcccgtccaccctttccgcgagcgtcggcgacagagtgaccattacttgtcgggcctcgcaaagcatctccggctggctggcttggtaccagcaaaagcctggaaaggcccctaagctgctgatctacaaggcctcatccctggagtccggagtgccttcacgcttttcggggagcggatcggggactgagttcaccctcaccatttcctccctgcaacccgacgatttcgcgacatactactgccagcagtactacggttcctcgcgcacgttcggacagggcactaacgtcgagatcaag

(19) United States
(12) Patent Application Publication
(10) Pub. No.: US 2025/0346671 A1
(43) Pub. Date: Nov. 13, 2025

(54) ANTI-PD-L1 ANTIBODIES AND METHODS OF USE THEREOF

(71) Applicant: Celldex Therapeutics, Inc., Hampton, NJ (US)

(72) Inventors: Tibor Keler, Pipersville, PA (US); Joel Goldstein, Hopewell, NJ (US); Laura A. Vitale, Doylestown, PA (US); Lizhen He, Zhenghe County (CN)

(21) Appl. No.: 18/962,161

(22) Filed: Nov. 27, 2024

Related U.S. Application Data
(63) Continuation of application No. 17/047,656, filed on Oct. 14, 2020, now abandoned.
(60) Provisional application No. 62/658,899, filed on Apr. 17, 2018.
(60) Provisional application No. 62/826,091, filed on Mar. 29, 2019.

(51) Int. Cl. C07K 16/28; A61K 39/00; A61P 35/00

(52) U.S. Cl. CPC C07K 16/2827; A61P 35/00; C07K 16/2878; A61K 39/0011; A61K 2039/505; A61K 2039/507; A61K 2039/545; C07K 2317/21; C07K 2317/31; C07K 2317/33; C07K 2317/34; C07K 2317/35; C07K 2317/565; C07K 2317/622; C07K 2317/732; C07K 2317/734; C07K 2317/74; C07K 2317/76; C07K 2317/90; C07K 2317/92

(57) ABSTRACT

Provided herein are novel anti-CD27 and anti-PD-L1 antibodies, and binding domains thereof, as well as bispecific constructs and anti-CD27 binding domain linked to an anti-PD-L1 binding domain. Also provided herein are methods of stimulating T cell activity, methods of inducing or enhancing an immune response, and methods of treating a disease or condition (e.g., cancer) by administering the bispecific constructs, antibodies, or antigen binding fragments thereof, or compositions described herein to a patient in need thereof.

### RELATED APPLICATIONS

This application is a continuation of U.S. patent application Ser. No. 17/047,656 filed on Oct. 14, 2020, which claims priority to 35 U.S.C. 371 International Application No. PCT/US2019/027897, filed on Apr. 17, 2019, which claims the benefit of priority of U.S. Provisional Application No. 62/658,899 (filed on Apr. 17, 2018) and U.S. Provisional Application No. 62/826,091 (filed on Mar. 29, 2019). The contents of the aforementioned applications are hereby incorporated by reference in their entirety.

### REFERENCE TO SEQUENCE LISTING

The instant application contains a Sequence Listing which has been submitted electronically in xml format and is hereby incorporated by reference in its entirety. Said xml copy, created on Jan. 15, 2025, is named CDJ_400USCN_Sequence_Listing.xml and is 273,034 bytes in size.

### I. BACKGROUND OF THE INVENTION

Interactions between T cells and antigen-presenting cells involve a variety of accessory molecules that facilitate in the generation of an immune response. One such molecule is CD27, which binds CD70 and belongs to the tumor necrosis factor receptor (TNF-R) superfamily (Ranheim, E. A. et al. (1995) Blood, 85 (12): 3556-65). CD27 typically exists as a glycosylated, type I transmembrane protein, frequently in the form of homodimers with a disulfide bridge linking the two monomers. The disulfide bridge is in the extracellular domain close to the membrane (Camerini et al. (1991) J. Immunol., 147:3165-69). CD27 may also be expressed in a soluble form (see, e.g., van Oers, M. H. et al. (1993) Blood 82 (11): 3430-6 and Loenen, W. A. et al. (1992) Eur. J. Immunol., 22:447). Cross-linking the CD27 antigen on T cells provides a costimulatory signal that, in concert with T-cell receptor crosslinking, can induce T-cell proliferation and cellular immune activation.

CD27 is expressed on mature thymocytes, on most CD4+ and CD8+ peripheral blood T cells, natural killer cells and B cells (Kobata, T. et al. (1995) Proc. Natl. Acad. Sci. USA, 92 (24): 11249-53). CD27 is also highly expressed on B cell non-Hodgkin's lymphomas and B cell chronic lymphocytic leukemias (Ranheim, E. A. et al. (1995) Blood, 85 (12): 3556-65). Additionally, increased levels of soluble CD27 protein have been identified in sera or sites of disease activity in parasitic infection, cytomegalovirus (CMV) infection, sarcoidosis, multiple sclerosis, and B-cell chronic lymphocytic leukemia (Loenen, W. A. et al. (1992) Eur. J. Immunol, 22:447).

Programmed death-ligand 1 (PD-L1) is a 40 kDa type 1 transmembrane protein that has been speculated to play a major role in suppressing the immune system during particular events such as pregnancy, tissue allografts, autoimmune disease, and other disease states such as hepatitis. Normally the immune system reacts to foreign antigens that are associated with exogenous or endogenous danger signals, which triggers a proliferation of antigen-specific CD8+ T cells and/or CD4+ helper cells. The binding of PD-L1 to PD-1 transmits an inhibitory signal that reduces the proliferation of these T cells and can also induce apoptosis, which is further mediated by a lower regulation of the gene Bcl-2. PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Despite advances in multimodal therapy, there is a need in the art for new and improved therapeutic agents to treat conditions or diseases (e.g., in which stimulation of an immune response is desired). Accordingly, it is an object of the present invention to provide improved methods for treating subjects with such conditions or diseases (e.g., cancer).

### II. Summary of the Invention

Provided herein are novel anti-CD27 and anti-PD-L1 antibodies, and binding domains thereof, as well as bispecific constructs and multispecific constructs comprising an anti-CD27 binding domain linked to an anti-PD-L1 binding domain. Also provided herein are methods of stimulating T cell activity, methods of inducing or enhancing an immune response, and methods of treating a disease or condition (e.g., cancer) by administering the bispecific or multispecific constructs, antibodies, or antigen binding fragments thereof, or compositions described herein to a patient in need thereof.

An exemplary anti-CD27 antibody is antibody 3C2 as described herein. In one embodiment, the anti-CD27 antibody or binding domain thereof comprises the heavy and light chain CDRs or variable regions of antibody 3C2. In another embodiment, the antibody or binding domain thereof comprises the CDR1, CDR2, and CDR3 domains of the heavy chain variable region of antibody 3C2 having the sequence set forth in SEQ ID NO: 17, and the CDR1, CDR2 and CDR3 domains of the light chain variable region of antibody 3C2 having the sequence set forth in SEQ ID NO:18. In another embodiment, the antibody or binding domain thereof comprises heavy chain CDR1, CDR2 and CDR3 domains having the sequences set forth in SEQ ID NOs: 1, 2, and 3, respectively, or conservative sequence modifications thereof, and light chain CDR1, CDR2 and CDR3 domains having the sequences set forth in SEQ ID NOs: 4, 5, and 6, respectively, or conservative sequence modifications thereof. In another embodiment, the antibody or binding domain thereof comprises a heavy chain variable region having the amino acid sequence set forth in SEQ ID NO: 17. In another embodiment, the antibody or binding domain thereof comprises a light chain variable region having the amino acid sequence set forth in SEQ ID NO:18. In another embodiment, the antibody or binding domain thereof comprises heavy and light chain variable regions having the amino acid sequences set forth in SEQ ID NO: 17 and SEQ ID NO: 18, respectively.

Another exemplary anti-CD27 antibody is antibody 2B3 as described herein. In one embodiment, the anti-CD27 antibody or binding domain thereof comprises the heavy and light chain CDRs or variable regions of antibody 2B3. In another embodiment, the antibody or binding domain thereof comprises the CDR1, CDR2, and CDR3 domains of the heavy chain variable region of antibody 2B3 having the sequence set forth in SEQ ID NO: 19, and the CDR1, CDR2 and CDR3 domains of the light chain variable region of antibody 3C2 having the sequence set forth in SEQ ID NO:20. In another embodiment, the antibody or binding domain thereof comprises heavy chain CDR1, CDR2 and CDR3 domains having the sequences set forth in SEQ ID NOs: 7, 8, and 9, respectively, or conservative sequence modifications thereof, and light chain CDR1, CDR2 and CDR3 domains having the sequences set forth in SEQ ID NOs: 10, 11, and 12, respectively, or conservative sequence modifications thereof. In another embodiment, the antibody or binding domain thereof comprises a heavy chain variable region having the amino acid sequences set forth in SEQ ID NO: 19. In another embodiment, the antibody or binding domain thereof comprises a light chain variable region having the amino acid sequences set forth in SEQ ID NO:20. In another embodiment, the antibody or binding domain thereof comprises heavy and light chain variable regions having the amino acid sequences set forth in SEQ ID NO: 19 and SEQ ID NO: 20, respectively.

An exemplary anti-PD-L1 antibody is antibody 7H7 as described herein. In one embodiment, the anti-PD-L1 antibody or binding domain thereof comprises the heavy and light chain CDRs or variable regions of antibody 7H7. In another embodiment, the antibody or binding domain thereof comprises the CDR1, CDR2, and CDR3 domains of the heavy chain variable region of antibody 7H7 having the sequence set forth in SEQ ID NO:77, and the CDR1, CDR2 and CDR3 domains of the light chain variable region of antibody 7H7 having the sequence set forth in SEQ ID NO:78. In another embodiment, the antibody or binding domain thereof comprises heavy chain CDR1, CDR2 and CDR3 domains having the sequences set forth in SEQ ID NOs: 29, 30, and 31, respectively, or conservative sequence modifications thereof, and light chain CDR1, CDR2 and CDR3 domains having the sequences set forth in SEQ ID NOs: 32, 33, and 34, respectively, or conservative sequence modifications thereof. In another embodiment, the antibody or binding domain thereof comprises a heavy chain variable region having the amino acid sequence set forth in SEQ ID NO: 77. In another embodiment, the antibody or binding domain thereof comprises a heavy chain variable region having the amino acid sequence set forth in SEQ ID NO:77. In another embodiment, the antibody or binding domain thereof comprises heavy and light chain variable regions having the amino acid sequences set forth in SEQ ID NO:77 and SEQ ID NO: 78, respectively.

Another exemplary anti-PD-L1 antibody is antibody 1B3 as described herein. In one embodiment, the anti-PD-L1 antibody or binding domain thereof comprises the heavy and light chain CDRs or variable regions of antibody 1B3. In another embodiment, the antibody or binding domain thereof comprises the CDR1, CDR2, and CDR3 domains of the heavy chain variable region of antibody 1B3 having the sequence set forth in SEQ ID NO:79, and the CDR1, CDR2 and CDR3 domains of the light chain variable region of antibody 1B3 having the sequence set forth in SEQ ID NO:80. In another embodiment, the antibody or binding domain thereof comprises heavy chain CDR1, CDR2 and CDR3 domains having the sequences set forth in SEQ ID NOs: 35, 36, and 37, respectively, or conservative sequence modifications thereof, and light chain CDR1, CDR2 and CDR3 domains having the sequences set forth in SEQ ID NOs: 38, 39, and 40, respectively, or conservative sequence modifications thereof. In another embodiment, the antibody or binding domain thereof comprises a heavy chain variable region having the amino acid sequences set forth in SEQ ID NO: 79. In another embodiment, the antibody or binding domain thereof comprises a light chain variable region having the amino acid sequences set forth in SEQ ID NO:80. In another embodiment, the antibody or binding domain thereof comprises heavy and light chain variable regions having the amino acid sequences set forth in SEQ ID NO:79 and SEQ ID NO: 80, respectively.

Another exemplary anti-PD-L1 antibody is antibody 3B6 as described herein. In one embodiment, the anti-PD-L1 antibody or binding domain thereof comprises the heavy and light chain CDRs or variable regions of antibody 3B6. In another embodiment, the antibody or binding domain thereof comprises the CDR1, CDR2, and CDR3 domains of the heavy chain variable region of antibody 3B6 having the sequence set forth in SEQ ID NO:81, and the CDR1, CDR2 and CDR3 domains of the light chain variable region of antibody 3B6 having the sequence set forth in SEQ ID NO:82. In another embodiment, the antibody or binding domain thereof comprises heavy chain CDR1, CDR2 and CDR3 domains having the sequences set forth in SEQ ID NOs: 41, 42, and 43, respectively, or conservative sequence modifications thereof, and light chain CDR1, CDR2 and CDR3 domains having the sequences set forth in SEQ ID NOs: 44, 45, and 46, respectively, or conservative sequence modifications thereof. In another embodiment, the antibody or binding domain thereof comprises a heavy chain variable region having the amino acid sequence set forth in SEQ ID NO: 81. In another embodiment, the antibody or binding domain thereof comprises a light chain variable region having the amino acid sequence set forth in SEQ ID NO:82. In another embodiment, the antibody or binding domain thereof comprises heavy and light chain variable regions having the amino acid sequences set forth in SEQ ID NO:81 and SEQ ID NO: 82, respectively.

Another exemplary anti-PD-L1 antibody is antibody 8B1 as described herein. In one embodiment, the anti-PD-L1 antibody or binding domain thereof comprises the heavy and light chain CDRs or variable regions of antibody 8B1. In another embodiment, the antibody or binding domain thereof comprises the CDR1, CDR2, and CDR3 domains of the heavy chain variable region of antibody 8B1 having the sequence set forth in SEQ ID NO:83, and the CDR1, CDR2 and CDR3 domains of the light chain variable region of antibody 8B1 having the sequence set forth in SEQ ID NO:84. In another embodiment, the antibody or binding domain thereof comprises heavy chain CDR1, CDR2 and CDR3 domains having the sequences set forth in SEQ ID NOs: 47, 48, and 49, respectively, or conservative sequence modifications thereof, and light chain CDR1, CDR2 and CDR3 domains having the sequences set forth in SEQ ID NOs: 50, 51, and 52, respectively, or conservative sequence modifications thereof. In another embodiment, the antibody or binding domain thereof comprises a heavy chain variable region having the amino acid sequences set forth in SEQ ID NO: 83. In another embodiment, the antibody or binding domain thereof comprises a light chain variable region having the amino acid sequences set forth in SEQ ID NO:84. In another embodiment, the antibody or binding domain thereof comprises heavy and light chain variable regions having the amino acid sequences set forth in SEQ ID NO:83 and SEQ ID NO: 84, respectively.

Another exemplary anti-PD-L1 antibody is antibody 4A3 as described herein. In one embodiment, the anti-PD-L1 antibody or binding domain thereof comprises the heavy and light chain CDRs or variable regions of antibody 4A3. In another embodiment, the antibody or binding domain thereof comprises the CDR1, CDR2, and CDR3 domains of the heavy chain variable region of antibody 4A3 having the sequence set forth in SEQ ID NO:85, and the CDR1, CDR2 and CDR3 domains of the light chain variable region of antibody 4A3 having the sequence set forth in SEQ ID NO:86. In another embodiment, the antibody or binding domain thereof comprises heavy chain CDR1, CDR2 and CDR3 domains having the sequences set forth in SEQ ID NOs: 53, 54, and 55, respectively, or conservative sequence modifications thereof, and light chain CDR1, CDR2 and CDR3 domains having the sequences set forth in SEQ ID NOs: 56, 57, and 58, respectively, or conservative sequence modifications thereof. In another embodiment, the antibody or binding domain thereof comprises a heavy chain variable region having the amino acid sequence set forth in SEQ ID NO: 85. In another embodiment, the antibody or binding domain thereof comprises a light chain variable region having the amino acid sequence set forth in SEQ ID NO:86. In another embodiment, the antibody or binding domain thereof comprises heavy and light chain variable regions having the amino acid sequences set forth in SEQ ID NO:85 and SEQ ID NO: 86, respectively.

Another exemplary anti-PD-L antibody is antibody 9H9 as described herein. In one embodiment, the anti-PD-L1 antibody or binding domain thereof comprises the heavy and light chain CDRs or variable regions of antibody 9H9. In another embodiment, the antibody or binding domain thereof comprises the CDR1, CDR2, and CDR3 domains of the heavy chain variable region of antibody 9H9 having the sequence set forth in SEQ ID NO:87, and the CDR1, CDR2 and CDR3 domains of the light chain variable region of antibody 9H9 having the sequence set forth in SEQ ID NO:88. In another embodiment, the antibody or binding domain thereof comprises heavy chain CDR1, CDR2 and CDR3 domains having the sequences set forth in SEQ ID NOs: 59, 60, and 61, respectively, or conservative sequence modifications thereof, and light chain CDR1, CDR2 and CDR3 domains having the sequences set forth in SEQ ID NOs: 62, 63, and 64, respectively, or conservative sequence modifications thereof. In another embodiment, the antibody or binding domain thereof comprises a heavy chain variable region having the amino acid sequence set forth in SEQ ID NO: 87. In another embodiment, the antibody or binding domain thereof comprises a light chain variable region having the amino acid sequence set forth in SEQ ID NO:88. In another embodiment, the antibody or binding domain thereof comprises heavy and light chain variable regions having the amino acid sequences set forth in SEQ ID NO:87 and SEQ ID NO: 88, respectively.

In one embodiment, the CDR1, 2, and/or 3 regions of the anti-C27 or anti-PD-L1 binding domains described herein can comprise the exact amino acid sequences as those of antibodies 3C2, 2B3, 7H7, 1B3, 3B6, 8B1, 4A3 and 9H9 disclosed herein. In another embodiment, the antibodies comprise derivatives from the exact CDR sequences of 3C2, 2B3, 7H7, 1B3, 3B6, 8B1, 4A3 and 9H9, yet still retain the ability of to bind either CD27 or PD-L1 effectively. Such sequence modifications may include one or more (e.g., 1, 2, 3, 4, 5, or 6) amino acid additions, deletions, or substitutions, e.g., conservative sequence modifications.

In another embodiment, the anti-C27 or anti-PD-L1 binding domains described herein can be composed of one or more CDRs that are, for example, 90%, 95%, 98% or 99.5% identical to one or more CDRs of antibodies 3C2, 2B3, 7H7, 1B3, 3B6, 8B1, 4A3 and 9H9. Ranges intermediate to the above-recited values, e.g., CDRs that are 90-95%, 95-98%, or 98-100% identical identity to one or more of the above sequences are also intended to be encompassed by the present invention.

The antibody sequences can also be consensus sequences of several antibodies. For example, in one embodiment, the anti-PD-L1 binding domain comprises a heavy chain variable region CDR1 comprising an amino acid sequence selected from the consensus sequence: (T,S)(S,Y,H)WMS (SEQ ID NO: 167). In another embodiment, the anti-PD-L1 binding domain comprises a heavy chain variable region CDR2 comprising SEQ ID NO:168. In another embodiment, the anti-PD-L1 binding domain comprises a heavy chain variable region CDR3 comprising SEQ ID NO:169. In another embodiment, the anti-PD-L1 binding domain comprises a light chain variable region CDR1 comprising SEQ ID NO:170. In another embodiment, the anti-PD-L1 binding domain comprises a light chain variable region CDR2 comprising SEQ ID NO: 171. In another embodiment, the anti-PD-L1 binding domain comprises a light chain variable region CDR3 comprising SEQ ID NO:172.

Sequences substantially identical to the anti-C27 and/or anti-PD-L1 binding domains described herein (e.g., at least 80%, 85%, 90%, 91%, 92%, 93%, 94%, 95%, 96%, 97%, 98% or 99% identical to the aforementioned sequences), are also encompassed by the invention. In one embodiment, the anti-CD27 binding domain comprises a heavy chain variable region comprising SEQ ID NO: 17, SEQ ID NO: 19 or a sequence at least 90% identical thereto (e.g., at least 90%, 91%, 92%, 93%, 94%, 95%, 96%, 97%, 98% or 99% identical to the aforementioned sequences). In another embodiment, the anti-CD27 binding domain comprises a light chain variable region comprising SEQ ID NO:18, SEQ ID NO:20, or a sequence at least 90% identical thereto (e.g., at least 90%, 91%, 92%, 93%, 94%, 95%, 96%, 97%, 98% or 99% identical to the aforementioned sequences). In another embodiment, the anti-CD27 binding domain comprises a heavy chain variable region comprising SEQ ID NO: 17 and a light chain variable region comprising SEQ ID NO: 18 or sequences at least 90% identical thereto (e.g., at least 90%, 91%, 92%, 93%, 94%, 95%, 96%, 97%, 98% or 99% identical to the aforementioned sequences). In another embodiment, the anti-CD27 binding domain comprises a heavy chain variable region comprising SEQ ID NO:19 and a light chain variable region comprising SEQ ID NO:20 or sequences at least 90% identical thereto (e.g., at least 90%, 91%, 92%, 93%, 94%, 95%, 96%, 97%, 98% or 99% identical to the aforementioned sequences).

In another embodiment, the anti-PD-L1 binding domain comprises a heavy chain variable region comprising SEQ ID NO:77, SEQ ID NO:79, SEQ ID NO:81, SEQ ID NO:83, SEQ ID NO:85, SEQ ID NO:87, or a sequence at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a light chain variable region comprising SEQ ID NO:78, SEQ ID NO:80, SEQ ID NO:82, SEQ ID NO:84, SEQ ID NO:86, SEQ ID NO:88 or a sequence at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:77 and a light chain variable region comprising SEQ ID NO: 78 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:79 and a light chain variable region comprising SEQ ID NO:80 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO: 81 and a light chain variable region comprising SEQ ID NO:82 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:83 and a light chain variable region comprising SEQ ID NO:84 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:85 and a light chain variable region comprising SEQ ID NO:86 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:87 and a light chain variable region comprising SEQ ID NO: 88 or sequences at least 90% identical thereto (e.g., at least 90%, 91%, 92%, 93%, 94%, 95%, 96%, 97%, 98% or 99% identical to the aforementioned sequences).

Anti-CD27 and/or anti-PD-L1 antibodies or binding domains thereof that compete for binding with any of the antibodies or binding domains thereof described herein or that bind the same epitope as any of the antibodies or binding domains thereof described herein are also suitable for use and are encompassed by the invention. For example, in one embodiment, the anti-CD27 antibody or binding domain thereof competes for binding to CD27 with antibody 3C2 and/or antibody 2B3, as described herein. For example, as described in Example 28, antibodies of the invention (e.g., antibody 2B3) bind to one or more residues within amino acids 80-95 of the ECD of human CD27 (SEQ ID NO: 183), e.g., one or more residues within amino acids 85-89, e.g., amino acids 85, 87, 88, and/or 89 of the ECD of human CD27 (SEQ ID NO: 183).

In another embodiment, the antibodies bind to the wildtype ECD of human CD27, but not to a mutated version of the ECD having amino acid substitutions at one or more positions within amino acid residues 85-89 (e.g., A85S, R87A, N88A, and/or G89A) of the ECD of human CD27 (SEQ ID NO: 183). For example, the anti-CD27 antibody, or antigen-binding fragment thereof, binds to the wildtype ECD of human CD27 (SEQ ID NO: 183), but does not bind to a mutated version of the wildtype ECD of human CD27 having the following amino acid substitutions: A85S, R87A, N88A, and G89A.

In another embodiment, the anti-CD27 antibody or binding domain binds to the same epitope on CD27 as antibody 3C2 and/or antibody 2B3, as described herein. In another embodiment, the antibody or anti-PD-L1 binding domain competes for binding to PD-L1 with antibody 7H7, 1B3, 3B6, 8B1, 4A3 and/or 9H9, as described herein. In another embodiment, the anti-PD-L1 antibody or binding domain binds to the same epitope on PD-L1 as antibody 7H7, 1B3, 3B6, 8B1, 4A3 and/or 9H9, as described herein.

In one aspect, a bispecific construct (or multispecific construct) comprising an anti-CD27 binding domain linked to an anti-PD-L1 binding domain is provided, wherein:
(i) the anti-CD27 binding domain comprises:
   a. heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof, or
   b. heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof; and
(ii) the anti-PD-L1 binding domain comprises:
   a. a heavy chain variable region CDR1 comprising an amino acid sequence selected from the consensus sequence: (T,S)(S,Y,H)WMS (SEQ ID NO: 167);
   b. a heavy chain variable region CDR2 comprising SEQ ID NO: 168;
   c. a heavy chain variable region CDR3 comprising SEQ ID NO: 169;
   d. a light chain variable region CDR1 comprising SEQ ID NO:170;
   e. a light chain variable region CDR2 comprising SEQ ID NO:171; and
   f. a light chain variable region CDR3 comprising SEQ ID NO:172.

In another embodiment, a bispecific construct comprising an anti-CD27 binding domain linked to an anti-PD-L1 binding domain, wherein:
(i) the anti-CD27 binding domain comprises:
   a. heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof, or
   b. heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof; and
(ii) the anti-PD-L1 binding domain comprises:
   a. a heavy chain variable region comprising SEQ ID NO:77 and a light chain variable region comprising SEQ ID NO:78 or sequences at least 95% identical thereto;
   b. a heavy chain variable region comprising SEQ ID NO:79 and a light chain variable region comprising SEQ ID NO:80 or sequences at least 95% identical thereto;
   c. a heavy chain variable region comprising SEQ ID NO:81 and a light chain variable region comprising SEQ ID NO:82 or sequences at least 95% identical thereto;
   d. a heavy chain variable region comprising SEQ ID NO:83 and a light chain variable region comprising SEQ ID NO:84 or sequences at least 95% identical thereto;
   e. a heavy chain variable region comprising SEQ ID NO:85 and a light chain variable region comprising SEQ ID NO:86 or sequences at least 95% identical thereto; or
   f. a heavy chain variable region comprising SEQ ID NO:87 and a light chain variable region comprising SEQ ID NO:88 or sequences at least 95% identical thereto.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof, and (b) an anti-PD-L1 binding domain comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:77 and a light chain variable region comprising SEQ ID NO:78.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof and (b) an anti-PD-L1 binding domain comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:79 and a light chain variable region comprising SEQ ID NO:80.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof and (b) an anti-PD-L1 binding domain comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:81 and a light chain variable region comprising SEQ ID NO:82.

In another embodiment, the bispecific construct comprises (a) an the anti-CD27 binding domain comprising heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof and (b) an anti-PD-L1 binding domain comprising heavy chain variable region CDR1, CDR2 and CDR3 as set forth in SEQ ID NOs: 47, 48, and 49, respectively, or conservative sequence modifications thereof, and light chain variable region CDR1, CDR2 and CDR3 as set forth in SEQ ID NOs: 50, 51, and 52, respectively, or conservative sequence modifications thereof.

In another embodiment, the bispecific construct comprises (a) an anti-CD27 binding domain comprising a heavy chain variable region comprising SEQ ID NO:17 and a light chain variable region comprising SEQ ID NO:18 and (b) an anti-PD-L1 binding domain comprising a heavy chain variable region comprising SEQ ID NO:83 and a light chain variable region comprising SEQ ID NO:84.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof, and (b) an anti-PD-L1 binding domain comprising heavy chain variable region CDR1, CDR2 and CDR3 as set forth in SEQ ID NOs: 53, 54, and 55, respectively, or conservative sequence modifications thereof, and light chain variable region CDR1, CDR2 and CDR3 as set forth in SEQ ID NOs: 56, 57, and 58, respectively, or conservative sequence modifications thereof.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:85 and a light chain variable region comprising SEQ ID NO:86.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof, and an anti-PD-L1 binding domain comprising heavy chain variable region CDR1, CDR2 and CDR3 as set forth in SEQ ID NOs: 59, 60, and 61, respectively, or conservative sequence modifications thereof, and light chain variable region CDR1, CDR2 and CDR3 as set forth in SEQ ID NOs: 62, 63, and 64, respectively, or conservative sequence modifications thereof.

In another embodiment, the bispecific construct comprises (a) an anti-CD27 binding domain comprising a heavy chain variable region comprising SEQ ID NO:17 and a light chain variable region comprising SEQ ID NO:18 and (b) an anti-PD-L1 binding domain comprising a heavy chain variable region comprising SEQ ID NO:87 and a light chain variable region comprising SEQ ID NO:88.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof, and (b) an anti-PD-L1 binding domain comprising heavy chain variable region CDR1, CDR2 and CDR3 as set forth in SEQ ID NOs: 29, 30, and 31, respectively, or conservative sequence modifications thereof, and light chain variable region CDR1, CDR2 and CDR3 as set forth in SEQ ID NOs: 32, 33, and 34, respectively, or conservative sequence modifications thereof.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprising a heavy chain variable region comprising SEQ ID NO:77 and a light chain variable region comprising SEQ ID NO:78.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof, and (b) an anti-PD-L1 binding domain comprising heavy chain variable region CDR1, CDR2 and CDR3 as set forth in SEQ ID NOs: 35, 36, and 37, respectively, or conservative sequence modifications thereof, and light chain variable region CDR1, CDR2 and CDR3 as set forth in SEQ ID NOs: 38, 39, and 40, respectively, or conservative sequence modifications thereof.

In another embodiment, the bispecific construct comprises (a) an anti-CD27 binding domain comprising a heavy chain variable region comprising SEQ ID NO:19 and a light chain variable region comprising SEQ ID NO:20 and (b) and anti-PD-L1 binding domain comprising a heavy chain variable region comprising SEQ ID NO:79 and a light chain variable region comprising SEQ ID NO:80.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof, and (b) an anti-PD-L1 binding domain comprising heavy chain variable region CDR1, CDR2 and CDR3 as set forth in SEQ ID NOs: 41, 42, and 43, respectively, or conservative sequence modifications thereof, and light chain variable region CDR1, CDR2 and CDR3 as set forth in SEQ ID NOs: 44, 45, and 46, respectively, or conservative sequence modifications thereof.

In another embodiment, the bispecific construct comprises (a) an anti-CD27 binding domain comprises a heavy chain variable region comprising SEQ ID NO: 19 and a light chain variable region comprising SEQ ID NO:20 and an anti-PD-L1 binding domain comprising a heavy chain variable region comprising SEQ ID NO:81 and a light chain variable region comprising SEQ ID NO:82.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof and (b) an anti-PD-L1 binding domain comprising heavy chain variable region CDR1, CDR2 and CDR3 as set forth in SEQ ID NOs: 47, 48, and 49, respectively, or conservative sequence modifications thereof, and light chain variable region CDR1, CDR2 and CDR3 as set forth in SEQ ID NOs: 50, 51, and 52, respectively, or conservative sequence modifications thereof.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprises a heavy chain variable region comprising SEQ ID NO:83 and a light chain variable region comprising SEQ ID NO:84.

In another embodiment, the bispecific construct comprises (a) an anti-CD27 binding domain comprising a heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof and (b) an anti-PD-L1 binding domain comprising a heavy chain variable region CDR1, CDR2 and CDR3 as set forth in SEQ ID NOs: 53, 54, and 55, respectively, or conservative sequence modifications thereof, and light chain variable region CDR1, CDR2 and CDR3 as set forth in SEQ ID NOs: 56, 57, and 58, respectively, or conservative sequence modifications thereof.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprising a heavy chain variable region comprising SEQ ID NO:85 and a light chain variable region comprising SEQ ID NO:86.

In another embodiment, the bispecific construct comprises (a) an anti-CD27 binding domain comprising a heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof, and (b) an anti-PD-L1 binding domain comprising a heavy chain variable region CDR1, CDR2 and CDR3 as set forth in SEQ ID NOs: 59, 60, and 61, respectively, or conservative sequence modifications thereof, and light chain variable region CDR1, CDR2 and CDR3 as set forth in SEQ ID NOs: 62, 63, and 64, respectively, or conservative sequence modifications thereof.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprises a heavy chain variable region comprising SEQ ID NO:87 and a light chain variable region comprising SEQ ID NO:88.

In one embodiment, the anti-PD-L1 binding domain and the anti-CD27 binding domain are genetically fused. The bispecific construct can be, for example, a fusion protein, which can be made by genetic engineering using standard recombinant DNA techniques to operatively link nucleic acid encoding the anti-CD27 and anti-PD-L1 binding domains. In another embodiment, the anti-PD-L1 binding domain and the anti-CD27 binding domain are chemically conjugated.

For example, the bispecific construct can be a chemical conjugate, which can be made by chemical conjugation of the anti-CD27 and anti-PD-L1 binding domains. In one embodiment, the anti-PD-L1 binding domain further comprises a human IgG1 constant domain. In another embodiment, the anti-CD27 binding domain is linked to the C-terminus of the heavy chain of the anti-PD-L1 binding domain. In another embodiment, the anti-CD27 binding domain is a scFv.

In another embodiment, the anti-CD27 binding domain further comprises a human IgG1 constant domain. In another embodiment, the anti-PD-L1 binding domain is linked to the C-terminus of the heavy chain of the anti-CD27 binding domain. In another embodiment, the anti-PD-L1 binding domain is a scFv.

In a particular embodiment, the bispecific construct comprises an anti-PD-L1 antibody linked to an anti-CD27 scFv, wherein:
(i) the anti-CD27 scFv comprises:
   a. heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, or
   b. heavy chain variable region CDR1, CDR2 and CDR3 as set forth in SEQ ID
      NOs: 7, 8, and 9, respectively, and light chain variable region CDR1, CDR2 and CDR3 as set forth in SEQ ID NOs: 10, 11, and 12, respectively; and
(ii) the anti-PD-L1 antibody comprises:
   a. heavy chain variable region CDR1, CDR2 and CDR3 as set forth in SEQ ID
      NOs: 29, 30, and 31, respectively, and light chain variable region CDR1, CDR2 and CDR3 as set forth in SEQ ID NOs: 32, 33, and 34, respectively;
   b. heavy chain variable region CDR1, CDR2 and CDR3 as set forth in SEQ ID
      NOs: 35, 36, and 37, respectively, and light chain variable region CDR1, CDR2 and CDR3 as set forth in SEQ ID NOs: 38, 39, and 40, respectively;
   c. heavy chain variable region CDR1, CDR2 and CDR3 as set forth in SEQ ID
      NOs: 41, 42, and 43, respectively, and light chain variable region CDR1, CDR2 and CDR3 as set forth in SEQ ID NOs: 44, 45, and 46, respectively;
   d. heavy chain variable region CDR1, CDR2 and CDR3 as set forth in SEQ ID
      NOs: 47, 48, and 49, respectively, and light chain variable region CDR1, CDR2 and CDR3 as set forth in SEQ ID NOs: 50, 51, and 52, respectively;
   e. heavy chain variable region CDR1, CDR2 and CDR3 as set forth in SEQ ID
      NOs: 53, 54, and 55, respectively, and light chain variable region CDR1, CDR2 and CDR3 as set forth in SEQ ID NOs: 56, 57, and 58, respectively;
or
   f. heavy chain variable region CDR1, CDR2 and CDR3 as set forth in SEQ ID
      NOs: 59, 60, and 61, respectively, and light chain variable region CDR1, CDR2 and CDR3 as set forth in SEQ ID NOs: 62, 63, and 64, respectively; and
   g. a human IgG1 constant domain.

In another particular embodiment, the bispecific construct comprises an anti-CD27 antibody linked to an anti-PD-L1 scFv, wherein:
(i) the anti-CD27 antibody comprises:
   a. heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, or
   b. heavy chain variable region CDR1, CDR2 and CDR3 as set forth in SEQ ID
      NOs: 7, 8, and 9, respectively, and light chain variable region CDR1, CDR2 and CDR3 as set forth in SEQ ID NOs: 10, 11, and 12, respectively; and
   c. a human IgG1 constant domain; and
(ii) the anti-PD-L1 scFv comprises:
   a. heavy chain variable region CDR1, CDR2 and CDR3 as set forth in SEQ ID
      NOs: 29, 30, and 31, respectively, and light chain variable region CDR1, CDR2 and CDR3 as set forth in SEQ ID NOs: 32, 33, and 34, respectively;
   b. heavy chain variable region CDR1, CDR2 and CDR3 as set forth in SEQ ID
      NOs: 35, 36, and 37, respectively, and light chain variable region CDR1, CDR2 and CDR3 as set forth in SEQ ID NOs: 38, 39, and 40, respectively;
   c. heavy chain variable region CDR1, CDR2 and CDR3 as set forth in SEQ ID
      NOs: 41, 42, and 43, respectively, and light chain variable region CDR1, CDR2 and CDR3 as set forth in SEQ ID NOs: 44, 45, and 46, respectively;
   d. heavy chain variable region CDR1, CDR2 and CDR3 as set forth in SEQ ID
      NOs: 47, 48, and 49, respectively, and light chain variable region CDR1, CDR2 and CDR3 as set forth in SEQ ID NOs: 50, 51, and 52, respectively;
   e. heavy chain variable region CDR1, CDR2 and CDR3 as set forth in SEQ ID
      NOs: 53, 54, and 55, respectively, and light chain variable region CDR1, CDR2 and CDR3 as set forth in SEQ ID NOs: 56, 57, and 58, respectively; or
   f. heavy chain variable region CDR1, CDR2 and CDR3 as set forth in SEQ ID
      NOs: 59, 60, and 61, respectively, and light chain variable region CDR1, CDR2 and CDR3 as set forth in SEQ ID NOs: 62, 63, and 64, respectively.

In another embodiment, the bispecific construct comprises an anti-PD-L1 antibody linked to an anti-CD27 scFv, wherein:
(i) the anti-CD27 scFv comprises:
   a. a heavy chain variable region comprising SEQ ID NO:17 and a light chain variable region comprising SEQ ID NO:18; or
   b. a heavy chain variable region comprising SEQ ID NO: 19 and a light chain variable region comprising SEQ ID NO:20; and
(ii) the anti-PD-L1 antibody comprises:
   a. a heavy chain variable region comprising SEQ ID NO:77 and a light chain variable region comprising SEQ ID NO:78;
   b. a heavy chain variable region comprising SEQ ID NO:79 and a light chain variable region comprising SEQ ID NO:80;
   c. a heavy chain variable region comprising SEQ ID NO:81 and a light chain variable region comprising SEQ ID NO:82;
   d. a heavy chain variable region comprising SEQ ID NO:83 and a light chain variable region comprising SEQ ID NO:84;
   e. a heavy chain variable region comprising SEQ ID NO:85 and a light chain variable region comprising SEQ ID NO:86; or
   f. a heavy chain variable region comprising SEQ ID NO:87 and a light chain variable region comprising SEQ ID NO:88; and
   g. a human IgG1 constant domain.

In another embodiment, the bispecific construct comprises an anti-CD27 antibody linked to an anti-PD-L1 scFv, wherein:
(i) the anti-CD27 antibody comprises:
   a. a heavy chain variable region comprising SEQ ID NO: 17 and a light chain variable region comprising SEQ ID NO:18; or
   b. a heavy chain variable region comprising SEQ ID NO:19 and a light chain variable region comprising SEQ ID NO:20; and
   c. a human IgG1 constant domain; and
(ii) the anti-PD-L1 scFv comprises:
   a. a heavy chain variable region comprising SEQ ID NO:77 and a light chain variable region comprising SEQ ID NO:78;
   b. a heavy chain variable region comprising SEQ ID NO:79 and a light chain variable region comprising SEQ ID NO:80;
   c. a heavy chain variable region comprising SEQ ID NO:81 and a light chain variable region comprising SEQ ID NO:82;
   d. a heavy chain variable region comprising SEQ ID NO:83 and a light chain variable region comprising SEQ ID NO:84;
   e. a heavy chain variable region comprising SEQ ID NO:85 and a light chain variable region comprising SEQ ID NO:86; or
   f. a heavy chain variable region comprising SEQ ID NO:87 and a light chain variable region comprising SEQ ID NO:88.

In another embodiment, the bispecific construct comprises an anti-PD-L1 antibody linked to an anti-CD27 scFv, wherein:
(i) the anti-CD27 scFv comprises heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and
   12, respectively; and
(ii) the anti-PD-L1 antibody comprises a heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively, and a human IgG1 constant domain.

In another embodiment, the bispecific construct comprises an anti-CD27 antibody linked to an anti-PD-L1 scFv, wherein:
(i) the anti-CD27 antibody comprises heavy chain variable region CDR1, CDR2 and
   CDR3 as set forth in SEQ ID NOs: 7, 8, and 9, respectively, and light chain variable region CDR1, CDR2 and CDR3 as set forth in SEQ ID NOs: 10, 11, and 12, respectively, and a human IgG1 constant domain; and
(ii) the anti-PD-L1 scFv comprises a heavy chain variable region CDR1, CDR2 and
   CDR3 as set forth in SEQ ID NOs: 47, 48, and 49, respectively, and light chain
   variable region CDR1, CDR2 and CDR3 as set forth in SEQ ID NOs: 50, 51, and
   52, respectively.

In another embodiment, the bispecific construct comprises an anti-PD-L1 antibody linked to an anti-CD27 scFv, wherein:
(i) the anti-CD27 scFv comprises a heavy chain variable region comprising SEQ ID NO: 19, a light chain variable region comprising SEQ ID NO:20; and
(ii) the anti-PD-L1 antibody comprises a heavy chain variable region comprising SEQ ID NO:83 and a light chain variable region comprising SEQ ID NO:84, and a human IgG1 constant domain.

In another embodiment, the bispecific construct comprises an anti-CD27 antibody linked to an anti-PD-L1 scFv, wherein:
(i) the anti-CD27 antibody comprises a heavy chain variable region comprising SEQ ID NO: 19, a light chain variable region comprising SEQ ID NO:20, and a human
   IgG1 constant domain; and
(ii) the anti-PD-L1 scFv comprises a heavy chain variable region comprising SEQ ID
   NO: 83 and a light chain variable region comprising SEQ ID NO:84.

In another embodiment, the bispecific construct comprises an anti-PD-L1 antibody linked to an anti-CD27 scFv, wherein:
(i) the anti-CD27 scFv comprises heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and
   12, respectively; and
(ii) the anti-PD-L1 antibody comprises heavy chain variable region CDR1, CDR2 and
   CDR3 as set forth in SEQ ID NOs: 59, 60, and 61, respectively, and light chain variable region CDR1, CDR2 and CDR3 as set forth in SEQ ID NOs: 62, 63, and 64, respectively, and a human IgG1 constant domain.

In another embodiment, the bispecific construct comprises an anti-CD27 antibody linked to an anti-PD-L1 scFv, wherein:
(i) the anti-CD27 antibody comprises heavy chain variable region CDR1, CDR2 and
   CDR3 as set forth in SEQ ID NOs: 7, 8, and 9, respectively, and light chain variable region CDR1, CDR2 and CDR3 as set forth in SEQ ID NOs: 10, 11, and
   12, respectively, and a human IgG1 constant domain; and
(ii) the anti-PD-L1 scFv comprises heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment, the bispecific construct comprises an anti-PD-L1 antibody linked to an anti-CD27 scFv, wherein:
(i) the anti-CD27 scFv comprises a heavy chain variable region comprising SEQ ID NO: 19, a light chain variable region comprising SEQ ID NO:20; and
(ii) the anti-PD-L1 antibody comprises a heavy chain variable region comprising SEQ ID NO:87 and a light chain variable region comprising SEQ ID NO:88, and a human IgG1 constant domain.

In another embodiment, the bispecific construct comprises an anti-CD27 antibody linked to an anti-PD-L1 scFv, wherein:
(i) the anti-CD27 antibody comprises a heavy chain variable region comprising SEQ
   ID NO: 19, a light chain variable region comprising SEQ ID NO:20, and a human IgG1 constant domain; and
(ii) the anti-PD-L1 scFv comprises a heavy chain variable region comprising SEQ ID
   NO: 87 and a light chain variable region comprising SEQ ID NO:88.

In another embodiment, the bispecific construct has one or more of the following functional features: induces NFκB activation, increases T cell proliferation, induces a CD8 T cell response, and/or increases IL-2 production. In another embodiment, the bispecific construct increases IL-2 production by at least about 1.5-fold (e.g., at least 2-fold, 2.5 fold, 3-fold, 3.5 fold, or 4-fold) compared to an anti-CD27 monoclonal antibody or anti-PD-L1 monoclonal antibody alone. In another embodiment, the bispecific construct induces a CD8 T cell response by at least about 2-fold greater (e.g., at least 2-fold, 2.5 fold, 3-fold, 3.5 fold, 4-fold, 4.5-fold, 5-fold, 5.5-fold, 6-fold, 6.5-fold, 7-fold, 7.5-fold, 8.0-fold, 8.5-fold, or 9-fold) than an anti-CD27 monoclonal antibody alone. In another embodiment, the bispecific construct increases survival by at least about 1.5-fold longer (e.g., at least 1.5-fold, 2.0-fold, 2.5 fold, 3-fold, 3.5 fold, 4-fold, 4.5-fold, or 5-fold) compared to an anti-CD27 monoclonal antibody or anti-PD-L1 monoclonal antibody alone. In another embodiment, the bispecific construct decreases tumor weight by at least about 1.5-fold (e.g., at least 1.5-fold, 2.0-fold, 2.5 fold, 3-fold, 3.5 fold, 4-fold, 4.5-fold, or 5-fold) compared to anti-CD27 monoclonal antibodies or anti-PD-L1 monoclonal antibodies alone or in combination. In another embodiment, the bispecific construct increases T cell production by at least about 1.5-fold (e.g., at least 1.5-fold, 2.0-fold, 2.5 fold, 3-fold, 3.5 fold, 4-fold, 4.5-fold, 5-fold, 5.5-fold, 6-fold, 6.5-fold, 7-fold, 7.5-fold, 8.0-fold, 8.5-fold, or 9-fold) compared to anti-CD27 monoclonal antibodies or anti-PD-L1 monoclonal antibodies alone or in combination.

In certain embodiments, the bispecific constructs described herein exhibit synergistic effects (e.g., in enhancing immune responses in vivo) as compared to use of anti-CD27 binding domains and anti-PD-L1 binding domains in combination (i.e., co-administration of unlinked antibodies).

In another aspect, novel anti-CD27 antibodies, or antigen-binding portions thereof, are provided which comprise any of the anti-CD27 binding domains described herein. In one embodiment, the anti-CD27 antibody, or antigen-binding fragment thereof, comprises heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively. In another embodiment, the anti-CD27 antibody, or antigen-binding fragment thereof, comprises a heavy chain variable region comprising SEQ ID NO: 17 and a light chain variable region comprising SEQ ID NO:18 or sequences at least 90% identical thereto (e.g., at least 90%, 91%, 92%, 93%, 94%, 95%, 96%, 97%, 98% or 99% identical to the aforementioned sequences).

In another embodiment, the anti-CD27 antibody, or antigen-binding fragment thereof, comprises heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively. In another embodiment, the anti-CD27 antibody, or antigen-binding fragment thereof, comprises a heavy chain variable region comprising SEQ ID NO: 19 and a light chain variable region comprising SEQ ID NO:20 or sequences at least 95% identical thereto.

In another embodiment, the anti-CD27 antibody, or antigen-binding fragment thereof, has one or more of the following functional features: induces or enhances a T cell-mediated immune response, blocks binding of sCD70 to CD27 (e.g., partially or completely), induces NFκB activation, increases T cell proliferation, binds to human CD27 with an equilibrium dissociation constant Kd of 10⁻⁹ M or less, or alternatively, an equilibrium association constant Ka of 10⁺⁹ M⁻¹ or greater, induces specific complement mediated cytotoxicity (CDC) of CD27 expressing cells, induces antibody dependent cell-mediated cytotoxicity (ADCC) specific lysis of CD27 expressing cells, induces or enhances antigen-specific immune responses in vivo in combination with a vaccine or endogenous antigen, induces or enhances antigen-specific TH1 immune responses in vivo in combination with a vaccine or endogenous antigen, induces or enhances antigen-specific T-cell proliferation or activation in vivo in combination with a vaccine or endogenous antigen; and/or induces or enhances T-cell activity when combined with simultaneous, separate or sequential TCR activation.

In another aspect, a bispecific construct is provided, wherein the bispecific construct comprises any one of the anti-CD27 antibodies described herein linked to an anti-PD-L1 binding domain. In one embodiment, the anti-PD-L1 binding domain is selected from the group consisting of:
a. an anti-PD-L1 antibody, or antigen binding fragment thereof, comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively;
b. an anti-PD-L1 antibody, or antigen binding fragment thereof, comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively;
c. an anti-PD-L1 antibody, or antigen binding fragment thereof, comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively;
d. an anti-PD-L1 antibody, or antigen binding fragment thereof, comprising heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively;
e. anti-PD-L1 antibody, or antigen binding fragment thereof, comprising heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively; and
f. anti-PD-L1 antibody, or antigen binding fragment thereof, comprising heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment, the anti-PD-L1 binding domain is selected from the group consisting of: (a) a heavy chain variable region comprising SEQ ID NO:77 and a light chain variable region comprising SEQ ID NO:78; (b) a heavy chain variable region comprising SEQ ID NO:79 and a light chain variable region comprising SEQ ID NO:80; (c) a heavy chain variable region comprising SEQ ID NO:81 and a light chain variable region comprising SEQ ID NO:82; (d) a heavy chain variable region comprising SEQ ID NO:83 and a light chain variable region comprising SEQ ID NO:84; (e) a heavy chain variable region comprising SEQ ID NO:85 and a light chain variable region comprising SEQ ID NO:86; and (f) a heavy chain variable region comprising SEQ ID NO:87 and a light chain variable region comprising SEQ ID NO:88. In a particular embodiment, the anti-PD-L1 binding domain is an scFv.

In certain embodiments, the bispecific constructs described herein exhibit synergistic effects (e.g., in enhancing immune responses in vivo) as compared to use of anti-CD27 binding domains and anti-PD-L1 binding domains in combination (i.e., co-administration of unlinked antibodies).

In another aspect, novel anti-PD-L1 antibodies, or antigen-binding portions thereof, are provided, which comprise any of the anti-PD-L1 binding domains described herein. In one embodiment, the anti-PD-L1 antibody, or antigen-binding fragment thereof, comprises heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively. In another embodiment, the anti-PD-L1 antibody, or antigen-binding fragment thereof, comprises a heavy chain variable region comprising SEQ ID NO: 77 and a light chain variable region comprising SEQ ID NO:78 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively. In another embodiment, the anti-PD-L1 antibody, or antigen-binding fragment thereof, comprises a heavy chain variable region comprising SEQ ID NO:79 and a light chain variable region comprising SEQ ID NO:80 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively. In another embodiment, the anti-PD-L1 antibody, or antigen-binding fragment thereof, comprises a heavy chain variable region comprising SEQ ID NO:81 and a light chain variable region comprising SEQ ID NO:82 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively. In another embodiment, the anti-PD-L1 antibody, or antigen-binding fragment thereof, comprises a heavy chain variable region comprising SEQ ID NO:83 and a light chain variable region comprising SEQ ID NO: 84 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively. In another embodiment, the anti-PD-L1 antibody, or antigen-binding fragment thereof, comprises a heavy chain variable region comprising SEQ ID NO: 85 and a light chain variable region comprising SEQ ID NO:86 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively. In another embodiment, the anti-PD-L1 antibody, or antigen-binding fragment thereof, comprises a heavy chain variable region comprising SEQ ID NO:87 and a light chain variable region comprising SEQ ID NO:88 or sequences at least 90% identical thereto (e.g., at least 90%, 91%, 92%, 93%, 94%, 95%, 96%, 97%, 98% or 99% identical to the aforementioned sequences).

In another embodiment, the anti-PD-L1 antibody, or antigen-binding fragment thereof, has one or more of the following functional features: (a) blocks binding of PD1 to PD-L1 (e.g., partially or completely), (b) induces NFAT pathway activation, and/or (c) induces a mixed lymphocyte response.

In another aspect, a bispecific construct is provided, wherein the bispecific construct comprises any one of the anti-PD-L1 antibodies, or antigen binding fragments thereof, described herein, linked to an anti-CD27 binding domain. In one embodiment, the anti-C27 binding domain comprises an anti-CD27 antibody comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively. In another embodiment, the anti-CD27 binding domain comprises an antibody comprising a heavy chain variable region comprising SEQ ID NO: 17 and a light chain variable region comprising SEQ ID NO: 18, or sequences at least 90% identical thereto (e.g., at least 90%, 91%, 92%, 93%, 94%, 95%, 96%, 97%, 98% or 99% identical to the aforementioned sequences). In another embodiment, the anti-C27 binding domain comprises an anti-CD27 antibody comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively. In another embodiment, the anti-C27 binding domain comprises an anti-CD27 antibody comprising a heavy chain variable region comprising SEQ ID NO: 19 and a light chain variable region comprising SEQ ID NO:20, or sequences at least 90% identical thereto (e.g., at least 90%, 91%, 92%, 93%, 94%, 95%, 96%, 97%, 98% or 99% identical to the aforementioned sequences). In one embodiment, the anti-CD27 binding domain further comprises a human IgG1 constant domain.

In certain embodiments, the bispecific constructs described herein exhibit synergistic effects (e.g., in enhancing immune responses in vivo) as compared to use of anti-CD27 binding domains and anti-PD-L1 binding domains in combination (i.e., co-administration of unlinked antibodies).

In another aspect, provided herein are compositions including any of the bispecific constructs (multispecific constructs), antibodies, or antigen binding fragments thereof, described herein and a pharmaceutically acceptable carrier. Also provided are kits comprising any of the bispecific constructs (multispecific constructs), antibodies, or antigen binding fragments thereof, described herein and instructions for use.

In a further aspect, isolated nucleic acid molecules encoding the binding domains, antibodies, or antigen-binding portions thereof, and bispecific or multispecific constructs described herein are also provided, as well as expression vectors comprising such nucleic acids and host cells comprising such expression vectors. In another embodiment, a nucleic acid molecule coding for any of the binding domains, antibodies, or antigen-binding portions thereof, or bispecific constructs described herein is provided. In another embodiment, the nucleic acid molecule is in the form of an expression vector. In another embodiment, the nucleic acid molecule is in the form of an expression vector which expresses the binding domain, antibody, or antigen-binding portion thereof, or bispecific construct when administered to a subject in vivo.

In one embodiment, the nucleic acid molecule comprises a nucleotide sequence encoding an antibody variable region, wherein the antibody variable region comprises the amino acid sequence depicted in SEQ ID NO: 17, 18, 19, 20, 77, 78, 79, 80, 81, 82, 83, 84, 85, 86, 87, 88, or an amino acid sequence at least 90% identical thereto (e.g., at least 90%, 91%, 92%, 93%, 94%, 95%, 96%, 97%, 98% or 99% identical to one or more of the aforementioned sequences). In another embodiment, the nucleic acid molecule comprises a nucleotide sequence as set forth in SEQ ID NO:25, 26, 27, 28, 101, 102, 103, 104, 105, 106, 107, 108, 109, 110, 111, 112, or a nucleotide sequence at least 90% identical thereto (e.g., at least 90%, 91%, 92%, 93%, 94%, 95%, 96%, 97%, 98% or 99% identical to one or more of the aforementioned sequences).

In another embodiment, the nucleic acid molecule comprises a nucleotide sequence encoding heavy and light chain variable regions of an antibody, wherein the heavy and light chain variable regions comprise the amino acid sequences depicted in SEQ ID NOs: 17 and 18, SEQ ID NOs: 19 and 20, SEQ ID NOs: 77 and 78, SEQ ID NOs: 79 and 80, SEQ ID NOs: 81 and 82, SEQ ID NOs: 83 and 84, SEQ ID NOs: 85 and 86, or SEQ ID NOs: 87 and 88, respectively, or amino acids sequences at least 90% identical thereto (e.g., at least 90%, 91%, 92%, 93%, 94%, 95%, 96%, 97%, 98% or 99% identical the aforementioned sequences).

In another aspect, methods of stimulating T cell activity are provided, which comprise contacting T cells with any one of the antibodies, or antigen binding fragments thereof, bispecific constructs, multispecific constructs, or the compositions of described herein. Stimulating T cell activity can comprise, for example, stimulating IFN-gamma production.

In yet another aspect, methods for inducing or enhancing an immune response (e.g., against an antigen) in a subject comprising administering to the subject any one of the antibodies, or antigen binding fragments thereof, bispecific constructs, multispecific constructs, or the compositions described herein, in an amount effective to induce or enhance an immune response in the subject (e.g., against an antigen).

In a further aspect, methods of for treating a condition or disease in a subject are provided, the method comprising administering to the subject any one of the antibodies, or antigen binding fragments thereof, bispecific constructs, multispecific constructs, or the compositions described herein, in an amount effective to treat the condition or disease.

In another aspect, methods for treating a condition or disease in a subject are provided, wherein the method comprises administering to the subject any one of the anti-CD27 antibodies, or antigen binding fragments thereof, described herein in combination with any one of the anti-PD-L1 antibodies, or antigen binding fragments thereof, described herein. For example, in one embodiment:
(i) the anti-CD27 antibody, or antigen binding fragment thereof, is selected from the group consisting of: (a) an anti-CD27 antibody, or antigen binding fragment thereof, comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, and (b) an anti-CD27 antibody, or antigen binding fragment thereof, comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively; and
(ii) the anti-PD-L1 antibody, or antigen binding fragment thereof, is selected from the group consisting of: (a) an anti-PD-L1 antibody, or antigen binding fragment thereof, comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively; (b) an anti-PD-L1 antibody, or antigen binding fragment thereof, comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively; (c) an anti-PD-L1 antibody, or antigen binding fragment thereof, comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively; (d) an anti-PD-L1 antibody, or antigen binding fragment thereof, comprising heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively; (e) anti-PD-L1 antibody, or antigen binding fragment thereof, comprising heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively; and (f) anti-PD-L1 antibody, or antigen binding fragment thereof, comprising heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment,
(i) the anti-CD27 antibody, or antigen binding fragment thereof, is selected from the group consisting of: (a) an anti-CD27 antibody, or antigen-binding fragment thereof, comprising a heavy chain variable region comprising SEQ ID NO:17 and a light chain variable region comprising SEQ ID NO:18 and (b) an anti-CD27 antibody, or antigen-binding fragment thereof, comprising SEQ ID NO: 19 and a light chain variable region comprising SEQ ID NO:20 or sequences; and
(ii) the anti-PD-L1 antibody, or antigen binding fragment thereof, is selected from the group consisting of: (a) an anti-PD-L1 antibody, or antigen binding fragment thereof, comprising a heavy chain variable region comprising SEQ ID NO:77 and a light chain variable region comprising SEQ ID NO:78; (b) an anti-PD-L1 antibody, or antigen binding fragment thereof, comprising a heavy chain variable region comprising SEQ ID NO: 79 and a light chain variable region comprising SEQ ID NO:80; (c) an anti-PD-L1 antibody, or antigen binding fragment thereof, comprising a heavy chain variable region comprising SEQ ID NO:81 and a light chain variable region comprising SEQ ID NO:82; (d) an anti-PD-L1 antibody, or antigen binding fragment thereof, comprising a heavy chain variable region comprising SEQ ID NO:83 and a light chain variable region comprising SEQ ID NO:84; (e) an anti-PD-L1 antibody, or antigen binding fragment thereof, comprising a heavy chain variable region comprising SEQ ID NO:85 and a light chain variable region comprising SEQ ID NO: 86; and (f) an anti-PD-L1 antibody, or antigen binding fragment thereof, comprising a heavy chain variable region comprising SEQ ID NO:87 and a light chain variable region comprising SEQ ID NO:88.

In one embodiment, the anti-CD27 antibody, or antigen binding fragment thereof, and the anti-PD-L1 antibody, or antigen binding fragment thereof, are administered separately. In one embodiment, the anti-CD27 antibody, or antigen binding fragment thereof, and the anti-PD-L1 antibody, or antigen binding fragment thereof, are administered sequentially. For example, the anti-CD27 antibody, or antigen binding fragment thereof, can be administered first followed by (e.g., immediately followed by) administration of the anti-PD-L1 antibody, or antigen binding fragment thereof, or vice versa. In another embodiment, the anti-CD27 antibody, or antigen binding fragment thereof, and the anti-PD-L1 antibody, or antigen binding fragment thereof, are administered together. In another embodiment, the anti-CD27 antibody, or antigen binding fragment thereof, and the anti-PD-L1 antibody, or antigen binding fragment thereof, are administered simultaneously. In another embodiment, the anti-CD27 antibody, or antigen binding fragment thereof, and the anti-PD-L1 antibody, or antigen binding fragment thereof, are simultaneously administered in a single formulation. Alternatively, the anti-CD27 antibody, or antigen binding fragment thereof, and the anti-PD-L1 antibody, or antigen binding fragment thereof, are formulated for separate administration and are administered concurrently or sequentially. Such concurrent or sequential administration preferably results in both antibodies being simultaneously present in treated patients.

In certain embodiments, administration of any of the anti-CD27 antibodies, or antigen binding fragment thereof, described herein in combination with any of the anti-PD-L1 antibodies, or antigen binding fragments thereof, described herein results in synergistic effects (e.g., in enhancing immune responses in vivo) as compared to use of either antibody alone.

The subject can be, for example, one who suffers from a condition or disease in which stimulation of an immune response is desired. In one embodiment, the condition or disease in which stimulation of an immune response is desired is cancer. The method of inducing or enhancing an immune response (e.g., against an antigen) in a subject can further comprise administering the antigen to the subject. Preferred antigens to be co-administered with the antibodies, or antigen binding fragments thereof, bispecific constructs, multispecific constructs, or the compositions of described herein are tumor antigens.

### III. BRIEF DESCRIPTION OF THE DRAWINGS

FIG. 1 is a graph showing binding of CD27 antibodies 2B3 and 3C2 to recombinant human CD27, as a function of antibody concentration.

FIG. 2 is a graph showing binding of CD27 antibodies 2B3 and 3C2 to recombinant cynomolgus CD27.

FIG. 3 is a graph showing high level binding of CD27 antibodies 2B3 and 3C2 to Ramos cells expressing CD27 on their surface.

FIG. 4 is a graph showing high level binding of CD27 antibodies 2B3 and 3C2 to T cells.

FIG. 5 is a graph showing antibodies 2B3 and 3C2 significantly block CD70 binding to CD27.

FIG. 6 is a graph showing antibodies 2B3 and 3C2 induced significant NFκB activation, as a function of antibody concentration.

FIG. 7 is a graph showing antibodies 2B3 and 3C2 increase T cell proliferation.

FIG. 8 is a graph showing anti-PD-L1 antibodies bind human PD-L1 as a function of antibody concentration.

FIG. 9 is a graph showing binding of anti-PD-L1 antibodies to recombinant cynomolgus PD-L1.

FIG. 10 is a graph showing anti-PD-L1 antibodies significantly block PD-L1 binding to PD1 as a function of antibody concentration.

FIG. 11 is a graph showing high level of binding of anti-PD-L1 antibodies to cells that express human PD-L1 as a function of antibody concentration.

FIG. 12 is a graph showing high level of binding of anti-PD-L1 antibodies to human dendritic cells as a function of antibody concentration.

FIG. 13 is a graph showing anti-PD-L1 antibodies block PD1/PD-L1 interaction between cells resulting in NFAT pathway activation.

FIG. 14 is a graph showing anti-PD-L1 antibodies induce a mixed lymphocyte response as a function of antibody concentration.

FIG. 15A is a representative DNA expression vector containing the anti-CD27 light chain, anti-CD27 heavy chain, and the c-terminal anti-PD-L1 single chain (VL+VH) peptide.

FIG. 15B is a drawing of the CD27/PD-L1 bispecific antibody protein wherein an anti-PD-L1 antibody is linked to an anti-CD27 scFv.

FIG. 15C is a drawing of the CD27/PD-L1 bispecific antibody protein wherein an anti-CD27 antibody is linked to an anti-PD-L1 scFv.

FIG. 15D is a table of representative anti-CD27/anti-PD-L1 bispecific constructs.

FIG. 15E shows characterization of the bispecific antibody CDX-527 by HPLC and gel electrophoresis.

FIG. 16 is a graph showing binding of anti-CD27/antiPD-L1 bispecific constructs (BsAbs) to CD27 and PD-L1 using a bifunctional ELISA.

FIG. 17 is a graph showing increased NFκB activation induced by anti-CD27/anti-PD-L1 bispecific constructs as compared to antibodies 1F5 or 2B3 alone.

FIG. 18 is a graph showing anti-CD27/anti-PD-L1 bispecific constructs block PD1/PD-L1 interaction and induce NFAT pathway activation as a function of antibody concentration.

FIG. 19 is a graph showing anti-CD27/anti-PD-L1 bispecific constructs increase IL-2 production/secretion in a mixed lymphocyte reaction as compared to antibodies AbX (a known anti-PD-L1 monoclonal antibody), 8B1, or 9H9 alone.

FIGS. 20A and 20B are graphs showing an anti-CD27/anti-PD-L1 bispecific construct (e.g., CD27xAbX) induces a higher CD8 T cell response as compared to CD27 monoclonal antibody alone.

FIG. 21 is a Kaplan-Meier curve showing improved survival of mice treated with a bispecific construct (e.g., CD27xAbX) compared to CD27 and PD-L1 antibodies administered alone or in combination in a mouse tumor model.

FIG. 22A is a graph showing decreased tumor weight in mice treated with a bispecific construct (e.g., CD27xAbX) compared to CD27 and PD-L1 antibodies administered alone or in combination. FIG. 22B is a graph showing increased percent CD8 T cells in mice treated with a bispecific construct (e.g., CD27xAbX) compared to CD27 and PD-L1 antibodies administered alone or in combination. FIG. 22C is a graph showing increased percent CD4 T cells in mice treated with a bispecific construct (e.g., CD27xAbX) compared to CD27 and PD-L1 antibodies administered alone or in combination. FIG. 22D is a graph showing increased activated CD8 T cells in mice treated with a bispecific construct (e.g., CD27xAbX) compared to CD27 and PD-L1 antibodies administered alone or in combination.

FIGS. 23A and 23B are graphs showing anti-CD27 Ab (e.g., CDX-1127) upregulates PD-L1 expression in tumor cells and tumor infiltrated cells.

FIG. 24 is a graph showing binding of the anti-CD27/antiPD-L1 bispecific construct CDX-527 to CD27 and PD-L1 using a bifunctional ELISA.

FIG. 25 are graphs showing increased NFκB activation induced by anti-CD27/anti-PD-L1 bispecific construct CDX-527 as compared to antibodies 1F5 or 2B3 alone (left graph) and also in presence of soluble FcγR1 (right graph).

FIG. 26 is a graph showing increased IL-2 production/secretion in a mixed lymphocyte reaction by anti-CD27/anti-PD-L1 bispecific construct CDX-527 as compared to antibodies 2B3 or 9H9 alone or in combination.

FIG. 27 is a graph showing increased IL-2 production/secretion in T-cells by anti-CD27/anti-PD-L1 bispecific construct CDX-527 as compared to antibodies 2B3 and 9H9 in combination.

FIG. 28 is a graph showing serum levels of CDX-527 in a NHP pharmacokinetic study.

FIG. 29 is a graph showing increased blockade of PD-1 signaling by the 9H9x2B3 configuration as compared to the 2B3x9H9 configuration.

FIG. 30 is a graph showing increased T-cell activation by the 9H9x2B3 configuration as compared to the 2B3x9H9 configuration.

FIG. 31 is a graph showing increased stimulation of a vaccine induced CD8+ T-cell response by AbXx2B3 as compared to 2B3xAbX.

FIG. 32 is a graph showing increased anti-tumor activity by AbXx2B3 as compared to 2B3xAbX.

FIG. 33 is a graph showing blocking of PD-L1 Binding to CD80 by anti-PD-L1 antibodies AbX and 9H9.

FIG. 34 (SEQ ID NO: 183) shows the sequence of the extracellular domain (ECD) of wild type huCD27 and a mutated version with substitutions at positions 85, 87, 88 and 89.

FIG. 35 is a graph showing binding of anti-CD27 antibody 2B3 to both wild type huCD27 and mutated huCD27 with sequences as shown in FIG. 34.

FIG. 36 is a graph showing improved production (expression) of a modified 9H9x2B3 (DD) construct compared to the original (unmodified) 9H9x2B3 construct.

### IV. DETAILED DESCRIPTION OF THE INVENTION

CD27 is an important co-stimulatory receptor that can be exploited for immunotherapy using agonist molecules. CD27 plays a key role in diverse immunological processes, including the survival, activation and effector functions of T cells, as well as the proliferation and cytotoxic activity of natural killer (NK) cells. These events occur in response to appropriate interaction of the ligand (CD70) with CD27 resulting in intracellular signaling events that lead to an activation of NF-κB and expression of relevant genes. As with most co-stimulatory molecules, effective stimulation of T cells with either the ligand or agonist antibodies also requires simultaneous stimulation through the T cell receptor (TCR).

Most CD27 agonists molecules require multimerization or crosslinking for their activity. For example, anti-CD27 antibodies that are agonists are cross-linked through interaction of their Fc domains with Fc receptors through cis or trans interactions. In vitro this can also be performed artificially using a secondary anti-Ig antibody or by adsorbing the antibody to a solid phase matrix. The requirement for FcR interaction of anti-CD27 agonist antibodies implies that the T cell stimulating activity is at least partially dependent on the number FcR expressing cells.

To eliminate the requirement for FcR interactions, CD27 agonists have been invented that 1) eliminate the need for interaction with receptors other than CD27 (herein termed CD27 hyper-crosslinking agents), or 2) can provide the crosslinking through interaction of alternate receptors that may be on different specific cell types not expressing FcR, and which alternate receptors may also provide additional function (herein termed CD27 multi-specific agents).

In one aspect, the invention provides bispecific constructs (or multispecific constructs) that comprise an anti-CD27 antibody, or antigen-binding fragment thereof, linked to an anti-PD-L1 antibody, or antigen-binding fragment thereof. Such anti-CD27×anti-PD-L1 bispecific agents of the invention have now been shown to exhibit synergistic effects in vivo, such as in enhancing immune parameters and inhibiting tumor growth, as compared to co-administration of an anti-CD27 antibody with an anti-PD-L1 antibody (see Examples 9 and 10).

In order that the present invention may be more readily understood, certain terms are first defined. Additional definitions are set forth throughout the detailed description.

### A. Definitions

As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

As used herein, the term “binding domain” refers to the portion of a protein or antibody which comprises the amino acid residues that interact with an antigen. Binding domains include, but are not limited to, antibodies (e.g., full length antibodies), as well as antigen-binding portions thereof. The binding domain confers on the binding agent its specificity and affinity for the antigen. The term also covers any protein having a binding domain which is homologous or largely homologous to an immunoglobulin-binding domain. Such proteins may be derived from natural sources, or partly or wholly synthetically produced.

The term “antibody” as referred to herein includes whole antibodies and any antigen binding fragment (i.e., “antigen-binding portion”) or single chain version thereof. An “antibody” refers, in one preferred embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody (or simply “antibody fragment”), as used herein, refers to one or more fragments or portions of an antibody that retain the ability to specifically bind to an antigen (e.g., human CD27). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody,” as used herein, refers to an antibody that displays a single binding specificity and affinity for a particular epitope. Accordingly, the term “human monoclonal antibody” refers to an antibody which displays a single binding specificity and which has variable and optional constant regions derived from human germline immunoglobulin sequences. In one embodiment, human monoclonal antibodies are produced by a hybridoma that includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23 (9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368 (6474): 856-859); Lonberg, N. (1994) Handbook of Experimental Pharmacology 113:49-101; Lonberg, N. and Huszar, D. (1995) Intern. Rev. Immunol. Vol. 13:65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., chimeric and humanized antibodies).

An “isolated antibody,” as used herein, is intended to refer to an antibody which is substantially free of other antibodies having different antigenic specificities (e.g., an isolated antibody that specifically binds to human CD27 is substantially free of antibodies that specifically bind antigens other than human CD27; an isolated antibody that specifically binds to human PD-L1 is substantially free of antibodies that specifically bind antigens other than human PD-L1). An isolated antibody that specifically binds to an epitope may, however, have cross-reactivity to the same antigen from different species. In addition, an isolated antibody is typically substantially free of other cellular material and/or chemicals.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from the antigen (e.g., CD27 or PD-L1) are tested for reactivity with the given antibody (e.g., an anti-CD27 or anti-PD-L1 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “antibody that binds the same epitope” as another antibody is intended to encompass antibodies that interact with, i.e., bind to, the same structural region on human CD27 as a reference anti-CD27 antibody. The “same epitope” to which the antibodies bind may be a linear epitope or a conformational epitope formed by tertiary folding of the antigen.

The term “competing antibody” refers to an antibody that competes for binding to human CD27 with a reference anti-CD27 antibody, i.e., competitively inhibits binding of the reference anti-CD27 antibody to CD27. A “competing antibody” may bind the same epitope on CD27 as the reference anti-CD27 antibody, may bind to an overlapping epitope or may sterically hinder the binding of the reference anti-CD27 antibody to CD27.

Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as CD27. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the terms “specific binding,” “selective binding,” “selectively binds,” and “specifically binds,” refer to antibody binding to an epitope on a predetermined antigen. Typically, the antibody binds with an equilibrium dissociation constant (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 2000 instrument (e.g., using recombinant human CD27 as the analyte and the antibody as the ligand) and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

The term “K_{D},” as used herein, is intended to refer to the dissociation equilibrium constant of a particular antibody-antigen interaction. Typically, the human antibodies of the invention bind to CD27 with a dissociation equilibrium constant (K_{D}) of approximately 10⁻⁸ M or less, such as less than 10⁻⁹ M or 10⁻¹⁰ M or even lower when determined by surface plasmon resonance (SPR) technology in a BIACORE 2000 instrument (e.g., using recombinant human CD27 as the analyte and the antibody as the ligand).

The term “kd” as used herein, is intended to refer to the off rate constant for the dissociation of an antibody from the antibody/antigen complex.

The term “ka” as used herein, is intended to refer to the on rate constant for the association of an antibody with the antigen.

The term “EC50,” as used herein, refers to the concentration of an antibody or an antigen-binding portion thereof, which induces a response, either in an in vitro or an in vivo assay, which is 50% of the maximal response, i.e., halfway between the maximal response and the baseline.

As used herein, “isotype” refers to the antibody class (e.g., IgM or IgG1) that is encoded by heavy chain constant region genes. In one embodiment, a human monoclonal antibody of the invention is of the IgG1 isotype. In another embodiment, a human monoclonal antibody of the invention is of the IgG2 isotype.

As used herein, the terms “inhibits” or “blocks” (e.g., referring to inhibition/blocking of binding of CD70 to CD27 and/or PD1 to PD-L1) are used interchangeably and encompass both partial and complete inhibition/blocking. The inhibition/blocking preferably reduces or alters the normal level or type of activity that occurs when binding occurs without inhibition or blocking. Inhibition and blocking are also intended to include any measurable decrease in the binding affinity of CD70 when in contact with an anti-CD27 antibody as compared to CD70 not in contact with an anti-CD27 antibody, e.g., inhibits binding of CD70 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In one embodiment, the anti-CD27 antibody inhibits binding of CD70 by at least about 70%. In another embodiment, the anti-CD27 antibody inhibits binding of CD70 by at least 80%. Inhibition and blocking are also intended to include any measurable decrease in the binding affinity of PD1 when in contact with an anti-PD-L1 antibody as compared to PD1 not in contact with an anti-PD-L1 antibody, e.g., inhibits binding of PD1 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In one embodiment, the anti-PD-L1 antibody inhibits binding of PD1 by at least about 70%. In another embodiment, the anti-PD-L1 antibody inhibits binding of PD1 by at least 80%.

The term “cross-reacts,” as used herein, refers to the ability of an anti-CD27 binding domain or an anti-PD-L1 binding domain of the invention to bind to CD27 or PD-L1, respectively, from a different species. For example, a CD27 binding domain of the invention that binds human CD27 may also bind another species of CD27. Similarly, an anti-PD-L1 binding domain of the invention that binds human PD-L1 may also bind another species of PD-L1. As used herein, cross-reactivity is measured by detecting a specific reactivity with purified antigen in binding assays (e.g., SPR, ELISA) or binding to, or otherwise functionally interacting with, cells physiologically expressing CD27. Methods for determining cross-reactivity include standard binding assays as described herein, for example, by Biacore™ surface plasmon resonance (SPR) analysis using a Biacore™ 2000 SPR instrument (Biacore AB, Uppsala, Sweden), or flow cytometric techniques.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The present invention also encompasses “conservative sequence modifications” of any of the sequences set forth in SEQ ID NOs: 1-160, i.e., nucleotide and amino acid sequence modifications which do not abrogate the binding of the VH and VL sequences encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs: 1-160 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD27 antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)).

In certain embodiments, conservative amino acid sequence modifications refer to at most 1, 2, 3, 4 or 5 conservative amino acid substitutions to the CDR sequences described herein. For example, each such CDR may contain up to 5 conservative amino acid substitutions, e.g., up to (i.e., not more than) 4 conservative amino acid substitutions, e.g., up to (i.e., not more than) 3 conservative amino acid substitutions, e.g., up to (i.e., not more than) 2 conservative amino acid substitutions, or no more than 1 conservative amino acid substitution.

Alternatively, in another embodiment, mutations can be introduced randomly along all or part of an anti-CD27 or anti-PD-L1 binding domain coding sequence, such as by saturation mutagenesis, and the resulting modified anti-CD27 or anti-PD-L1 antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For amino acids, the term “substantial homology” indicates that two amino acid sequences, or designated sequences thereof, when optimally aligned and compared, are identical, with appropriate amino acid insertions or deletions, in at least about 80% of the amino acids, usually at least about 90% to 95%, and more preferably at least about 98% to 99% or 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences identical to the nucleic acid molecules of the invention. BLAST protein searches can be performed with the XBLAST program, score=50, wordlength=3 to obtain amino acid sequences identical to the protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See http://www.ncbi.nlm.nih.gov.

### B. Anti-CD27 Antibodies and Binding Domains Thereof

Provided herein are novel anti-CD27 antibodies and binding domains thereof. The term “CD27” (also referred to as “CD27 molecule”, “CD27L receptor”, “S1521”, “T cell activation antigen CD27”, “TNFRSF7,” “MGC20393,” “tumor necrosis factor receptor superfamily, member 7”, “T cell activation antigen S152” “Tp55”, “Tumor necrosis factor receptor superfamily member 7”, “CD27 antigen”, and “T-cell activation antigen CD27”) refers to a receptor that is a member of the TNF-receptor superfamily, which binds to ligand CD70. CD27 is required for generation and long-term maintenance of T cell immunity and plays a key role in regulating B-cell activation and immunoglobulin synthesis. The term “CD27” includes any variants or isoforms of CD27 which are naturally expressed by cells (e.g., human CD27 deposited with GENBANK® having accession no. AAH12160.1 as set forth in SEQ ID NO: 173). Accordingly, CD27 binding domains of the invention may cross-react with CD27 from species other than human. Alternatively, the CD27 binding domains may be specific for human CD27 and may not exhibit any cross-reactivity with other species. CD27 or any variants and isoforms thereof, may either be isolated from cells or tissues that naturally express them or be recombinantly produced using well-known techniques in the art and/or those described herein. Preferably the CD27 binding domains are targeted to human CD27, which has a normal glycosylation pattern.

The term “CD70” (also referred to as “CD70 molecule”, “CD27L”, “CD27LG”, “TNFSF7,” “tumor necrosis factor (ligand) superfamily member 7,” “CD27 ligand,” “CD70 antigen,” “surface antigen CD70,” “tumor necrosis factor ligand superfamily, member 7,” “Ki-24 antigen,” and “CD27-L”) refers to the ligand for CD27 (see, for example, Bowman M R et al., J. Immunol. 1994 Feb. 15; 152 (4): 1756-61). CD70 is a type II transmembrane protein that belongs to the tumor necrosis factor (TNF) ligand family. It is a surface antigen on activated T and B lymphocytes that induces proliferation of co-stimulated T cells, enhances the generation of cytolytic T cells, and contributes to T cell activation. It has also been suggested that CD70 plays a role in regulating B-cell activation, cytotoxic function of natural killer cells, and immunoglobulin synthesis (Hintzen R Q et al., J. Immunol. 1994 Feb. 15; 152 (4): 1762-73). Genbank® Accession No. NP_001243 reports the amino acid sequence of human CD70 (SEQ ID NO:174).

An exemplary anti-CD27 antibody is antibody 3C2 as described herein. In one embodiment, the anti-CD27 antibody or binding domain thereof comprises the heavy and light chain CDRs or variable regions of antibody 3C2. In another embodiment, the antibody or binding domain thereof comprises the CDR1, CDR2, and CDR3 domains of the heavy chain variable region of antibody 3C2 having the sequence set forth in SEQ ID NO: 17, and the CDR1, CDR2 and CDR3 domains of the light chain variable region of antibody 3C2 having the sequence set forth in SEQ ID NO:18. In another embodiment, the antibody or binding domain thereof comprises heavy chain CDR1, CDR2 and CDR3 domains having the sequences set forth in SEQ ID NOs: 1, 2, and 3, respectively, or conservative sequence modifications thereof, and light chain CDR1, CDR2 and CDR3 domains having the sequences set forth in SEQ ID NOs: 4, 5, and 6, respectively, or conservative sequence modifications thereof. In another embodiment, the antibody or binding domain thereof comprises a heavy chain variable region having the amino acid sequence set forth in SEQ ID NO: 17. In another embodiment, the antibody or binding domain thereof comprises a light chain variable region having the amino acid sequence set forth in SEQ ID NO:18. In another embodiment, the antibody or binding domain thereof comprises heavy and light chain variable regions having the amino acid sequences set forth in SEQ ID NO:17 and SEQ ID NO: 18, respectively.

Another exemplary anti-CD27 antibody is antibody 2B3 as described herein. In one embodiment, the anti-CD27 antibody or binding domain thereof comprises the heavy and light chain CDRs or variable regions of antibody 2B3. In another embodiment, the antibody or binding domain thereof comprises the CDR1, CDR2, and CDR3 domains of the heavy chain variable region of antibody 2B3 having the sequence set forth in SEQ ID NO: 19, and the CDR1, CDR2 and CDR3 domains of the light chain variable region of antibody 3C2 having the sequence set forth in SEQ ID NO:20. In another embodiment, the antibody or binding domain thereof comprises heavy chain CDR1, CDR2 and CDR3 domains having the sequences set forth in SEQ ID NOs: 7, 8, and 9, respectively, or conservative sequence modifications thereof, and light chain CDR1, CDR2 and CDR3 domains having the sequences set forth in SEQ ID NOs: 10, 11, and 12, respectively, or conservative sequence modifications thereof. In another embodiment, the antibody or binding domain thereof comprises a heavy chain variable region having the amino acid sequences set forth in SEQ ID NO: 19. In another embodiment, the antibody or binding domain thereof comprises a light chain variable region having the amino acid sequences set forth in SEQ ID NO:20. In another embodiment, the antibody or binding domain thereof comprises heavy and light chain variable regions having the amino acid sequences set forth in SEQ ID NO: 19 and SEQ ID NO: 20, respectively.

Sequences substantially identical to the anti-C27 binding domains described herein (e.g., at least 80%, 85%, 90%, 91%, 92%, 93%, 94%, 95%, 96%, 97%, 98% or 99% identical (identical) to the aforementioned sequences), are also provided. In one embodiment, the anti-CD27 binding domain comprises a heavy chain variable region comprising SEQ ID NO:17, SEQ ID NO: 19 or a sequence at least 90% identical thereto (e.g., at least 90%, 91%, 92%, 93%, 94%, 95%, 96%, 97%, 98% or 99% identical to the aforementioned sequences). In another embodiment, the anti-CD27 binding domain comprises a light chain variable region comprising SEQ ID NO: 18, SEQ ID NO:20, or a sequence at least 90% identical thereto (e.g., at least 90%, 91%, 92%, 93%, 94%, 95%, 96%, 97%, 98% or 99% identical to the aforementioned sequences). In another embodiment, the anti-CD27 binding domain comprises a heavy chain variable region comprising SEQ ID NO:17 and a light chain variable region comprising SEQ ID NO: 18 or sequences at least 90% identical thereto (e.g., at least 90%, 91%, 92%, 93%, 94%, 95%, 96%, 97%, 98% or 99% identical to the aforementioned sequences). In another embodiment, the anti-CD27 binding domain comprises a heavy chain variable region comprising SEQ ID NO: 19 and a light chain variable region comprising SEQ ID NO: 20 or sequences at least 90% identical thereto (e.g., at least 90%, 91%, 92%, 93%, 94%, 95%, 96%, 97%, 98% or 99% identical to the aforementioned sequences).

Anti-CD27 antibodies and binding domains thereof that that compete for binding with any of the anti-CD27 antibody or binding domain thereof described herein or that bind the same epitope as any of the anti-CD27 antibody or binding domain thereof described herein are also suitable for use and provided herein. For example, in one embodiment, the anti-CD27 antibody or binding domain thereof competes for binding to CD27 with antibody 3C2 (or an antibody having the heavy and light chain CDRs and/or heavy and light chain variable region sequences corresponding to antibody 3C2). In another embodiment, the anti-CD27 antibody or binding domain thereof competes for binding to CD27 with antibody 2B3 (or an antibody having the heavy and light chain CDRs and/or heavy and light chain variable region sequences corresponding to antibody 2B3). In another embodiment, the antibody or anti-CD27 binding domain thereof binds to the same epitope on CD27 as antibody 3C2 (or an antibody having the heavy and light chain CDRs and/or heavy and light chain variable region sequences corresponding to antibody 3C2). In another embodiment, the anti-CD27 antibody or binding domain thereof binds to the same epitope on CD27 as antibody 2B3 (or an antibody having the heavy and light chain CDRs and/or heavy and light chain variable region sequences corresponding to antibody 2B3).

In one embodiment, the anti-CD27 antibody, or antigen-binding fragment thereof, comprises heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively. In another embodiment, the anti-CD27 antibody, or antigen-binding fragment thereof, comprises a heavy chain variable region comprising SEQ ID NO: 17 and a light chain variable region comprising SEQ ID NO:18 or sequences at least 90% identical thereto (e.g., at least 90%, 91%, 92%, 93%, 94%, 95%, 96%, 97%, 98% or 99% identical to the aforementioned sequences).

In another embodiment, the anti-CD27 antibody, or antigen-binding fragment thereof, comprises heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively. In another embodiment, the anti-CD27 antibody, or antigen-binding fragment thereof, comprises a heavy chain variable region comprising SEQ ID NO: 19 and a light chain variable region comprising SEQ ID NO:20 or sequences at least 95% identical thereto.

In another embodiment, the anti-CD27 antibody, or antigen-binding fragment thereof, has one or more of the following functional features: induces or enhances a T cell-mediated immune response, blocks binding of sCD70 to CD27 (e.g., partially or completely), induces NFκB activation, increases T cell proliferation, binds to human CD27 with an equilibrium dissociation constant Kd of 10⁻⁹ M or less, or alternatively, an equilibrium association constant Ka of 10⁺⁹ M⁻¹ or greater, induces specific complement mediated cytotoxicity (CDC) of CD27 expressing cells, induces antibody dependent cell-mediated cytotoxicity (ADCC) specific lysis of CD27 expressing cells, induces or enhances antigen-specific immune responses in vivo in combination with a vaccine or endogenous antigen, induces or enhances antigen-specific TH1 immune responses in vivo in combination with a vaccine or endogenous antigen, induces or enhances antigen-specific T-cell proliferation or activation in vivo in combination with a vaccine or endogenous antigen; and/or induces or enhances T-cell activity when combined with simultaneous, separate or sequential TCR activation.

### C. Anti-PD-L1 Antibodies and Binding Domains

Provided herein are novel anti-PD-L1 antibodies binding domains thereof. As used herein, the terms “Programmed cell death 1 ligand 1”, “PD-L1”, “PDCD1 ligand 1”, “Programmed death ligand 1”, “B7 homolog 1”, “B7-H1” and “CD274” are used interchangeably, and include variants, isoforms, species homologs of human PD-L1, and analogs having at least one common epitope with PD-L1. The complete PD-L1 sequence can be found under GenBank Accession No. NP_001254635 as set forth in SEQ ID NO:176.

Programmed death-ligand 1 (PD-L1) is a 40 kDa type 1 transmembrane protein that has been speculated to play a major role in suppressing the immune system during particular events such as pregnancy, tissue allografts, autoimmune disease, and other disease states such as hepatitis. Normally the immune system reacts to foreign antigens that are associated with exogenous or endogenous danger signals, which triggers a proliferation of antigen-specific CD8+ T cells and/or CD4+ helper cells. The binding of PD-L1 to PD-1 transmits an inhibitory signal that reduces the proliferation of these T cells and can also induce apoptosis, which is further mediated by a lower regulation of the gene Bcl-2. As used herein, the terms “Programmed Death 1,” “Programmed Cell Death 1,” “Protein PD-1,” “PD-1,” “PD1,” “PDCD1,” “hPD-1” and “hPD-I” are used interchangeably, and include variants, isoforms, species homologs of human PD-1, and analogs having at least one common epitope with PD-1. The complete PD-1 sequence can be found under GenBank Accession No. NP_005009 as set forth in SEQ ID NO:175.

PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

An exemplary anti-PD-L1 antibody is antibody 7H7 as described herein. In one embodiment, the anti-PD-L1 antibody or binding domain thereof comprises the heavy and light chain CDRs or variable regions of antibody 7H7. In another embodiment, the antibody or binding domain thereof comprises the CDR1, CDR2, and CDR3 domains of the heavy chain variable region of antibody 7H7 having the sequence set forth in SEQ ID NO:77, and the CDR1, CDR2 and CDR3 domains of the light chain variable region of antibody 7H7 having the sequence set forth in SEQ ID NO:78. In another embodiment, the antibody or binding domain thereof comprises heavy chain CDR1, CDR2 and CDR3 domains having the sequences set forth in SEQ ID NOs: 29, 30, and 31, respectively, or conservative sequence modifications thereof, and light chain CDR1, CDR2 and CDR3 domains having the sequences set forth in SEQ ID NOs: 32, 33, and 34, respectively, or conservative sequence modifications thereof. In another embodiment, the antibody or binding domain thereof comprises a heavy chain variable region having the amino acid sequence set forth in SEQ ID NO: 77. In another embodiment, the antibody or binding domain thereof comprises a heavy chain variable region having the amino acid sequence set forth in SEQ ID NO:77. In another embodiment, the antibody or binding domain thereof comprises heavy and light chain variable regions having the amino acid sequences set forth in SEQ ID NO:77 and SEQ ID NO: 78, respectively.

Another exemplary anti-PD-L1 antibody is antibody 1B3 as described herein. In one embodiment, the anti-PD-L1 antibody or binding domain thereof comprises the heavy and light chain CDRs or variable regions of antibody 1B3. In another embodiment, the antibody or binding domain thereof comprises the CDR1, CDR2, and CDR3 domains of the heavy chain variable region of antibody 1B3 having the sequence set forth in SEQ ID NO:79, and the CDR1, CDR2 and CDR3 domains of the light chain variable region of antibody 1B3 having the sequence set forth in SEQ ID NO:80. In another embodiment, the antibody or binding domain thereof comprises heavy chain CDR1, CDR2 and CDR3 domains having the sequences set forth in SEQ ID NOs: 35, 36, and 37, respectively, or conservative sequence modifications thereof, and light chain CDR1, CDR2 and CDR3 domains having the sequences set forth in SEQ ID NOs: 38, 39, and 40, respectively, or conservative sequence modifications thereof. In another embodiment, the antibody or binding domain thereof comprises a heavy chain variable region having the amino acid sequences set forth in SEQ ID NO: 79. In another embodiment, the antibody or binding domain thereof comprises a light chain variable region having the amino acid sequences set forth in SEQ ID NO:80. In another embodiment, the antibody or binding domain thereof comprises heavy and light chain variable regions having the amino acid sequences set forth in SEQ ID NO:79 and SEQ ID NO: 80, respectively.

Another exemplary anti-PD-L1 antibody is antibody 3B6 as described herein. In one embodiment, the anti-PD-L1 antibody or binding domain thereof comprises the heavy and light chain CDRs or variable regions of antibody 3B6. In another embodiment, the antibody or binding domain thereof comprises the CDR1, CDR2, and CDR3 domains of the heavy chain variable region of antibody 3B6 having the sequence set forth in SEQ ID NO:81, and the CDR1, CDR2 and CDR3 domains of the light chain variable region of antibody 3B6 having the sequence set forth in SEQ ID NO:82. In another embodiment, the antibody or binding domain thereof comprises heavy chain CDR1, CDR2 and CDR3 domains having the sequences set forth in SEQ ID NOs: 41, 42, and 43, respectively, or conservative sequence modifications thereof, and light chain CDR1, CDR2 and CDR3 domains having the sequences set forth in SEQ ID NOs: 44, 45, and 46, respectively, or conservative sequence modifications thereof. In another embodiment, the antibody or binding domain thereof comprises a heavy chain variable region having the amino acid sequence set forth in SEQ ID NO: 81. In another embodiment, the antibody or binding domain thereof comprises a light chain variable region having the amino acid sequence set forth in SEQ ID NO:82. In another embodiment, the antibody or binding domain thereof comprises heavy and light chain variable regions having the amino acid sequences set forth in SEQ ID NO:81 and SEQ ID NO: 82, respectively.

Another exemplary anti-PD-L1 antibody is antibody 8B1 as described herein. In one embodiment, the anti-PD-L1 antibody or binding domain thereof comprises the heavy and light chain CDRs or variable regions of antibody 8B1. In another embodiment, the antibody or binding domain thereof comprises the CDR1, CDR2, and CDR3 domains of the heavy chain variable region of antibody 8B1 having the sequence set forth in SEQ ID NO:83, and the CDR1, CDR2 and CDR3 domains of the light chain variable region of antibody 8B1 having the sequence set forth in SEQ ID NO:84. In another embodiment, the antibody or binding domain thereof comprises heavy chain CDR1, CDR2 and CDR3 domains having the sequences set forth in SEQ ID NOs: 47, 48, and 49, respectively, or conservative sequence modifications thereof, and light chain CDR1, CDR2 and CDR3 domains having the sequences set forth in SEQ ID NOs: 50, 51, and 52, respectively, or conservative sequence modifications thereof. In another embodiment, the antibody or binding domain thereof comprises a heavy chain variable region having the amino acid sequences set forth in SEQ ID NO: 83. In another embodiment, the antibody or binding domain thereof comprises a light chain variable region having the amino acid sequences set forth in SEQ ID NO:84. In another embodiment, the antibody or binding domain thereof comprises heavy and light chain variable regions having the amino acid sequences set forth in SEQ ID NO:83 and SEQ ID NO: 84, respectively.

Another exemplary anti-PD-L1 antibody is antibody 4A3 as described herein. In one embodiment, the anti-PD-L1 antibody or binding domain thereof comprises the heavy and light chain CDRs or variable regions of antibody 4A3. In another embodiment, the antibody or binding domain thereof comprises the CDR1, CDR2, and CDR3 domains of the heavy chain variable region of antibody 4A3 having the sequence set forth in SEQ ID NO:85, and the CDR1, CDR2 and CDR3 domains of the light chain variable region of antibody 4A3 having the sequence set forth in SEQ ID NO:86. In another embodiment, the antibody or binding domain thereof comprises heavy chain CDR1, CDR2 and CDR3 domains having the sequences set forth in SEQ ID NOs: 53, 54, and 55, respectively, or conservative sequence modifications thereof, and light chain CDR1, CDR2 and CDR3 domains having the sequences set forth in SEQ ID NOs: 56, 57, and 58, respectively, or conservative sequence modifications thereof. In another embodiment, the antibody or binding domain thereof comprises a heavy chain variable region having the amino acid sequence set forth in SEQ ID NO: 85. In another embodiment, the antibody or binding domain thereof comprises a light chain variable region having the amino acid sequence set forth in SEQ ID NO:86. In another embodiment, the antibody or binding domain thereof comprises heavy and light chain variable regions having the amino acid sequences set forth in SEQ ID NO:85 and SEQ ID NO: 86, respectively.

Another exemplary anti-PD-L1 antibody is antibody 9H9 as described herein. In one embodiment, the anti-PD-L1 antibody or binding domain thereof comprises the heavy and light chain CDRs or variable regions of antibody 9H9. In another embodiment, the antibody or binding domain thereof comprises the CDR1, CDR2, and CDR3 domains of the heavy chain variable region of antibody 9H9 having the sequence set forth in SEQ ID NO:87, and the CDR1, CDR2 and CDR3 domains of the light chain variable region of antibody 9H9 having the sequence set forth in SEQ ID NO:88. In another embodiment, the antibody or binding domain thereof comprises heavy chain CDR1, CDR2 and CDR3 domains having the sequences set forth in SEQ ID NOs: 59, 60, and 61, respectively, or conservative sequence modifications thereof, and light chain CDR1, CDR2 and CDR3 domains having the sequences set forth in SEQ ID NOs: 62, 63, and 64, respectively, or conservative sequence modifications thereof. In another embodiment, the antibody or binding domain thereof comprises a heavy chain variable region having the amino acid sequence set forth in SEQ ID NO: 87. In another embodiment, the antibody or binding domain thereof comprises a light chain variable region having the amino acid sequence set forth in SEQ ID NO:88. In another embodiment, the antibody or binding domain thereof comprises heavy and light chain variable regions having the amino acid sequences set forth in SEQ ID NO:87 and SEQ ID NO: 88, respectively.

The antibody sequences can also be consensus sequences of several antibodies. For example, in one embodiment, the anti-PD-L1 binding domain comprises a heavy chain variable region CDR1 comprising an amino acid sequence selected from the consensus sequence: (T,S)(S,Y,H)WMS (SEQ ID NO:167). In another embodiment, the anti-PD-L1 binding domain comprises a heavy chain variable region CDR2 comprising SEQ ID NO:168. In another embodiment, the anti-PD-L1 binding domain comprises a heavy chain variable region CDR3 comprising SEQ ID NO:169. In another embodiment, the anti-PD-L1 binding domain comprises a light chain variable region CDR1 comprising SEQ ID NO:170. In another embodiment, the anti-PD-L1 binding domain comprises a light chain variable region CDR2 comprising SEQ ID NO: 171. In another embodiment, the anti-PD-L1 binding domain comprises a light chain variable region CDR3 comprising SEQ ID NO:172.

Sequences substantially identical to the anti-PD-L1 antibodies and binding domains thereof described herein (e.g., at least 80%, 85%, 90%, 91%, 92%, 93%, 94%, 95%, 96%, 97%, 98% or 99% identical to the aforementioned sequences), are also encompassed by the invention. In one embodiment, the anti-PD-L1 binding domain comprises a heavy chain variable region comprising SEQ ID NO:77, SEQ ID NO:79, SEQ ID NO:81, SEQ ID NO:83, SEQ ID NO:85, SEQ ID NO:87, or a sequence at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a light chain variable region comprising SEQ ID NO:78, SEQ ID NO:80, SEQ ID NO:82, SEQ ID NO:84, SEQ ID NO:86, SEQ ID NO:88 or a sequence at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:77 and a light chain variable region comprising SEQ ID NO: 78 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:79 and a light chain variable region comprising SEQ ID NO:80 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO: 81 and a light chain variable region comprising SEQ ID NO:82 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:83 and a light chain variable region comprising SEQ ID NO:84 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:85 and a light chain variable region comprising SEQ ID NO:86 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 binding domain comprises a heavy chain variable region comprising SEQ ID NO:87 and a light chain variable region comprising SEQ ID NO: 88 or sequences at least 90% identical thereto (e.g., at least 90%, 91%, 92%, 93%, 94%, 95%, 96%, 97%, 98% or 99% identical to the aforementioned sequences).

Anti-PD-L1 antibodies and binding domains thereof that compete for binding with any of the anti-PD-L1 antibodies or binding domains thereof as described herein or that bind the same epitope as any of the anti-PD-L1 antibodies or binding domains thereof as described herein are also suitable for use and provided herein. For example, in one embodiment, the anti-PD-L1 antibody or binding domain thereof competes for binding to PD-L1 with antibody 7H7 (or an antibody having the heavy and light chain CDRs and/or heavy and light chain variable region sequences corresponding to antibody 7H7). In another embodiment, the anti-PD-L1 antibody or binding domain thereof binds to the same epitope on PD-L1 as antibody 7H7 (or an antibody having the heavy and light chain CDRs and/or heavy and light chain variable region sequences corresponding to antibody 7H7).

In another embodiment, the anti-PD-L1 antibody or binding domain thereof competes for binding to PD-L1 with antibody 1B3 (or an antibody having the heavy and light chain CDRs and/or heavy and light chain variable region sequences corresponding to antibody 1B3). In another embodiment, the anti-PD-L1 antibody or binding domain thereof binds to the same epitope on PD-L1 as antibody 1B3 (or an antibody having the heavy and light chain CDRs and/or heavy and light chain variable region sequences corresponding to antibody 1B3).

In another embodiment, the anti-PD-L1 antibody or binding domain thereof competes for binding to PD-L1 with antibody 3B6 (or an antibody having the heavy and light chain CDRs and/or heavy and light chain variable region sequences corresponding to antibody 3B6). In another embodiment, the anti-PD-L1 antibody or binding domain thereof binds to the same epitope on PD-L1 as antibody 3B6 (or an antibody having the heavy and light chain CDRs and/or heavy and light chain variable region sequences corresponding to antibody 3B6).

In another embodiment, the anti-PD-L1 antibody or binding domain thereof competes for binding to PD-L1 with antibody 8B1 (or an antibody having the heavy and light chain CDRs and/or heavy and light chain variable region sequences corresponding to antibody 8B1). In another embodiment, the anti-PD-L1 antibody or binding domain thereof binds to the same epitope on PD-L1 as antibody 8B1 (or an antibody having the heavy and light chain CDRs and/or heavy and light chain variable region sequences corresponding to antibody 8B1).

In another embodiment, the anti-PD-L1 antibody or binding domain thereof competes for binding to PD-L1 with antibody 4A3 (or an antibody having the heavy and light chain CDRs and/or heavy and light chain variable region sequences corresponding to antibody 4A3). In another embodiment, the anti-PD-L1 antibody or binding domain thereof binds to the same epitope on PD-L1 as antibody 4A3 (or an antibody having the heavy and light chain CDRs and/or heavy and light chain variable region sequences corresponding to antibody 4A3).

In another embodiment, the anti-PD-L1 antibody or binding domain thereof competes for binding to PD-L1 with antibody 9H9 (or an antibody having the heavy and light chain CDRs and/or heavy and light chain variable region sequences corresponding to antibody 9H9). In another embodiment, the anti-PD-L1 antibody or binding domain thereof binds to the same epitope on PD-L1 as antibody 9H9 (or an antibody having the heavy and light chain CDRs and/or heavy and light chain variable region sequences corresponding to antibody 9H9).

In another embodiment, the anti-PD-L1 binding domain is an anti-PD-L1 antibody, or antigen-binding portion thereof. In one embodiment, the anti-PD-L1 antibody, or antigen-binding fragment thereof, comprises heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively. In another embodiment, the anti-PD-L1 antibody, or antigen-binding fragment thereof, comprises a heavy chain variable region comprising SEQ ID NO:77 and a light chain variable region comprising SEQ ID NO:78 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively. In another embodiment, the anti-PD-L1 antibody, or antigen-binding fragment thereof, comprises a heavy chain variable region comprising SEQ ID NO:79 and a light chain variable region comprising SEQ ID NO: 80 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively. In another embodiment, the anti-PD-L1 antibody, or antigen-binding fragment thereof, comprises a heavy chain variable region comprising SEQ ID NO: 81 and a light chain variable region comprising SEQ ID NO:82 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively. In another embodiment, the anti-PD-L1 antibody, or antigen-binding fragment thereof, comprises a heavy chain variable region comprising SEQ ID NO:83 and a light chain variable region comprising SEQ ID NO:84 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively. In another embodiment, the anti-PD-L1 antibody, or antigen-binding fragment thereof, comprises a heavy chain variable region comprising SEQ ID NO:85 and a light chain variable region comprising SEQ ID NO:86 or sequences at least 90% identical thereto (e.g., at least 90%, 91%, 92%, 93%, 94%, 95%, 96%, 97%, 98% or 99% identical to the aforementioned sequences). In another embodiment, the anti-PD-L1 antibody, or antigen-binding fragment thereof, comprises heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively. In another embodiment, the anti-PD-L1 antibody, or antigen-binding fragment thereof, comprises a heavy chain variable region comprising SEQ ID NO:87 and a light chain variable region comprising SEQ ID NO: 88 or sequences at least 90% identical thereto (e.g., at least 90%, 91%, 92%, 93%, 94%, 95%, 96%, 97%, 98% or 99% identical to the aforementioned sequences).

In another embodiment, the anti-PD-L1 antibody, or antigen-binding fragment thereof, has one or more of the following functional features: (a) blocks binding of PD1 to PD-L1 (e.g., partially or completely), (b) induces NFAT pathway activation, and/or (c) induces a mixed lymphocyte reaction.

### D. Bispecific Constructs

Provided herein are bispecific constructs comprising an anti-CD27 binding domain linked to an anti-PD-L1 binding domain. Such bispecific constructs linked to one or more additional binding agents to form multispecific constructs also are provided.

A “bispecific” or “bifunctional” construct is an artificial hybrid having two different binding domain (e.g., heavy/light chain) pairs and two different binding sites. Bispecific constructs can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

For chemical conjugation, suitable reagents and methods are known in the art for coupling two or more moieties, in particular two or more antibodies, or fragments thereof, together. A variety of coupling or crosslinking agents are commercially available and can be used to conjugate the anti-CD27 binding domain and anti-PD-L1 binding domain. Non-limiting examples include Sulfo-SMCC, protein A, carboiimide, dimaleimide, dithio-bis-nitrobenzoic acid (DTNB), and N-succinimidyl-3-(2-pyridyldithio) propionate (SPDP). Sulfo-SMCC, SPDP and DTNB are preferred agents, with Sulfo-SMCC being particularly preferred. Other suitable procedures for crosslinking components (e.g., binding domains) with cross-linking agents are known in the art. See e.g., Karpovsky, B. et al., (1984) J. Exp. Med. 160:1686; Liu, M. A. et al., (1985) Proc. Natl. Acad. Sci USA 82:8648; Segal, D. M. and Perez, P., U.S. Pat. No. 4,676,980; and Brennan, M. (1986) Biotechniques 4:424.

For genetic engineering, nucleic acid molecules encoding the anti-CD27 binding domain can be inserted into an appropriate expression vector using standard recombinant DNA techniques. A nucleic acid molecule(s) encoding the anti-PD-L1 binding domain also can be inserted into the same expression vector, such that it is operatively linked (e.g., in-frame cloning) to the CD27 binding domain, thereby resulting in an expression vector that encodes a fusion protein that is the bispecific construct. Preferably, the anti-PD-L1 binding domain is operatively linked to the C-terminal region of the heavy chain of the anti-CD27 binding domain. Other suitable expression vectors and cloning strategies for preparing the bispecific constructs described herein are known in the art.

For expression of the bispecific constructs in host cells, the coding regions of the binding domains are combined with cloned promoter, leader sequence, translation initiation, leader sequence, constant region, 3′ untranslated, polyadenylation, and transcription termination, sequences to form expression vector constructs. These constructs can be used to express, for example, full length human IgG1K or IgG4K antibodies. Fully human, humanized and chimeric antibodies used in the bispecific constructs described herein also include IgG2, IgG3, IgE, IgA, IgM, and IgD antibodies. Similar plasmids can be constructed for expression of other heavy chain isotypes, or for expression of antibodies comprising lambda light chains.

Following preparation of an expression vector encoding the bispecific construct, the bispecific construct can be expressed recombinantly in a host cell using standard transfection methods. For example, in one embodiment, nucleic acid encoding the bispecific construct can be ligated into an expression vector, such as a eukaryotic expression plasmid, such as used by GS gene expression system disclosed in WO 87/04462, WO 89/01036 and EP 338 841 or other expression systems well known in the art. The purified plasmid with the cloned bispecific construct gene can be introduced in eukaryotic host cells, such as CHO-cells or NSO-cells or alternatively other eukaryotic cells like a plant derived cells, fungi or yeast cells. The method used to introduce these genes could be methods described in the art, such as electroporation, lipofectin, lipofectamine or other. After introducing the expression vector in the host cells, cells expressing the bispecific construct can be identified and selected. These cells represent the transfectomas that can then be amplified for their expression level and upscaled to produce bispecific constructs. Alternatively these cloned bispecific constructs can be expressed in other expression systems, such as E. coli or in complete organisms or can be synthetically expressed. Recombinant bispecific constructs can be isolated and purified from these culture supernatants and/or cells.

A bispecific construct of the invention, whether prepared by chemical conjugation or by genetic engineering, can be isolated and purified using one or more methodologies for protein purification well established in the art. Preferred methods for isolation and purification include, but are not limited to, gel filtration chromatography, affinity chromatography, anion-exchange chromatography and the like. A particularly preferred method is gel filtration chromatography, e.g., using a Superdex 200 column. Isolated and purified bispecific constructs can be evaluated using standard methods such as SDS-PAGE analysis.

Accordingly, in one embodiment, the anti-PD-L1 binding domain and the anti-CD27 binding domain are genetically fused. In another embodiment, the anti-PD-L1 binding domain and the anti-CD27 binding domain are chemically conjugated. In one embodiment, the anti-PD-L1 binding domain further comprises a human IgG1 constant domain. In another embodiment, the anti-CD27 binding domain is linked to the C-terminus of the heavy chain of the anti-PD-L1 binding domain. In another embodiment, the anti-CD27 binding domain is a scFv. In another embodiment, the anti-CD27 binding domain further comprises a human IgG1 constant domain. In another embodiment, the anti-PD-L1 binding domain is linked to the C-terminus of the heavy chain of the anti-CD27 binding domain. In another embodiment, the anti-PD-L1 binding domain is a scFv.

Exemplary bispecific constructs are set forth below in Tables 1-2, wherein the binding domains are defined by CDR sequences (Table 1) or variable region sequences (Table 2).

TABLE 1
Exemplary Bispecific Constructs (CDRs)

| Bispecific | CD27 Heavy Chain | CD27 Light Chain | PD-L1 Heavy Chain | PD-L1 Light Chain |
|---|---|---|---|---|
| Construct | Variable CDRs | Variable CDRs | Variable CDRs | Variable CDRs |
| 2B3x7H7 | CDR1: SEQ ID NO: 7 | CDR1: SEQ ID NO: 10 | CDR1: SEQ ID NO: 29 | CDR1: SEQ ID NO: 32 |
| | CDR2: SEQ ID NO: 8 | CDR2: SEQ ID NO: 11 | CDR2: SEQ ID NO: 30 | CDR2: SEQ ID NO: 33 |
| | CDR3: SEQ ID NO: 9 | CDR3: SEQ ID NO: 12 | CDR3: SEQ ID NO: 31 | CDR3: SEQ ID NO: 34 |
| 2B3x1B3 | CDR1: SEQ ID NO: 7 | CDR1: SEQ ID NO: 10 | CDR1: SEQ ID NO: 35 | CDR1: SEQ ID NO: 38 |
| | CDR2: SEQ ID NO: 8 | CDR2: SEQ ID NO: 11 | CDR2: SEQ ID NO: 36 | CDR2: SEQ ID NO: 39 |
| | CDR3: SEQ ID NO: 9 | CDR3: SEQ ID NO: 12 | CDR3: SEQ ID NO: 37 | CDR3: SEQ ID NO: 40 |
| 2B3x3B6 | CDR1: SEQ ID NO: 7 | CDR1: SEQ ID NO: 10 | CDR1: SEQ ID NO: 41 | CDR1: SEQ ID NO: 44 |
| | CDR2: SEQ ID NO: 8 | CDR2: SEQ ID NO: 11 | CDR2: SEQ ID NO: 42 | CDR2: SEQ ID NO: 45 |
| | CDR3: SEQ ID NO: 9 | CDR3: SEQ ID NO: 12 | CDR3: SEQ ID NO: 43 | CDR3: SEQ ID NO: 46 |
| 2B3x8B1 | CDR1: SEQ ID NO: 7 | CDR1: SEQ ID NO: 10 | CDR1: SEQ ID NO: 47 | CDR1: SEQ ID NO: 50 |
| | CDR2: SEQ ID NO: 8 | CDR2: SEQ ID NO: 11 | CDR2: SEQ ID NO: 48 | CDR2: SEQ ID NO: 51 |
| | CDR3: SEQ ID NO: 9 | CDR3: SEQ ID NO: 12 | CDR3: SEQ ID NO: 49 | CDR3: SEQ ID NO: 52 |
| 2B3x4A3 | CDR1: SEQ ID NO: 7 | CDR1: SEQ ID NO: 10 | CDR1: SEQ ID NO: 53 | CDR1: SEQ ID NO: 56 |
| | CDR2: SEQ ID NO: 8 | CDR2: SEQ ID NO: 11 | CDR2: SEQ ID NO: 54 | CDR2: SEQ ID NO: 57 |
| | CDR3: SEQ ID NO: 9 | CDR3: SEQ ID NO: 12 | CDR3: SEQ ID NO: 55 | CDR3: SEQ ID NO: 58 |
| 2B3x9H9 | CDR1: SEQ ID NO: 7 | CDR1: SEQ ID NO: 10 | CDR1: SEQ ID NO: 59 | CDR1: SEQ ID NO: 62 |
| | CDR2: SEQ ID NO: 8 | CDR2: SEQ ID NO: 11 | CDR2: SEQ ID NO: 60 | CDR2: SEQ ID NO: 63 |
| | CDR3: SEQ ID NO: 9 | CDR3: SEQ ID NO: 12 | CDR3: SEQ ID NO: 61 | CDR3: SEQ ID NO: 64 |
| 3C2x7H7 | CDR1: SEQ ID NO: 1 | CDR1: SEQ ID NO: 4 | CDR1: SEQ ID NO: 29 | CDR1: SEQ ID NO: 32 |
| | CDR2: SEQ ID NO: 2 | CDR2: SEQ ID NO: 5 | CDR2: SEQ ID NO: 30 | CDR2: SEQ ID NO: 33 |
| | CDR3: SEQ ID NO: 3 | CDR3: SEQ ID NO: 6 | CDR3: SEQ ID NO: 31 | CDR3: SEQ ID NO: 34 |
| 3C2x1B3 | CDR1: SEQ ID NO: 1 | CDR1: SEQ ID NO: 4 | CDR1: SEQ ID NO: 35 | CDR1: SEQ ID NO: 38 |
| | CDR2: SEQ ID NO: 2 | CDR2: SEQ ID NO: 5 | CDR2: SEQ ID NO: 36 | CDR2: SEQ ID NO: 39 |
| | CDR3: SEQ ID NO: 3 | CDR3: SEQ ID NO: 6 | CDR3: SEQ ID NO: 37 | CDR3: SEQ ID NO: 40 |
| 3C2x3B6 | CDR1: SEQ ID NO: 1 | CDR1: SEQ ID NO: 4 | CDR1: SEQ ID NO: 41 | CDR1: SEQ ID NO: 44 |
| | CDR2: SEQ ID NO: 2 | CDR2: SEQ ID NO: 5 | CDR2: SEQ ID NO: 42 | CDR2: SEQ ID NO: 45 |
| | CDR3: SEQ ID NO: 3 | CDR3: SEQ ID NO: 6 | CDR3: SEQ ID NO: 43 | CDR3: SEQ ID NO: 46 |
| 3C2x8B1 | CDR1: SEQ ID NO: 1 | CDR1: SEQ ID NO: 4 | CDR1: SEQ ID NO: 47 | CDR1: SEQ ID NO: 50 |
| | CDR2: SEQ ID NO: 2 | CDR2: SEQ ID NO: 5 | CDR2: SEQ ID NO: 48 | CDR2: SEQ ID NO: 51 |
| | CDR3: SEQ ID NO: 3 | CDR3: SEQ ID NO: 6 | CDR3: SEQ ID NO: 49 | CDR3: SEQ ID NO: 52 |
| 3C2x4A3 | CDR1: SEQ ID NO: 1 | CDR1: SEQ ID NO: 4 | CDR1: SEQ ID NO: 53 | CDR1: SEQ ID NO: 56 |
| | CDR2: SEQ ID NO: 2 | CDR2: SEQ ID NO: 5 | CDR2: SEQ ID NO: 54 | CDR2: SEQ ID NO: 57 |
| | CDR3: SEQ ID NO: 3 | CDR3: SEQ ID NO: 6 | CDR3: SEQ ID NO: 55 | CDR3: SEQ ID NO: 58 |
| 3C2x9H9 | CDR1: SEQ ID NO: 1 | CDR1: SEQ ID NO: 4 | CDR1: SEQ ID NO: 59 | CDR1: SEQ ID NO: 62 |
| | CDR2: SEQ ID NO: 2 | CDR2: SEQ ID NO: 5 | CDR2: SEQ ID NO: 60 | CDR2: SEQ ID NO: 63 |
| | CDR3: SEQ ID NO: 3 | CDR3: SEQ ID NO: 6 | CDR3: SEQ ID NO: 61 | CDR3: SEQ ID NO: 64 |

TABLE 2
Exemplary Bispecific Constructs (VRs)

| | CD27 | CD27 | PD-L1 | PD-L1 |
|---|---|---|---|---|
| Bispecific | Heavy Chain | Light Chain | Heavy Chain | Light Chain |
| Constructs | Variable Region | Variable Region | Variable Region | Variable Region |
| 2B3x7H7 | SEQ ID NO: 19 | SEQ ID NO: 20 | SEQ ID NO: 77 | SEQ ID NO: 78 |
| 2B3x1B3 | SEQ ID NO: 19 | SEQ ID NO: 20 | SEQ ID NO: 79 | SEQ ID NO: 80 |
| 2B3x3B6 | SEQ ID NO: 19 | SEQ ID NO: 20 | SEQ ID NO: 81 | SEQ ID NO: 82 |
| 2B3x8B1 | SEQ ID NO: 19 | SEQ ID NO: 20 | SEQ ID NO: 83 | SEQ ID NO: 84 |
| 2B3x4A3 | SEQ ID NO: 19 | SEQ ID NO: 20 | SEQ ID NO: 85 | SEQ ID NO: 86 |
| 2B3x9H9 | SEQ ID NO: 19 | SEQ ID NO: 20 | SEQ ID NO: 87 | SEQ ID NO: 88 |
| 3C2x7H7 | SEQ ID NO: 17 | SEQ ID NO: 18 | SEQ ID NO: 77 | SEQ ID NO: 78 |
| 3C2x1B3 | SEQ ID NO: 17 | SEQ ID NO: 18 | SEQ ID NO: 79 | SEQ ID NO: 80 |
| 3C2x3B6 | SEQ ID NO: 17 | SEQ ID NO: 18 | SEQ ID NO: 81 | SEQ ID NO: 82 |
| 3C2x8B1 | SEQ ID NO: 17 | SEQ ID NO: 18 | SEQ ID NO: 83 | SEQ ID NO: 84 |
| 3C2x4A3 | SEQ ID NO: 17 | SEQ ID NO: 18 | SEQ ID NO: 85 | SEQ ID NO: 86 |
| 3C2x9H9 | SEQ ID NO: 17 | SEQ ID NO: 18 | SEQ ID NO: 87 | SEQ ID NO: 88 |

In one embodiment, a bispecific construct comprising an anti-CD27 binding domain linked to an anti-PD-L1 binding domain is provided, wherein:
(i) the anti-CD27 binding domain comprises:
   a. heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, or
   b. heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively; and
(ii) the anti-PD-L1 binding domain comprises:
   a. a heavy chain variable region CDR1 comprising an amino acid sequence selected from the consensus sequence: (T,S)(S,Y,H)WMS (SEQ ID NO: 167);
   b. a heavy chain variable region CDR2 comprising SEQ ID NO: 168;
   c. a heavy chain variable region CDR3 comprising SEQ ID NO: 169;
   d. a light chain variable region CDR1 comprising SEQ ID NO:170;
   e. a light chain variable region CDR2 comprising SEQ ID NO:171; and
   f. a light chain variable region CDR3 comprising SEQ ID NO:172.

In another embodiment, a bispecific construct comprising an anti-CD27 binding domain linked to an anti-PD-L1 binding domain is provided, wherein:
(i) the anti-CD27 binding domain comprises:
   a. heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, or
   b. heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively; and
(ii) the anti-PD-L1 binding domain comprises:
   a. a heavy chain variable region comprising SEQ ID NO:77 and a light chain variable region comprising SEQ ID NO:78 or sequences at least 95% identical thereto (e.g., at least 95%, 96%, 97%, 98% or 99% identical to the aforementioned sequences);
   b. a heavy chain variable region comprising SEQ ID NO:79 and a light chain variable region comprising SEQ ID NO:80 or sequences at least 95% identical thereto (e.g., at least 95%, 96%, 97%, 98% or 99% identical to the aforementioned sequences);
   c. a heavy chain variable region comprising SEQ ID NO:81 and a light chain variable region comprising SEQ ID NO:82 or sequences at least 95% identical thereto (e.g., at least 95%, 96%, 97%, 98% or 99% identical to the aforementioned sequences);
   d. a heavy chain variable region comprising SEQ ID NO:83 and a light chain variable region comprising SEQ ID NO:84 or sequences at least 95% identical thereto (e.g., at least 95%, 96%, 97%, 98% or 99% identical to the aforementioned sequences);
   e. a heavy chain variable region comprising SEQ ID NO:85 and a light chain variable region comprising SEQ ID NO:86 or sequences at least 95% identical thereto (e.g., at least 95%, 96%, 97%, 98% or 99% identical to the aforementioned sequences); or
   f. a heavy chain variable region comprising SEQ ID NO:87 and a light chain variable region comprising SEQ ID NO:88 or sequences at least 95% identical thereto (e.g., at least 95%, 96%, 97%, 98% or 99% identical to the aforementioned sequences).

In another embodiment, the bispecific construct comprises (a) an anti-CD27 binding domain comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively and (b) an anti-PD-L1 binding domain comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:77 and a light chain variable region comprising SEQ ID NO:78.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively and (b) an anti-PD-L1 binding domain comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:79 and a light chain variable region comprising SEQ ID NO:80.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively and (b) an anti-PD-L1 binding domain comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:81 and a light chain variable region comprising SEQ ID NO:82.

In another embodiment, the bispecific construct comprises (a) an the anti-CD27 binding domain comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively and (b) an anti-PD-L1 binding domain comprising heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:83 and a light chain variable region comprising SEQ ID NO:84.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively; and (b) an anti-PD-L1 binding domain comprising heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively.

In another embodiment, the bispecific construct comprises (a) an anti-CD27 binding domain comprising a heavy chain variable region comprising SEQ ID NO: 17 and a light chain variable region comprising SEQ ID NO:18 and (b) an anti-PD-L1 binding domain comprising a heavy chain variable region comprising SEQ ID NO:85 and a light chain variable region comprising SEQ ID NO:86.

In another embodiment, the bispecific construct comprises (a) an anti-CD27 binding domain comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively and an anti-PD-L1 binding domain comprising heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment, the bispecific construct comprises (a) an anti-CD27 binding domain comprising a heavy chain variable region comprising SEQ ID NO:17 and a light chain variable region comprising SEQ ID NO:18 and (b) an anti-PD-L1 binding domain comprising a heavy chain variable region comprising SEQ ID NO:87 and a light chain variable region comprising SEQ ID NO:88.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively and (b) an anti-PD-L1 binding domain comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprising a heavy chain variable region comprising SEQ ID NO:77 and a light chain variable region comprising SEQ ID NO:78.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively and an anti-PD-L1 binding domain comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively.

In another embodiment, the bispecific construct comprises (a) an anti-CD27 binding domain comprising a heavy chain variable region comprising SEQ ID NO: 19 and a light chain variable region comprising SEQ ID NO:20 and (b) and anti-PD-L1 binding domain comprising a heavy chain variable region comprising SEQ ID NO:79 and a light chain variable region comprising SEQ ID NO:80.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively and an anti-PD-L1 binding domain comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively.

In another embodiment, the bispecific construct comprises (a) an anti-CD27 binding domain comprises a heavy chain variable region comprising SEQ ID NO: 19 and a light chain variable region comprising SEQ ID NO:20 and an anti-PD-L1 binding domain comprising a heavy chain variable region comprising SEQ ID NO:81 and a light chain variable region comprising SEQ ID NO:82.

In another embodiment, the bispecific construct comprises (a) an anti-CD27 binding domain comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively and (b) an anti-PD-L1 binding domain comprising heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprises a heavy chain variable region comprising SEQ ID NO:83 and a light chain variable region comprising SEQ ID NO:84.

In another embodiment, the bispecific construct comprises (a) an anti-CD27 binding domain comprising a heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively and (b) an anti-PD-L1 binding domain comprising a heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprising a heavy chain variable region comprising SEQ ID NO:85 and a light chain variable region comprising SEQ ID NO:86.

In another embodiment, the bispecific construct comprises (a) an anti-CD27 binding domain comprising a heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively and (b) an anti-PD-L1 binding domain comprising a heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment, the bispecific construct comprises (a) an anti-CD27 binding domain comprising a heavy chain variable region comprising SEQ ID NO: 19 and a light chain variable region comprising SEQ ID NO:20 and (b) an anti-PD-L1 binding domain comprises a heavy chain variable region comprising SEQ ID NO:87 and a light chain variable region comprising SEQ ID NO:88.

In a particular embodiment, the bispecific construct comprises an anti-PD-L1 antibody linked to an anti-CD27 scFv, wherein:
(i) the anti-CD27 scFv comprises:
   a. heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, or
   b. heavy chain variable region CDR1, CDR2 and CDR3 as set forth in SEQ ID
      NOs: 7, 8, and 9, respectively, and light chain variable region CDR1, CDR2 and CDR3 as set forth in SEQ ID NOs: 10, 11, and 12, respectively; and
(ii) the anti-PD-L1 antibody comprises:
   a. heavy chain variable region CDR1, CDR2 and CDR3 as set forth in SEQ ID
      NOs: 29, 30, and 31, respectively, and light chain variable region CDR1, CDR2 and CDR3 as set forth in SEQ ID NOs: 32, 33, and 34, respectively;
   b. heavy chain variable region CDR1, CDR2 and CDR3 as set forth in SEQ ID
      NOs: 35, 36, and 37, respectively, and light chain variable region CDR1, CDR2 and CDR3 as set forth in SEQ ID NOs: 38, 39, and 40, respectively;
   c. heavy chain variable region CDR1, CDR2 and CDR3 as set forth in SEQ ID
      NOs: 41, 42, and 43, respectively, and light chain variable region CDR1, CDR2 and CDR3 as set forth in SEQ ID NOs: 44, 45, and 46, respectively;
   d. heavy chain variable region CDR1, CDR2 and CDR3 as set forth in SEQ ID
      NOs: 47, 48, and 49, respectively, and light chain variable region CDR1, CDR2 and CDR3 as set forth in SEQ ID NOs: 50, 51, and 52, respectively;
   e. heavy chain variable region CDR1, CDR2 and CDR3 as set forth in SEQ ID
      NOs: 53, 54, and 55, respectively, and light chain variable region CDR1, CDR2 and CDR3 as set forth in SEQ ID NOs: 56, 57, and 58, respectively; or
   f. heavy chain variable region CDR1, CDR2 and CDR3 as set forth in SEQ ID
      NOs: 59, 60, and 61, respectively, and light chain variable region CDR1, CDR2 and CDR3 as set forth in SEQ ID NOs: 62, 63, and 64, respectively; and
   g. a human IgG1 constant domain.

In another particular embodiment, the bispecific construct comprises an anti-CD27 antibody linked to an anti-PD-L1 scFv, wherein:
(i) the anti-CD27 antibody comprises:
   a. heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, or
   b. heavy chain variable region CDR1, CDR2 and CDR3 as set forth in SEQ ID
      NOs: 7, 8, and 9, respectively, and light chain variable region CDR1, CDR2 and CDR3 as set forth in SEQ ID NOs: 10, 11, and 12, respectively; and
   c. a human IgG1 constant domain; and
(ii) the anti-PD-L1 scFv comprises:
   a. heavy chain variable region CDR1, CDR2 and CDR3 as set forth in SEQ ID
      NOs: 29, 30, and 31, respectively, and light chain variable region CDR1, CDR2 and CDR3 as set forth in SEQ ID NOs: 32, 33, and 34, respectively;
   b. heavy chain variable region CDR1, CDR2 and CDR3 as set forth in SEQ ID
      NOs: 35, 36, and 37, respectively, and light chain variable region CDR1, CDR2 and CDR3 as set forth in SEQ ID NOs: 38, 39, and 40, respectively;
   c. heavy chain variable region CDR1, CDR2 and CDR3 as set forth in SEQ ID
      NOs: 41, 42, and 43, respectively, and light chain variable region CDR1, CDR2 and CDR3 as set forth in SEQ ID NOs: 44, 45, and 46, respectively;
   d. heavy chain variable region CDR1, CDR2 and CDR3 as set forth in SEQ ID
      NOs: 47, 48, and 49, respectively, and light chain variable region CDR1, CDR2 and CDR3 as set forth in SEQ ID NOs: 50, 51, and 52, respectively;
   e. heavy chain variable region CDR1, CDR2 and CDR3 as set forth in SEQ ID
      NOs: 53, 54, and 55, respectively, and light chain variable region CDR1, CDR2 and CDR3 as set forth in SEQ ID NOs: 56, 57, and 58, respectively; or
   f. heavy chain variable region CDR1, CDR2 and CDR3 as set forth in SEQ ID
      NOs: 59, 60, and 61, respectively, and light chain variable region CDR1, CDR2 and CDR3 as set forth in SEQ ID NOs: 62, 63, and 64, respectively.

In another embodiment, the bispecific construct comprises an anti-PD-L1 antibody linked to an anti-CD27 scFv, wherein:
(i) the anti-CD27 scFv comprises:
   a. a heavy chain variable region comprising SEQ ID NO: 17 and a light chain variable region comprising SEQ ID NO:18; or
   b. a heavy chain variable region comprising SEQ ID NO: 19 and a light chain variable region comprising SEQ ID NO:20; and
(ii) the anti-PD-L1 antibody comprises:
   a. a heavy chain variable region comprising SEQ ID NO:77 and a light chain variable region comprising SEQ ID NO:78;
   b. a heavy chain variable region comprising SEQ ID NO:79 and a light chain variable region comprising SEQ ID NO:80;
   c. a heavy chain variable region comprising SEQ ID NO:81 and a light chain variable region comprising SEQ ID NO:82;
   d. a heavy chain variable region comprising SEQ ID NO:83 and a light chain variable region comprising SEQ ID NO:84;
   e. a heavy chain variable region comprising SEQ ID NO:85 and a light chain variable region comprising SEQ ID NO:86; or
   f. a heavy chain variable region comprising SEQ ID NO:87 and a light chain variable region comprising SEQ ID NO:88; and
   g. a human IgG1 constant domain.

In another embodiment, the bispecific construct comprises an anti-CD27 antibody linked to an anti-PD-L1 scFv, wherein:
(i) the anti-CD27 antibody comprises:
   a. a heavy chain variable region comprising SEQ ID NO: 17 and a light chain variable region comprising SEQ ID NO:18; or
   b. a heavy chain variable region comprising SEQ ID NO: 19 and a light chain variable region comprising SEQ ID NO:20; and
   c. a human IgG1 constant domain; and
(ii) the anti-PD-L1 scFv comprises:
   a. a heavy chain variable region comprising SEQ ID NO:77 and a light chain variable region comprising SEQ ID NO:78;
   b. a heavy chain variable region comprising SEQ ID NO:79 and a light chain variable region comprising SEQ ID NO:80;
   c. a heavy chain variable region comprising SEQ ID NO:81 and a light chain variable region comprising SEQ ID NO:82;
   d. a heavy chain variable region comprising SEQ ID NO:83 and a light chain variable region comprising SEQ ID NO:84;
   e. a heavy chain variable region comprising SEQ ID NO:85 and a light chain variable region comprising SEQ ID NO:86; or
   f. a heavy chain variable region comprising SEQ ID NO:87 and a light chain variable region comprising SEQ ID NO:88.

In another embodiment, the bispecific construct comprises an anti-PD-L1 antibody linked to an anti-CD27 scFv, wherein:
(i) the anti-CD27 scFv comprises heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively; and
(ii) the anti-PD-L1 antibody comprises a heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively, and a human IgG1 constant domain.

In another embodiment, the bispecific construct comprises an anti-CD27 antibody linked to an anti-PD-L1 scFv, wherein:
(i) the anti-CD27 antibody comprises heavy chain variable region CDR1, CDR2 and
   CDR3 as set forth in SEQ ID NOs: 7, 8, and 9, respectively, and light chain variable region CDR1, CDR2 and CDR3 as set forth in SEQ ID NOs: 10, 11, and
   12, respectively, and a human IgG1 constant domain; and
(ii) the anti-PD-L1 scFv comprises a heavy chain variable region CDR1, CDR2 and
   CDR3 as set forth in SEQ ID NOs: 47, 48, and 49, respectively, and light chain
   variable region CDR1, CDR2 and CDR3 as set forth in SEQ ID NOs: 50, 51, and
   52, respectively.

In another embodiment, the bispecific construct comprises an anti-PD-L1 antibody linked to an anti-CD27 scFv, wherein:
(i) the anti-CD27 scFv comprises a heavy chain variable region comprising SEQ ID NO: 19, a light chain variable region comprising SEQ ID NO:20; and
(ii) the anti-PD-L1 antibody comprises a heavy chain variable region comprising SEQ ID NO:83 and a light chain variable region comprising SEQ ID NO:84, and a human IgG1 constant domain.

In another embodiment, the bispecific construct comprises an anti-CD27 antibody linked to an anti-PD-L1 scFv, wherein:
(i) the anti-CD27 antibody comprises a heavy chain variable region comprising SEQ
   ID NO: 19, a light chain variable region comprising SEQ ID NO:20, and a human
   IgG1 constant domain; and
(ii) the anti-PD-L1 scFv comprises a heavy chain variable region comprising SEQ ID
   NO: 83 and a light chain variable region comprising SEQ ID NO:84.

In another embodiment, the bispecific construct comprises an anti-PD-L1 antibody linked to an anti-CD27 scFv, wherein:
(i) the anti-CD27 scFv comprises heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and
   12, respectively; and
(ii) the anti-PD-L1 antibody comprises heavy chain variable region CDR1, CDR2 and
   CDR3 as set forth in SEQ ID NOs: 59, 60, and 61, respectively, and light chain variable region CDR1, CDR2 and CDR3 as set forth in SEQ ID NOs: 62, 63, and 64, respectively, and a human IgG1 constant domain.

In another embodiment, the bispecific construct comprises an anti-CD27 antibody linked to an anti-PD-L1 scFv, wherein:
(i) the anti-CD27 antibody comprises heavy chain variable region CDR1, CDR2 and
   CDR3 as set forth in SEQ ID NOs: 7, 8, and 9, respectively, and light chain variable region CDR1, CDR2 and CDR3 as set forth in SEQ ID NOs: 10, 11, and
   12, respectively, and a human IgG1 constant domain; and
(ii) the anti-PD-L1 scFv comprises heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment, the bispecific construct comprises an anti-PD-L1 antibody linked to an anti-CD27 scFv, wherein:
(i) the anti-CD27 scFv comprises a heavy chain variable region comprising SEQ ID NO: 19, a light chain variable region comprising SEQ ID NO:20; and
(ii) the anti-PD-L1 antibody comprises a heavy chain variable region comprising SEQ ID NO:87 and a light chain variable region comprising SEQ ID NO:88, and a human IgG1 constant domain.

In another embodiment, the bispecific construct comprises an anti-CD27 antibody linked to an anti-PD-L1 scFv, wherein:
(i) the anti-CD27 antibody comprises a heavy chain variable region comprising SEQ
   ID NO: 19, a light chain variable region comprising SEQ ID NO:20, and a human IgG1 constant domain; and
(ii) the anti-PD-L1 scFv comprises a heavy chain variable region comprising SEQ ID
   NO: 87 and a light chain variable region comprising SEQ ID NO:88.

In another aspect, the bispecific construct is provided, wherein the bispecific construct comprises any one of the anti-CD27 antibodies, or antigen binding fragments thereof, described herein linked to an anti-PD-L1 binding domain. In one embodiment, the anti-PD-L1 binding domain is selected from the group consisting of:
a. an anti-PD-L1 antibody, or antigen binding fragment thereof, comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively;
b. an anti-PD-L1 antibody, or antigen binding fragment thereof, comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively;
c. an anti-PD-L1 antibody, or antigen binding fragment thereof, comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively;
d. an anti-PD-L1 antibody, or antigen binding fragment thereof, comprising heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively;
e. anti-PD-L1 antibody, or antigen binding fragment thereof, comprising heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively; and
f. anti-PD-L1 antibody, or antigen binding fragment thereof, comprising heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment, the anti-PD-L1 binding domain is selected from the group consisting of: (a) a heavy chain variable region comprising SEQ ID NO:77 and a light chain variable region comprising SEQ ID NO:78; (b) a heavy chain variable region comprising SEQ ID NO:79 and a light chain variable region comprising SEQ ID NO:80; (c) a heavy chain variable region comprising SEQ ID NO:81 and a light chain variable region comprising SEQ ID NO:82; (d) a heavy chain variable region comprising SEQ ID NO:83 and a light chain variable region comprising SEQ ID NO:84; (e) a heavy chain variable region comprising SEQ ID NO:85 and a light chain variable region comprising SEQ ID NO:86; and (f) a heavy chain variable region comprising SEQ ID NO:87 and a light chain variable region comprising SEQ ID NO:88. In a particular embodiment, the anti-PD-L1 binding domain further comprises a human IgG1 constant domain. In another embodiment, the anti-PD-L1 binding domain is an scFv.

In another aspect, a bispecific construct is provided, wherein the bispecific construct comprises any one of the anti-PD-L1 antibodies, or antigen binding fragments thereof, described herein, linked to an anti-CD27 binding domain. In one embodiment, the anti-C27 binding domain comprises an anti-CD27 antibody comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively. In another embodiment, the anti-CD27 binding domain comprises an antibody comprising a heavy chain variable region comprising SEQ ID NO:17 and a light chain variable region comprising SEQ ID NO: 18, or sequences at least 90% identical thereto (e.g., at least 90%, 91%, 92%, 93%, 94%, 95%, 96%, 97%, 98% or 99% identical to the aforementioned sequences). In another embodiment, the anti-C27 binding domain comprises an anti-CD27 antibody comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively. In another embodiment, the anti-C27 binding domain comprises an anti-CD27 antibody comprising a heavy chain variable region comprising SEQ ID NO: 19 and a light chain variable region comprising SEQ ID NO:20, or sequences at least 90% identical thereto (e.g., at least 90%, 91%, 92%, 93%, 94%, 95%, 96%, 97%, 98% or 99% identical to the aforementioned sequences). In one embodiment, the anti-CD27 binding domain is an scFv. In another embodiment, the anti-CD27 binding domain further comprises a human IgG1 constant domain.

In another embodiment, the bispecific construct has one or more of the following functional features: induces NFκB activation, increases T cell proliferation, induces a CD8 T cell response, and/or increases IL-2 production. In another embodiment, the bispecific construct increases IL-2 production by at least about 1.5-fold (e.g., at least 2-fold, 2.5 fold, 3-fold, 3.5 fold, or 4-fold) compared to an anti-CD27 monoclonal antibody or anti-PD-L1 monoclonal antibody alone. In another embodiment, the bispecific construct induces a CD8 T cell response by at least about 2-fold greater (e.g., at least 2-fold, 2.5 fold, 3-fold, 3.5 fold, 4-fold, 4.5-fold, 5-fold, 5.5-fold, 6-fold, 6.5-fold, 7-fold, 7.5-fold, 8.0-fold, 8.5-fold, or 9-fold) than an anti-CD27 monoclonal antibody alone. In another embodiment, the bispecific construct increases survival by at least about 1.5-fold longer (e.g., at least 1.5-fold, 2.0-fold, 2.5 fold, 3-fold, 3.5 fold, 4-fold, 4.5-fold, or 5-fold) compared to an anti-CD27 monoclonal antibody or anti-PD-L1 monoclonal antibody alone. In another embodiment, the bispecific construct decreases tumor weight by at least about 1.5-fold (e.g., at least 1.5-fold, 2.0-fold, 2.5 fold, 3-fold, 3.5 fold, 4-fold, 4.5-fold, or 5-fold) compared to anti-CD27 monoclonal antibodies or anti-PD-L1 monoclonal antibodies alone or in combination. In another embodiment, the bispecific construct increases T cell production by at least about 1.5-fold (e.g., at least 1.5-fold, 2.0-fold, 2.5 fold, 3-fold, 3.5 fold, 4-fold, 4.5-fold, 5-fold, 5.5-fold, 6-fold, 6.5-fold, 7-fold, 7.5-fold, 8.0-fold, 8.5-fold, or 9-fold) compared to anti-CD27 monoclonal antibodies or anti-PD-L1 monoclonal antibodies alone or in combination.

In certain embodiments, the bispecific constructs described herein exhibit synergistic effects (e.g., in enhancing immune responses in vivo) as compared to use of anti-CD27 binding domains and anti-PD-L1 binding domains in combination (i.e., co-administration of unlinked antibodies).

### E. Compositions

Also provided herein are compositions, e.g., a composition comprising one or a combination of any of the binding domains, antibodies, or antigen binding fragments thereof, the bispecific constructs, or the multispecific constructs described herein, formulated together with a carrier (e.g., a pharmaceutically acceptable carrier).

As used herein, the terms “carrier” and “pharmaceutically acceptable carrier” includes any and all solvents, salts, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound (i.e., any of the binding domains, antibodies, or antigen binding fragments thereof, the bispecific constructs, or the multispecific constructs described herein), may be coated in a material to protect the compound from the action of acids and other natural conditions that may inactivate the compound.

Examples of adjuvants which may be used with the binding domains, antibodies, or antigen binding fragments thereof, the bispecific constructs, or the multispecific constructs described here include, but are not limited to: Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatised polysaccharides; polyphosphazenes; biodegradable microspheres; cytokines, such as GM-CSF, interleukin-2, -7, -12, and other like factors; 3D-MPL; CpG oligonucleotide; and monophosphoryl lipid A, for example 3-de-O-acylated monophosphoryl lipid A.

MPL adjuvants are available from Corixa Corporation (Seattle, Wash; see, for example, U.S. Pat. Nos. 4,436,727; 4,877,611; 4,866,034 and 4,912,094). CpG-containing oligonucleotides (in which the CpG dinucleotide is unmethylated) are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996.

Further alternative adjuvants include, for example, saponins, such as Quil A, or derivatives thereof, including QS21 and QS7 (Aquila Biopharmaceuticals Inc., Framingham, Mass.); Escin; Digitonin; or Gypsophila or Chenopodium quinoa saponins; Montanide ISA 720 (Seppic, France); SAF (Chiron, California, United States); ISCOMS (CSL), MF-59 (Chiron); the SBAS series of adjuvants (e.g., SBAS-2 or SBAS-4, available from SmithKline Beecham, Rixensart, Belgium); Detox (Enhanzyn™) (Corixa, Hamilton, Mont.); RC-529 (Corixa, Hamilton, Mont.) and other aminoalkyl glucosaminide 4-phosphates (AGPs); polyoxyethylene ether adjuvants such as those described in WO 99/52549A1; synthetic imidazoquinolines such as imiquimod [S-26308, R-837], (Harrison, et al., Vaccine 19:1820-1826, 2001; and resiquimod [S-28463, R-848] (Vasilakos, et al., Cellular immunology 204: 64-74, 2000; Schiff bases of carbonyls and amines that are constitutively expressed on antigen presenting cell and T-cell surfaces, such as tucaresol (Rhodes, J. et al., Nature 377:71-75, 1995); cytokine, chemokine and co-stimulatory molecules as either protein or peptide, including for example pro-inflammatory cytokines such as Interferon, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L; immunostimulatory agents targeting ligands such as CTLA-4 and L-selectin, apoptosis stimulating proteins and peptides such as Fas; synthetic lipid based adjuvants, such as vaxfectin, (Reyes et al., Vaccine 19:3778-3786, 2001) squalene, alpha-tocopherol, polysorbate 80, DOPC and cholesterol; endotoxin, [LPS], (Beutler, B., Current Opinion in Microbiology 3:23-30, 2000); ligands that trigger Toll receptors to produce Th1-inducing cytokines, such as synthetic Mycobacterial lipoproteins, Mycobacterial protein p19, peptidoglycan, teichoic acid and lipid A; and CT (cholera toxin, subunits A and B) and LT (heat labile enterotoxin from E. coli, subunits A and B), heat shock protein family (HSPs), and LLO (listeriolysin O; WO 01/72329). These and various further Toll-like Receptor (TLR) agonists are described for example in Kanzler et al, Nature Medicine, May 2007, Vol 13, No 5.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Acceptable diluents include saline and aqueous buffer solutions. Liposomes include water-in-oil-in-water CGF emulsions as well as conventional liposomes (Strejan et al. (1984) J. Neuroimmunol. 7:27).

Carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the antibodies of the invention may be administered once or twice weekly by subcutaneous or intramuscular injection or once or twice monthly by subcutaneous or intramuscular injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

When the compounds of the present invention are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound that is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the tumor or infection. The composition must be fluid to the extent that easy syringability exists. It must be stable under the conditions of manufacture and storage and must be preserved against the contaminating action of microorganisms such as bacteria and fungi.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

### F. Nucleic Acids

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding binding domains, antibodies, or antibody portions (e.g., V_{H}, V_{L}, CDR3) that bind to CD27 and/or PD-L1, is intended to refer to a nucleic acid molecule in which the nucleotide sequences encoding the binding domain, antibodies, or antibody portions are free of other nucleotide sequences encoding the binding domain, antibodies, or antibody portions that bind antigens other than CD27 and/or PD-L1, which other sequences may naturally flank the nucleic acid in human genomic DNA.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid molecules of the present invention, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially identical to or derived from native V, D, J, constant, switches and other such sequences described herein are contemplated (where “derived” indicates that a sequence is identical or modified from another sequence).

A nucleic acid is “operably linked” or “operatively linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

Isolated nucleic acid molecules encoding the binding domains, antibodies, or antigen-binding portions thereof, bispecific constructs, and multispecific constructs described herein are also provided, as well as expression vectors comprising such nucleic acids and host cells comprising such expression vectors. In another embodiment, a nucleic acid molecule coding for any of the binding domains, antibodies, or antigen-binding portions thereof, bispecific constructs, or multispecific constructs described herein is provided. In another embodiment, the nucleic acid molecule is in the form of an expression vector. In another embodiment, the nucleic acid molecule is in the form of an expression vector which expresses the binding domain, antibody, or antigen-binding portion thereof, bispecific construct, or the multispecific construct when administered to a subject in vivo.

In one embodiment, the nucleic acid molecule comprises a nucleotide sequence encoding an antibody variable region, wherein the antibody variable region comprises the amino acid sequence depicted in SEQ ID NO:17, 18, 19, 20, 77, 78, 79, 80, 81, 82, 83, 84, 85, 86, 87, 88, or an amino acid sequence at least 90% identical thereto (e.g., at least 90%, 91%, 92%, 93%, 94%, 95%, 96%, 97%, 98% or 99% identical to one or more of the aforementioned sequences). In another embodiment, the nucleic acid molecule comprises a nucleotide sequence as set forth in SEQ ID NO:25, 26, 27, 28, 101, 102, 103, 104, 105, 106, 107, 108, 109, 110, 111, 112, or a nucleotide sequence at least 90% identical thereto (e.g., at least 90%, 91%, 92%, 93%, 94%, 95%, 96%, 97%, 98% or 99% identical to one or more of the aforementioned sequences).

In another embodiment, the nucleic acid molecule comprises a nucleotide sequence encoding heavy and light chain variable regions of an antibody, wherein the heavy and light chain variable regions comprise the amino acid sequences depicted in SEQ ID NOs: 17 and 18, SEQ ID NOs: 19 and 20, SEQ ID NOs: 77 and 78, SEQ ID NOs: 79 and 80, SEQ ID NOs: 81 and 82, SEQ ID NOs: 83 and 84, SEQ ID NOs: 85 and 86, or SEQ ID NOs: 87 and 88, respectively, or amino acids sequences at least 90% identical thereto (e.g., at least 90%, 91%, 92%, 93%, 94%, 95%, 96%, 97%, 98% or 99% identical the aforementioned sequences).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

### G. Combination Therapies

Any of the binding domains, antibodies, antigen binding fragments thereof, bispecific constructs, and/or multispecific constructs described herein, can be administered in combination with an additional therapy, i.e., combined with other agents. The term “coadministered” as used herein includes any or all of simultaneous, separate, or sequential administration of the binding domains, antibodies, antigen binding fragments thereof, bispecific constructs, or multispecific constructs described herein with adjuvants and other agents, including administration as part of a dosing regimen. For example, the combination therapy can include administering any of the binding domains, antibodies, antigen binding fragments thereof, bispecific constructs, and/or multispecific constructs described herein with at least one or more additional therapeutic agents, such as anti-inflammatory agents, DMARDs (disease-modifying anti-rheumatic drugs), immunosuppressive agents, chemotherapeutics, radiation therapy, other antibodies, cytotoxins and/or drugs, as well as adjuvants, immunostimulatory agents and/or immunosuppressive agents.

Chemotherapeutic agents suitable for coadministration with the binding domains, antibodies, antigen binding fragments thereof, bispecific constructs, and/or multispecific constructs described herein in the treatment of tumors include, for example: taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine) and temozolomide.

Agents that delete or inhibit immunosuppressive activities, for example, by immune cells (for example regulatory T-cells, NKT cells, macrophages, myeloid-derived suppressor cells, immature or suppressive dendritic cells) or suppressive factors produced by the tumor or host cells in the local microenvironment of the tumor (for example, TGFβ, indoleamine 2,3 dioxygenase-IDO), may also be administered with the binding domains, antibodies, antigen binding fragments thereof, bispecific constructs, and/or multispecific constructs described herein. Such agents include antibodies and small molecule drugs such as IDO inhibitors such as 1 methyl tryptophan or derivatives.

Suitable agents for coadministration with the binding domains, antibodies, antigen binding fragments thereof, bispecific constructs, and/or multispecific constructs described herein for treatment of such immune disorders include for example, immunosuppressive agents such as rapamycin, cyclosporin and FK506; anti-TNF agents such as etanercept, adalimumab and infliximab; and steroids. Examples of specific natural and synthetic steroids include, for example: aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol and triamcinolone.

Suitable agents for coadministration with the binding domains, antibodies, antigen binding fragments thereof, bispecific constructs, and/or multispecific constructs described herein for inducement or enhancement of an immune response include, for example, adjuvants and/or immunostimulatory agents, non-limiting examples of which have been disclosed hereinbefore. In one embodiment, the immunostimulatory agent is a TLR3 agonist, such as Poly IC.

As used herein, the term “immunostimulatory agent” includes, but is not limited to, compounds capable of stimulating antigen presenting cells (APCs), such as dendritic cells (DCs) and macrophages. For example, suitable immunostimulatory agents for use in the present invention are capable of stimulating APCs, so that the maturation process of the APCs is accelerated, the proliferation of APCs is increased, and/or the recruitment or release of co-stimulatory molecules (e.g., CD80, CD86, ICAM-1, MHC molecules and CCR7) and pro-inflammatory cytokines (e.g., IL-1B, IL-6, IL-12, IL-15, and IFN-γ) is upregulated. Suitable immunostimulatory agents are also capable of increasing T cell proliferation. Such immunostimulatory agents include, but are not be limited to, CD40 ligand; FLT 3 ligand; cytokines, such as IFN-α, IFN-β, IFN-γ and IL-2; colony-stimulating factors, such as G-CSF (granulocyte colony-stimulating factor) and GM-CSF (granulocyte-macrophage colony-stimulating factor); an anti-CTLA-4 antibody, anti-PD1 antibody, anti-41BB antibody, or anti-OX-40 antibody; LPS (endotoxin); ssRNA; dsRNA; Bacille Calmette-Guerin (BCG); Levamisole hydrochloride; and intravenous immune globulins. In one embodiment an immunostimulatory agant may be a Toll-like Receptor (TLR) agonist. For example the immunostimulatory agent may be a TLR3 agonist such as double-stranded inosine: cytosine polynucleotide (Poly I:C, for example available as Ampligen™ from Hemispherx Bipharma, PA, US or Poly IC:LC from Oncovir) or Poly A:U; a TLR4 agonist such as monophosphoryl lipid A (MPL) or RC-529 (for example as available from GSK, UK); a TLR5 agonist such as flagellin; a TLR7 or TLR8 agonist such as an imidazoquinoline TLR7 or TLR 8 agonist, for example imiquimod (eg Aldara™) or resiquimod and related imidazoquinoline agents (for example as available from 3M Corporation); or a TLR 9 agonist such as a deoxynucleotide with unmethylated CpG motifs (so-called “CpGs”, for example as available from Coley Pharmaceutical). Such immunostimulatory agents may be administered simultaneously, separately or sequentially with the binding domains, antibodies, antigen binding fragments thereof, bispecific constructs, and/or multispecific constructs described herein.

### H. Uses and Methods of the Invention

Provided herein are methods of stimulating T cell activity, methods of inducing or enhancing an immune response, and methods of treating a disease or condition (e.g., cancer) by administering the bispecific constructs, multispecific constructs, antibodies, or antigen binding fragments thereof, or compositions described herein to a patient in need thereof.

As used herein, the term “T cell-mediated response” or “T cell activity” refers to any response mediated by T cells, including effector T cells (e.g., CD8+ cells) and helper T cells (e.g., CD4+ cells). T cell mediated responses include, for example, T cell cytotoxicity and proliferation. Stimulation of T cell activity can be evaluated using any of a number of indicators of T cell activity known in the art. For example, enhancement of interferon-gamma production by OKT3-stimulated T cells can be used as a measure of T cell activation. Stimulation of T cell activity also can be evaluated using an NFκB-driven reporter gene system in a CD27-expressing cell. Other suitable assays of T cell activation are well established in the art.

The terms “inducing an immune response” and “enhancing an immune response” are used interchangeably and refer the stimulation of an immune response (i.e., either passive or adaptive) to a particular antigen.

The terms “treat,” “treating,” and “treatment,” as used herein, refer to therapeutic or preventative measures described herein. The methods of “treatment” employ administration to a subject, in need of such treatment, a bispecific construct, multispecific construct, antibody, antigen binding fragment thereof, or composition as described herein, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

The term “patient” includes human and other mammalian subjects that receive either prophylactic or therapeutic treatment.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%. In one aspect, methods of stimulating T cell activity are provided, which comprise contacting T cells with any one of the antibodies, or antigen binding fragments thereof, bispecific constructs, multispecific constructs, or the compositions of described herein. Stimulating T cell activity can comprise, for example, stimulating IFN-gamma production.

In another aspect, methods for inducing or enhancing an immune response (e.g., against an antigen) in a subject comprising administering to the subject any one of the antibodies, or antigen binding fragments thereof, bispecific constructs, multispecific constructs, or the compositions described herein, in an amount effective to induce or enhance an immune response in the subject (e.g., against an antigen).

In another aspect, methods of for treating a condition or disease in a subject are provided, the method comprising administering to the subject any one of the antibodies, or antigen binding fragments thereof, bispecific constructs, multispecific constructs, or the compositions described herein, in an amount effective to treat the condition or disease.

In another aspect, methods for treating a condition or disease in a subject are provided, wherein the method comprises administering to the subject any one of the anti-CD27 antibodies, or antigen binding fragments thereof, described herein in combination with any one of the anti-PD-L1 antibodies, or antigen binding fragments thereof, described herein. For example, in one embodiment:
(i) the anti-CD27 antibody, or antigen binding fragment thereof, is selected from the group consisting of: (a) an anti-CD27 antibody, or antigen binding fragment thereof, comprising heavy chain variable region CDR1, CDR2 and CDR3 as set forth in SEQ ID NOs: 1, 2, and 3, respectively, and light chain variable region CDR1, CDR2 and CDR3 as set forth in SEQ ID NOs: 4, 5, and 6, respectively, and (b) an anti-CD27 antibody, or antigen binding fragment thereof, comprising heavy chain variable region CDR1, CDR2 and CDR3 as set forth in SEQ ID NOs: 7, 8, and 9, respectively, and light chain variable region CDR1, CDR2 and CDR3 as set forth in SEQ ID NOs: 10, 11, and 12, respectively; and
(ii) the anti-PD-L1 antibody, or antigen binding fragment thereof, is selected from the group consisting of: (a) an anti-PD-L1 antibody, or antigen binding fragment thereof, comprising heavy chain variable region CDR1, CDR2 and CDR3 as set forth in SEQ ID NOs: 29, 30, and 31, respectively, and light chain variable region CDR1, CDR2 and CDR3 as set forth in SEQ ID NOs: 32, 33, and 34, respectively; (b) an anti-PD-L1 antibody, or antigen binding fragment thereof, comprising heavy chain variable region CDR1, CDR2 and CDR3 as set forth in SEQ ID NOs: 35, 36, and 37, respectively, and light chain variable region CDR1, CDR2 and CDR3 as set forth in SEQ ID NOs: 38, 39, and 40, respectively; (c) an anti-PD-L1 antibody, or antigen binding fragment thereof, comprising heavy chain variable region CDR1, CDR2 and CDR3 as set forth in SEQ ID NOs: 41, 42, and 43, respectively, and light chain variable region CDR1, CDR2 and CDR3 as set forth in SEQ ID NOs: 44, 45, and 46, respectively; (d) an anti-PD-L1 antibody, or antigen binding fragment thereof, comprising heavy chain variable region CDR1, CDR2 and CDR3 as set forth in SEQ ID NOs: 47, 48, and 49, respectively, and light chain variable region CDR1, CDR2 and CDR3 as set forth in SEQ ID NOs: 50, 51, and 52, respectively; (e) anti-PD-L1 antibody, or antigen binding fragment thereof, comprising heavy chain variable region CDR1, CDR2 and CDR3 as set forth in SEQ ID NOs: 53, 54, and 55, respectively, and light chain variable region CDR1, CDR2 and CDR3 as set forth in SEQ ID NOs: 56, 57, and 58, respectively; and (f) anti-PD-L1 antibody, or antigen binding fragment thereof, comprising heavy chain variable region CDR1, CDR2 and CDR3 as set forth in SEQ ID NOs: 59, 60, and 61, respectively, and light chain variable region CDR1, CDR2 and CDR3 as set forth in SEQ ID NOs: 62, 63, and 64, respectively.

In another embodiment,
(i) the anti-CD27 antibody, or antigen binding fragment thereof, is selected from the group consisting of: (a) an anti-CD27 antibody, or antigen-binding fragment thereof, comprising a heavy chain variable region comprising SEQ ID NO:17 and a light chain variable region comprising SEQ ID NO:18 and (b) an anti-CD27 antibody, or antigen-binding fragment thereof, comprising SEQ ID NO: 19 and a light chain variable region comprising SEQ ID NO:20 or sequences; and
(ii) the anti-PD-L1 antibody, or antigen binding fragment thereof, is selected from the group consisting of: (a) an anti-PD-L1 antibody, or antigen binding fragment thereof, comprising a heavy chain variable region comprising SEQ ID NO:77 and a light chain variable region comprising SEQ ID NO:78; (b) an anti-PD-L1 antibody, or antigen binding fragment thereof, comprising a heavy chain variable region comprising SEQ ID NO: 79 and a light chain variable region comprising SEQ ID NO:80; (c) an anti-PD-L1 antibody, or antigen binding fragment thereof, comprising a heavy chain variable region comprising SEQ ID NO:81 and a light chain variable region comprising SEQ ID NO:82; (d) an anti-PD-L1 antibody, or antigen binding fragment thereof, comprising a heavy chain variable region comprising SEQ ID NO:83 and a light chain variable region comprising SEQ ID NO:84; (e) an anti-PD-L1 antibody, or antigen binding fragment thereof, comprising a heavy chain variable region comprising SEQ ID NO:85 and a light chain variable region comprising SEQ ID NO: 86; and (f) an anti-PD-L1 antibody, or antigen binding fragment thereof, comprising a heavy chain variable region comprising SEQ ID NO:87 and a light chain variable region comprising SEQ ID NO:88.

In one embodiment, the anti-CD27 antibody, or antigen binding fragment thereof, and the anti-PD-L1 antibody, or antigen binding fragment thereof, are administered separately. In one embodiment, the anti-CD27 antibody, or antigen binding fragment thereof, and the anti-PD-L1 antibody, or antigen binding fragment thereof, are administered sequentially. For example, the anti-CD27 antibody, or antigen binding fragment thereof, can be administered first followed by (e.g., immediately followed by) administration of the anti-PD-L1 antibody, or antigen binding fragment thereof, or vice versa. In another embodiment, the anti-CD27 antibody, or antigen binding fragment thereof, and the anti-PD-L1 antibody, or antigen binding fragment thereof, are administered together. In another embodiment, the anti-CD27 antibody, or antigen binding fragment thereof, and the anti-PD-L1 antibody, or antigen binding fragment thereof, are administered simultaneously. In another embodiment, the anti-CD27 antibody, or antigen binding fragment thereof, and the anti-PD-L1 antibody, or antigen binding fragment thereof, are simultaneously administered in a single formulation. Alternatively, the anti-CD27 antibody, or antigen binding fragment thereof, and the anti-PD-L1 antibody, or antigen binding fragment thereof, are formulated for separate administration and are administered concurrently or sequentially. Such concurrent or sequential administration preferably results in both antibodies being simultaneously present in treated patients.

In certain embodiments, administration of any of the anti-CD27 antibodies, or antigen binding fragment thereof, described herein in combination with any of the anti-PD-L1 antibodies, or antigen binding fragments thereof, described herein results in synergistic effects (e.g., in enhancing immune responses in vivo) as compared to use of either antibody alone.

The subject can be, for example, one who suffers from a condition or disease in which stimulation of an immune response is desired. In one embodiment, the condition or disease is cancer. Types of cancers include, but are not limited to, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system. Particular cancers include CD27-expressing tumors selected from the group consisting of chronic lymphocytic leukemia, mantle cell lymphoma, primary central nervous system lymphoma, Burkitt's lymphoma and marginal zone B cell lymphoma.

Other disease indications include bacterial, fungal, viral and parasitic infectious diseases.

The methods of inducing or enhancing an immune response (e.g., against an antigen) in a subject described herein can further comprise administering the antigen to the subject. As used herein, the term “antigen” refers to any natural or synthetic immunogenic substance, such as a protein, peptide, hapten, polysaccharide and/or lipid. The bispecific construct, multispecific construct, antibody, antigen binding fragment thereof, or composition described herein and antigen can be administered at the same time or, alternatively, the bispecific construct, multispecific construct, antibody, antigen binding fragment thereof, or composition can be administered before or after the antigen is administered.

In one embodiment, a bispecific construct, multispecific construct, antibody, antigen binding fragment thereof, or composition described herein is administered in combination with a vaccine, to enhance the immune response against the vaccine antigen, for example a tumor antigen (to thereby enhance the immune response against the tumor) or an antigen from an infectious disease pathogen (to thereby enhance the immune response against the infectious disease pathogen). Accordingly, in one embodiment, a vaccine antigen can comprise, for example, an antigen or antigenic composition capable of eliciting an immune response against a tumor or against an infectious disease pathogen such as a virus, a bacteria, a parasite or a fungus. The antigen or antigens be derived from tumors, such as the various tumor antigens previously disclosed herein. Alternatively, the antigen or antigens can be derived from pathogens such as viruses, bacteria, parasites and/or fungi.

Preferred antigens to be co-administered with the antibodies, or antigen binding fragments thereof, bispecific constructs, multispecific constructs, or the compositions of described herein include tumor antigens and vaccine antigens (e.g., bacterial, viral or other pathogen antigens against which protective immunity is desired to be raised in a subject for purposes of vaccination). Additional examples of suitable pathogen antigens include tumor-associated antigens (TAAs), including but not limited to, sequences comprising all or part of the sequences of EGFR, EGFRvIII, gp100 or Pme117, HER2/neu, mesothelin, CEA, MARTI, MAGE-A1, MAGE-A2, MAGE-A3, MAGE-A4, MUC-1, GPNMB, HMW-MAA, TIM1, ROR1, CD19 and germ cell derived tumor antigens.

Other suitable antigens include viral antigens for the prevention or treatment of viral diseases. Examples of viral antigens include, but are not limited to, HIV-1 env, HBsAg, HPV, FAS, HSV-1, HSV-2, p17, ORF2 and ORF3 antigens. In addition, viral antigens or antigenic determinants can be derived from, for example, Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992, 10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Examples of bacterial antigens include, but are not limited to, Toxoplasma gondii or Treponema pallidum. The bacterial antigens can be in the treatment or prevention of various bacterial diseases such as Anthrax, Botulism, Tetanus, Chlamydia, Cholera, Diphtheria, Lyme Disease, Syphilis and Tuberculosis. Bacterial antigens or antigenic determinants can be derived from, for example: Bacillus spp., including B. anthracis (e.g., botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R. rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M. catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g., capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasitic/fungal antigens or antigenic determinants can be derived from, for example, Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.); Pneumocystis spp., including P. carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

It will be appreciated that in accordance with this aspect of the present invention antigens and antigenic determinants can be used in many different forms. For example, antigens or antigenic determinants can be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”), although it will be appreciated that the polynucleotides which can be used with this approach are not limited to DNA, and may also include RNA and modified polynucleotides as discussed above.

In one embodiment, a vaccine antigen can also be targeted, for example to particular cell types or to particular tissues. For example, the vaccine antigen can be targeted to Antigen Presenting Cells (APCs), for example by use of agents such as antibodies targeted to APC-surface receptors such as DEC-205, for example as discussed in WO 2009/061996 (Celldex Therapeutics, Inc), or the Mannose Receptor (CD206) for example as discussed in WO 03040169 (Medarex, Inc.).

### I. Kits

Also provided are kits (e.g., diagnostic kits) comprising one or more anti-CD27 binding domains, anti-PD-L1 binding domains, bispecific constructs, multispecific constructs, or compositions as described herein, optionally with instructions for use. Kits may also include informative pamphlets, for example, pamphlets informing one how to use reagents to practice a method disclosed herein. The term “pamphlet” includes any writing, marketing materials or recorded material supplied on or with the kit, or which otherwise accompanies the kit.

The present invention is further illustrated by the following examples, which should not be construed as further limiting. The contents of figures and all references, patents and published patent applications cited throughout this application are expressly incorporated herein by reference.

### V. EXAMPLES

### Example 1: Generation of CD27-Specific Human Monoclonal Antibodies

Human anti-CD27 monoclonal antibodies were generated by immunizing the H2L2 strain of Harbour® transgenic mice with a soluble human CD27 antigen. Harbour® transgenic mice have had the endogenous mouse heavy chain (HC) and kappa light chain (κ-chain) DNA sequences knocked out and have had sequences for the human variable (V) regions and rat constant (C) regions stably incorporated into the mouse genome.

Antigen and Immunization: The antigen was a soluble fusion protein comprising a CD27 extracellular domain with an Fc tag (R&D Systems). The antigen was mixed with MPL plus TDM adjuvant system (Sigma) for immunizations. 5-25 micrograms soluble recombinant CD27 antigen in PBS were mixed 1:1 with the adjuvant. Mice were injected with 200 microliters of the prepared antigen into the peritoneal cavity every 14 days. Animals that developed anti-CD27 titers were given an i.v. injection of 5-10 micrograms soluble recombinant CD27 antigen three to four days prior to fusion. Mouse spleens were harvested, and the isolated splenocytes used for hybridoma preparation.

Hybridoma Preparation: The P3x63Ag8.653 murine myeloma cell line (ATCC CRL 1580) was used for the fusions. RPMI 1640 (Invitrogen) containing 10% FBS was used to culture the myeloma cells. Additional media supplements were added to the Hybridoma growth media, which included: up to 10% Hybridoma Enhancing Supplement (Sigma), 10% FBS (Sigma), L-glutamine (Gibco), 0.1% gentamycin (Gibco), 2-mercaptoethanol (Gibco), with HAT (Sigma; 1.0×10⁴ M hypoxanthine, 4.0×10⁻⁷ M aminopterin, 1.6×10⁻⁵ M thymidine media).

Spleen cells were mixed with the P3x63Ag8.653 myeloma cells in a 6:1 ratio and pelleted by centrifugation. Polyethylene glycol was added dropwise with careful mixing to facilitate fusion. Hybridomas were allowed to grow out for one to two weeks until visible colonies become established. Supernatant was harvested and used for initial screening for rat IgG via ELISA using a human soluble CD27 fusion protein and a rat Fc specific detection. IgG positive supernatants were then assayed for CD27 specificity via flow cytometry. The hybridomas were also screened for cross-reactivity with cynomolgus macaque CD27 and all were positive for binding.

Hybridoma cells were expanded and cell pellets were frozen for RNA isolation and sequencing. The V_{H} and V_{L} coding regions of human monoclonal antibodies were identified using RNA from the corresponding hybridomas. RNA was reverse transcribed to cDNA, the V coding regions were amplified by PCR and the PCR products were sequenced, inserted into a human IgG1kappa vector, transiently expressed and purified by protein A column chromatography which led to the isolation of antibodies of particular interest, which were designated as 2B3 and 3C2.

### Example 2: Assays to Determine Human Monoclonal Antibody Binding Characteristics to CD27

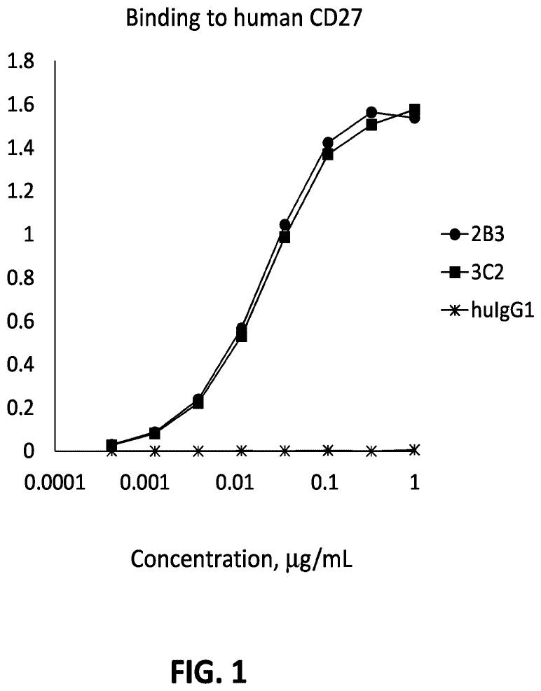

Microtiter plates were coated with recombinant human CD27-FLAG-HIS in PBS, and then blocked with 5% bovine serum albumin in PBS. Protein A purified human monoclonal antibodies were added at various concentrations and incubated at 37° C. The plates were washed with PBS/Tween and then incubated with a goat-anti-human IgG Fc-specific polyclonal reagent conjugated to horseradish peroxidase at 37° C. After washing, the plates were developed with HRP substrate, and analyzed at OD 450-650 nm using a microtiter plate reader. FIG. 1 shows that anti-CD27 antibodies 2B3 and 3C2 bind human CD27.

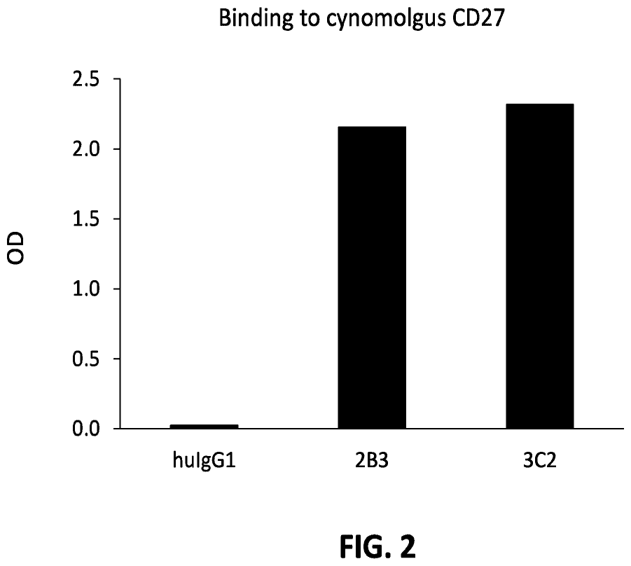

To establish that cynomolgus macaques are a relevant model for testing anti-CD27 monoclonal antibodies, microtiter plates were coated with recombinant cynomolgus CD27-FLAG-HIS in PBS, and then blocked with 5% bovine serum albumin in PBS. Hybridoma supernatants or rat IgG control were added and incubated at 37° C. The plates were washed with PBS/Tween and then incubated with a mouse-anti-rat IgG Fc-specific polyclonal reagent conjugated to horseradish peroxidase at 37° C. After washing, the plates were developed with HRP substrate, and analyzed at OD 450-650 nm using a microtiter plate reader. FIG. 2 shows that anti-CD27 antibodies 2B3 and 3C2 bind cynomolgus CD27.

### Example 3: Binding to CD27 Cells

The ability of anti-CD27 human monoclonal antibodies to bind to CD27 on cells expressing human CD27 on their surface was investigated by flow cytometry as follows: Antibodies were tested for binding to a human cell line expressing human CD27 on their surface. Protein A purified human monoclonal antibodies (3 μg/ml) were incubated with Ramos cells expressing human CD27 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions.

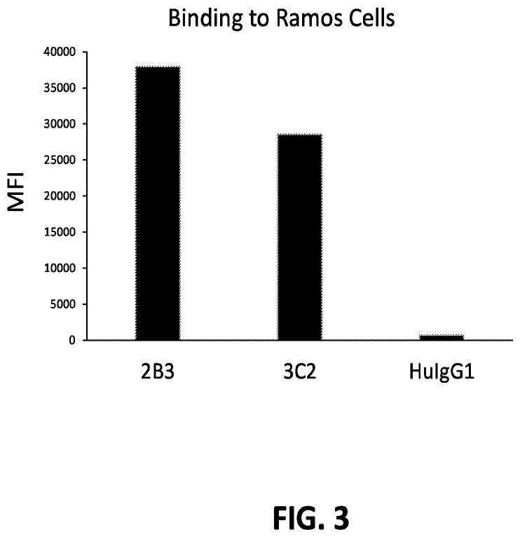

As shown in FIG. 3, the anti-CD27 human monoclonal antibodies demonstrated high levels of binding to cells expressing human CD27.

### Example 4: Binding to Human T Cells

The ability of anti-CD27 human monoclonal antibodies to bind to CD27 on human T cells was investigated by flow cytometry as follows:

Antibodies were tested for binding to human CD3⁺ T cells which express human CD27 on their surface. Human peripheral blood mononuclear cells were isolated from buffy coats using Ficoll separation, and CD3⁺ cells were further isolated from the PBMC's using magnetic bead separation technology from Miltenyi Biotec. Protein A purified human monoclonal antibodies (3 μg/ml) were incubated with the T cells at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions.

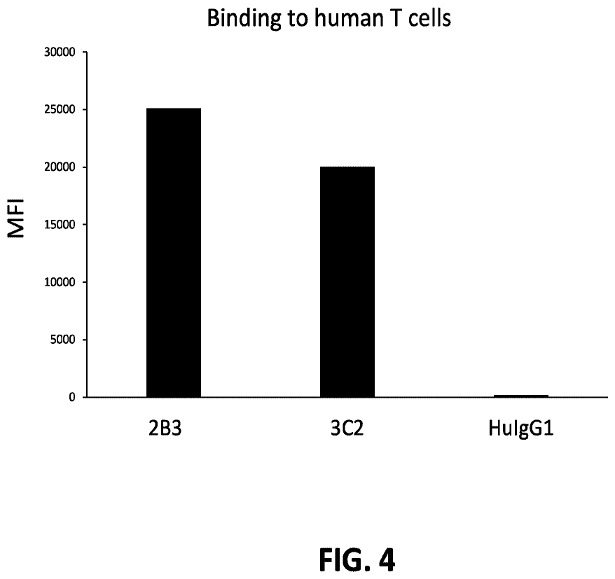

As shown in FIG. 4, the anti-CD27 human monoclonal antibodies demonstrated high levels of binding to human T cells.

### Example 5: Blocking of CD70 Binding

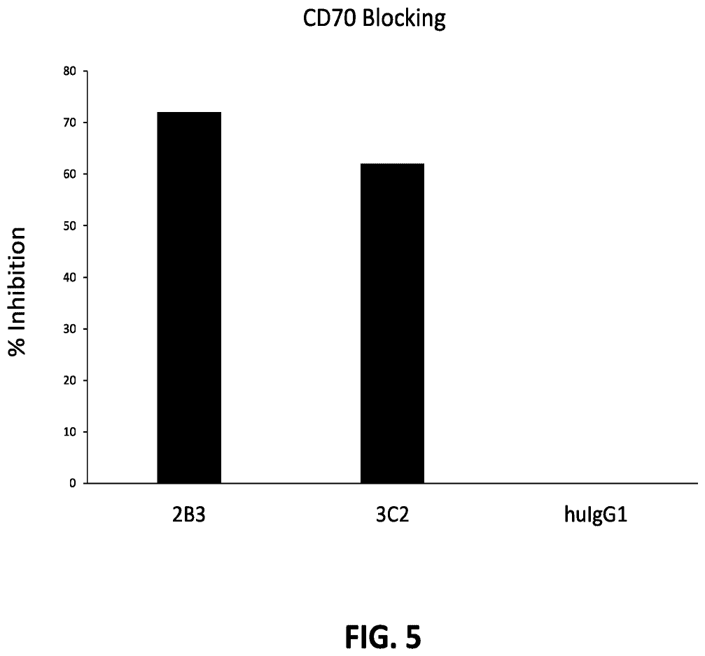

The effect of the human monoclonal antibodies on the binding of soluble CD70 to CD27 on the cell surface was measured by flow cytometry. Ramos cells expressing CD27 were incubated for 5 minutes at room temperature with the antibodies (50 μg/ml), followed by the addition of human CD70-biotin ([final]=0.5 μg/mL) for 20 minutes at room temperature on a plate shaker. CD27 captured CD70 was detected with streptavidin PE and analyzed on a FACSCanto II™ instrument (BD Biosciences, NJ, USA). FIG. 5 shows that anti-CD27 antibodies 2B3 and 3C2 block CD70 binding to CD27 on cells.

### Example 6: NFκB Activation

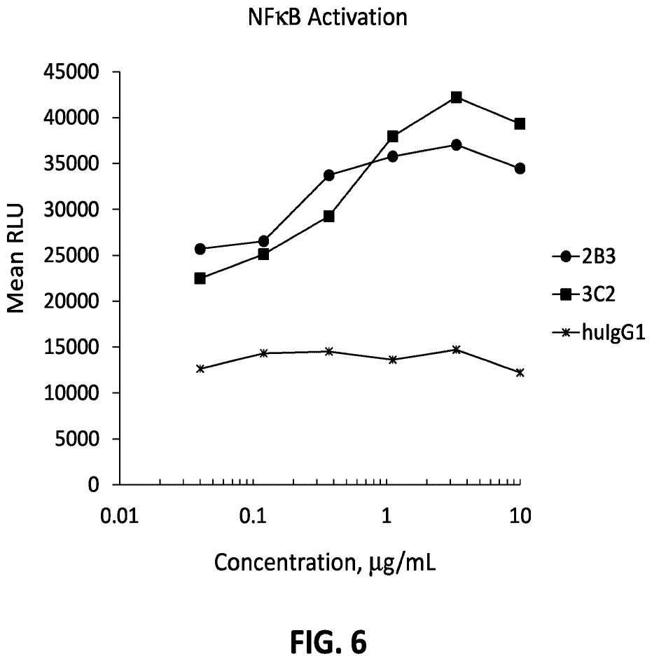

A luciferase reporter cell line expressing CD27 was incubated for 6 hours at 37° C., 6% CO₂ with various concentrations of the human anti-CD27 antibodies. Luciferase is expressed upon activation and was detected with the Luciferase Assay System by Promega according to the manufacturer's guidelines. FIG. 6 shows the high level of NFκB activation induced by 2B3 and 3C2 antibodies as a function of antibody concentration.

### Example 7: T Cell Proliferation

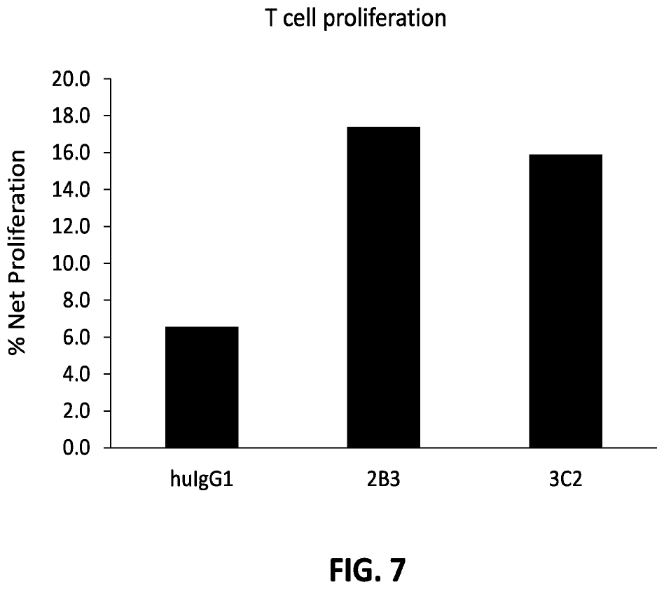

Human Peripheral Blood Mononuclear Cells (PBMCs) isolated from buffy coat preparations and CD3⁺ cells were further isolated from the PBMCs using magnetic bead separation technology from Miltenyi Biotec. The T cells were labeled with 1 mM carboxyfluorescein succinimidyl ester (CFSE) at room temperature while rotating for 5 minutes. The CFSE labeled PBMCs (1×10⁶) were dispensed into wells dry coated with anti-CD3 antibody (OKT3, eBioscience) at 1 μg/mL and the anti-CD27 antibodies or human IgG1 control at 10 μg/mL. The plates were incubated at 37° C., 5% CO₂ for 72 hours. The cells were harvested and analyzed by flow cytometry on a FACSCanto II™ instrument (BD Biosciences, NJ, USA) according to the manufacturer's directions. FIG. 7 shows that antibodies 2B3 and 3C2 significantly increase T cell proliferation.

### Example 8: Generation of PD-L1-Specific Human Monoclonal Antibodies

Human anti-PD-L1 monoclonal antibodies were generated by immunizing the H2L2 strain of Harbour® transgenic mice with a soluble human PD-L1 antigen. Harbour® transgenic mice have had the endogenous mouse heavy chain (HC) and kappa light chain (κ-chain) DNA sequences knocked out and have had sequences for the human variable (V) regions and rat constant (C) regions stably incorporated into the mouse genome.

Antigen and Immunization: The antigen was a soluble fusion protein comprising a PD-L1 extracellular domain with a HIS tag (R&D Systems), or a recombinant human PD-L1-msG2a chimeric protein (made in-house). The antigen was mixed with MPL plus TDM adjuvant system (Sigma) for immunizations. 5-25 micrograms soluble recombinant PD-L1 antigen in PBS were mixed 1:1 with the adjuvant. Mice were injected with 200 microliters of the prepared antigen into the peritoneal cavity every 14 days. Animals that developed anti-PD-L1 titers were given an iv injection of 5-10 micrograms soluble recombinant PD-L1 antigen three to four days prior to fusion. Mouse spleens were harvested, and the isolated splenocytes used for hybridoma preparation.

Hybridoma Preparation: The P3x63Ag8.653 murine myeloma cell line (ATCC CRL 1580) was used for the fusions. RPMI 1640 (Invitrogen) containing 10% FBS was used to culture the myeloma cells. Additional media supplements were added to the Hybridoma growth media, which included: up to 10% Hybridoma Enhancing Supplement (Sigma), 10% FBS (Sigma), L-glutamine (Gibco), 0.1% gentamycin (Gibco), 2-mercaptoethanol (Gibco), with HAT (Sigma; 1.0×10⁴ M hypoxanthine, 4.0×10⁻⁷ M aminopterin, 1.6×10⁻⁵ M thymidine media).

Spleen cells were mixed with the P3x63Ag8.653 myeloma cells in a 6:1 ratio and pelleted by centrifugation. Polyethylene glycol was added dropwise with careful mixing to facilitate fusion. Hybridomas were allowed to grow out for one to two weeks until visible colonies become established. Supernatant was harvested and used for initial screening for rat IgG via ELISA using a human soluble PD-L1 fusion protein and a rat Fc specific detection. IgG positive supernatants were then assayed for PD-L1 specificity via flow cytometry. The hybridomas were also screened for cross-reactivity with cynomolgus macaque PD-L1 and all were positive for binding.

Hybridoma cells were expanded and cell pellets were frozen for RNA isolation and sequencing. The V_{H} and V_{L} coding regions of human monoclonal antibodies were identified using RNA from the corresponding hybridomas. RNA was reverse transcribed to cDNA, the V coding regions were amplified by PCR and the PCR products were sequenced, inserted into human IgG1kappa vector, transiently expressed and purified by protein A column chromatography which led to the isolation of a number of antibodies of particular interest, which were designated as 1B3, 3B6, 4A3, 7H7, 8B1 and 9H9.

### Example 9: Assays to Determine Human Monoclonal Antibody Binding Characteristics to PD-L1

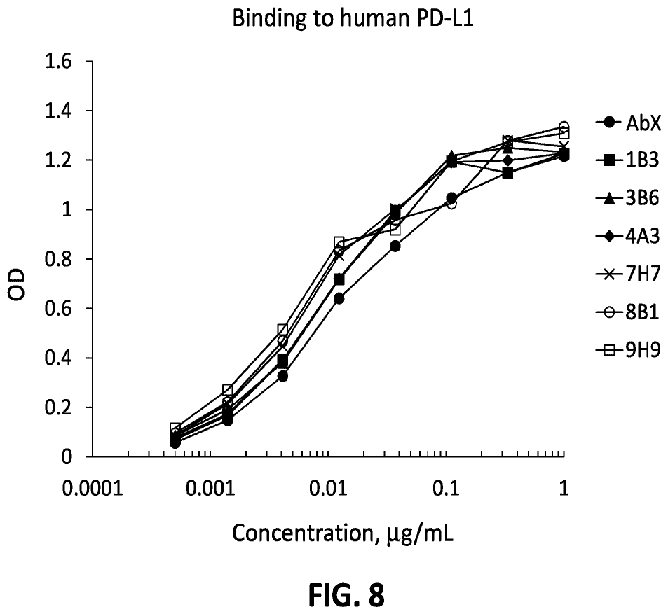

Microtiter plates were coated with recombinant human PD-L1-msFc in PBS, and then blocked with 5% bovine serum albumin in PBS. Protein A purified human monoclonal antibodies were added at various concentrations and incubated at 37° C. The plates were washed with PBS/Tween and then incubated with a goat-anti-human IgG Fc-specific polyclonal reagent conjugated to horseradish peroxidase at 37° C. After washing, the plates were developed with HRP substrate, and analyzed at OD 450-650 nm using a microtiter plate reader. FIG. 8 shows that the anti-PD-L1 antibodies strongly bind human PD-L1 as a function of antibody concentration.

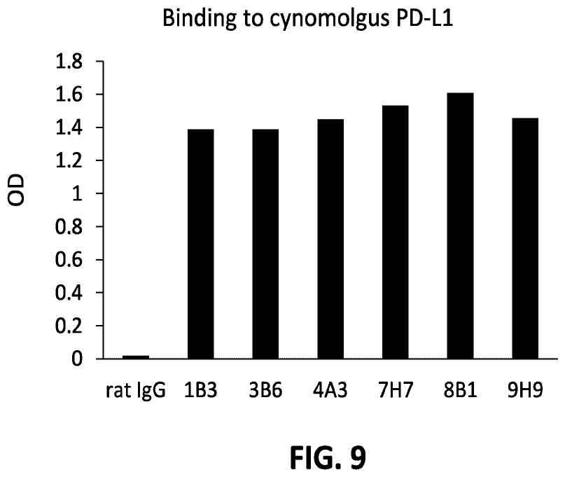

To establish that cynomolgus macaques are a relevant model for testing anti-PD-L1 monoclonal antibodies, microtiter plates were coated with recombinant cynomolgus PD-L1-FLAG-HIS in PBS, and then blocked with 5% bovine serum albumin in PBS. Hybridoma supernatants or rat IgG control were added and incubated at 37° C. The plates were washed with PBS/Tween and then incubated with a mouse-anti-rat IgG Fc-specific polyclonal reagent conjugated to horseradish peroxidase at 37° C. After washing, the plates were developed with HRP substrate, and analyzed at OD 450-650 nm using a microtiter plate reader. FIG. 9 shows that the anti-PD-L1 antibodies bind cynomolgus PD-L1.

### Example 10: Blocking of PD1 Binding

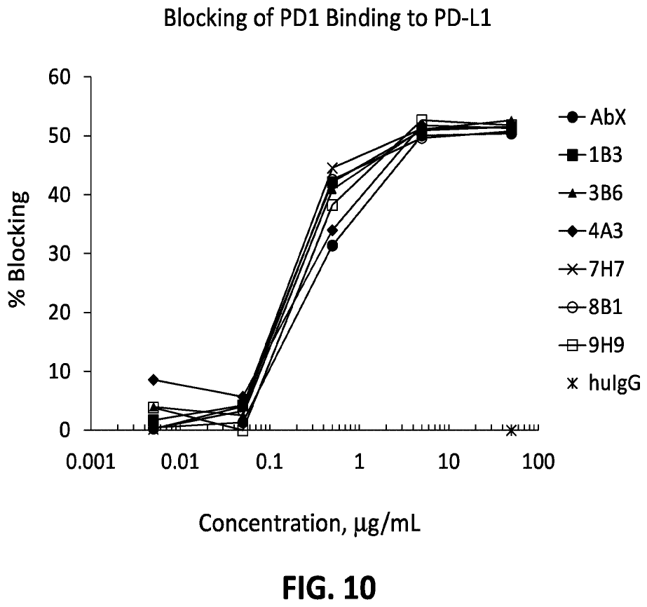

The effect of the human monoclonal antibodies on the binding of soluble PD1 to PD-L1 on the cell surface was measured by flow cytometry. 293 cells expressing PD-L1 were incubated for 5 minutes at room temperature with the antibodies, followed by the addition of human PD1-biotin ([final]=0.5 mg/mL). PD-L1 captured PD1 was detected with streptavidin PE and analyzed on a FACSCanto II™ instrument (BD Biosciences, NJ, USA). FIG. 10 shows that the anti-PD-L1 antibodies block PD-L1 binding to PD1 as a function of antibody concentration.

### Example 11: Binding to PD-L1 Cells

The ability of anti-PD-L1 human monoclonal antibodies to bind to PD-L1 on cells expressing human PD-L1 on their surface was investigated by flow cytometry as follows:

Antibodies were tested for binding to a human cell line expressing human PD-L1 on their surface. Protein A purified human monoclonal antibodies were incubated with 293 cells expressing human PD-L1 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions.

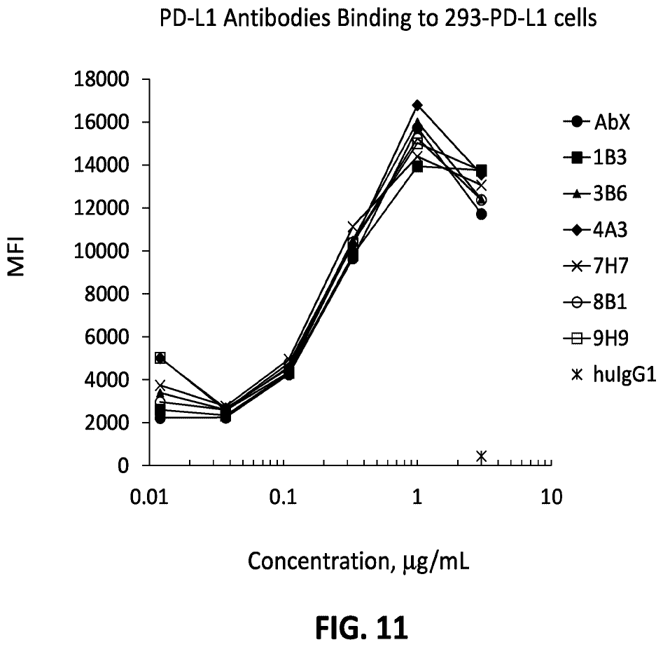

As shown in FIG. 11, the anti-PD-L1 human monoclonal antibodies demonstrated high levels of binding to cells expressing human PD-L1 as a function of antibody concentration.

### Example 12: Binding to Human Dendritic Cells

The ability of anti-PD-L1 human monoclonal antibodies to bind to PD-L1 on human dendritic cells was investigated by flow cytometry as follows:

Antibodies were tested for binding to human dendritic cells which express human PD-L1 on their surface. Dendritic cells were generated as follows: PMBC's were added to a T175 cm² flasks and monocytes allowed to adhere for ˜2 hours at 37° C., 6% CO₂. The non-adherent cells were removed and the monocytes cultured for 7 days in RPMI containing 10% FBS, 10 ng/mL IL-4 (R&D Systems) and 100 ng/mL GM-CSF (R&D Systems). Non-adherent cells were harvested and confirmed to be dendritic cells by expression of CD11c (not shown). Protein A purified human monoclonal antibodies were incubated with the dendritic cells at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions.

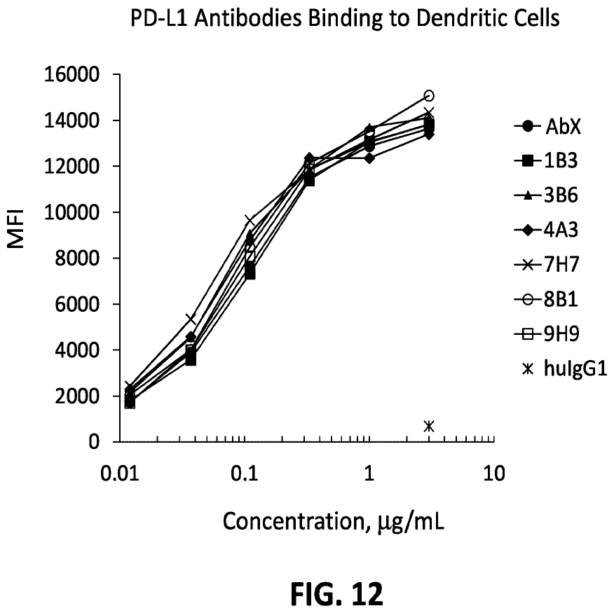

As shown in FIG. 12, the anti-PD-L1 human monoclonal antibodies demonstrated high levels of binding to human dendritic cells as a function of antibody concentration.

### Example 13: T Cell PD1/PD-L1 Blockade Bioassay

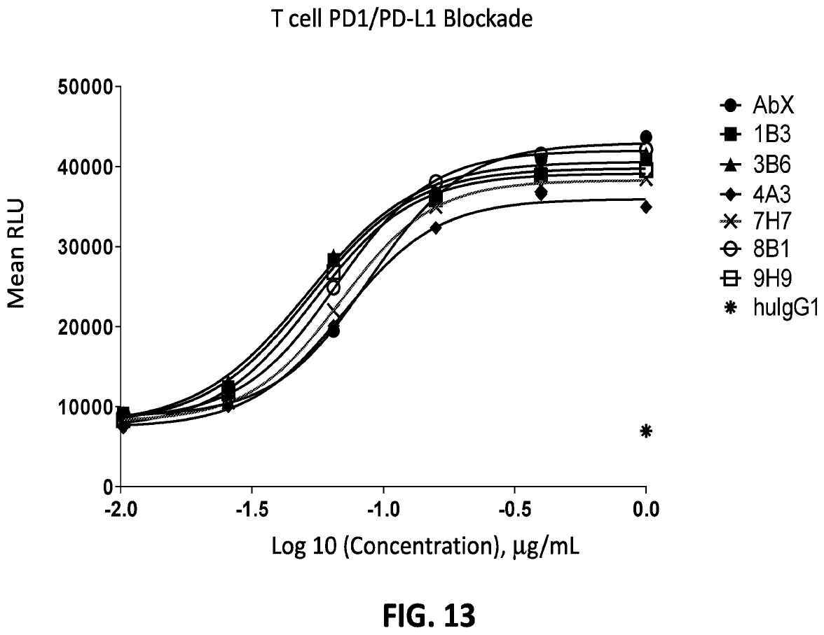

The effect of the PD-L1 antibodies on blockade of the PD1/PD-L1 interaction was determined using the PD1/PD-L1 Blockade Assay from Promega. Two engineered cell lines, PD1 Effector cells and PD-L1 aAPC/CHO-K1 cells were co-cultured in the presence of the antibodies for 6 hours. Blocking of the PD1/PD-L1 interaction results in TCR activation and induces luminescence via the NFAT pathway. Luminescence was detected by the addition of Bio-Glo reagent and quantitated on a Perkin Elmer Victor X luminometer. As shown on FIG. 13, the anti-PD-L1 antibodies effectively block the PD1/PD-L1 interaction between cells leading to activation of the NFAT pathway.

### Example 14: Mixed Lymphocyte Reaction

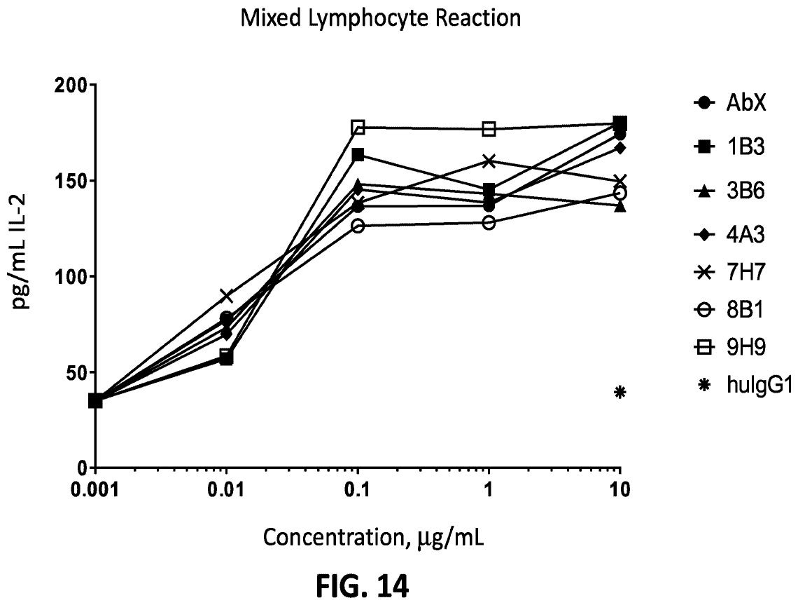

Human peripheral blood mononuclear cells were isolated from buffy coats using Ficoll separation, and CD4⁺ cells were further isolated from the PBMC's using magnetic bead separation technology from Miltenyi Biotec. Allogeneic dendritic cells were generated as follows: PMBC's were added to a T175 cm² flasks and monocytes allowed to adhere for ˜2 hours at 37° C., 6% CO₂. The non-adherent cells were removed and the monocytes cultured for 7 days in RPMI containing 10% FBS, 10 ng/ml IL-4 (R&D Systems) and 100 ng/ml GM-CSF (R&D Systems). Non-adherent cells were harvested and confirmed to be dendritic cells by expression of CD11c (not shown). The CD4+ cells and DC's were co-incubated at a 10:1 ratio in the presence of the antibody dilutions for 3 days. Supernatants were harvested and analyzed for IL-2 production by ELISA (R&D Systems). As shown on FIG. 14, the anti-PD-L1 antibodies were able to induce a significant mixed lymphocyte response.

### Example 15: Development and Functional Testing of Bispecific Constructs

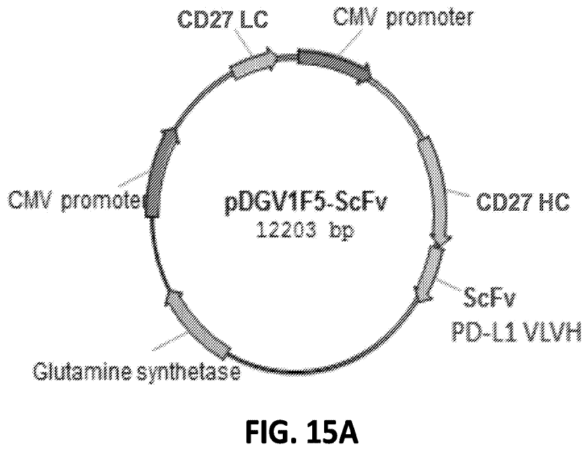

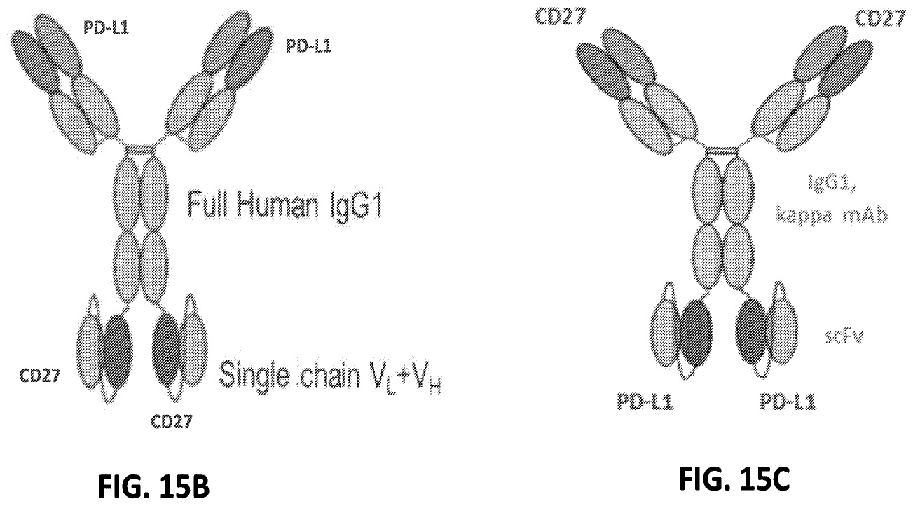

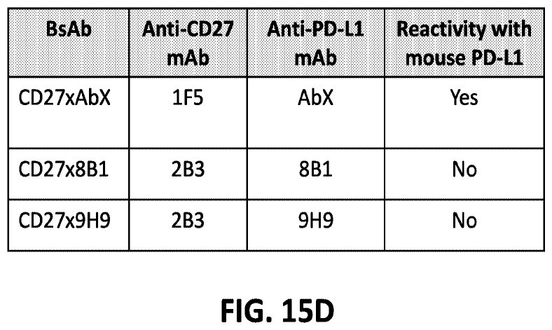

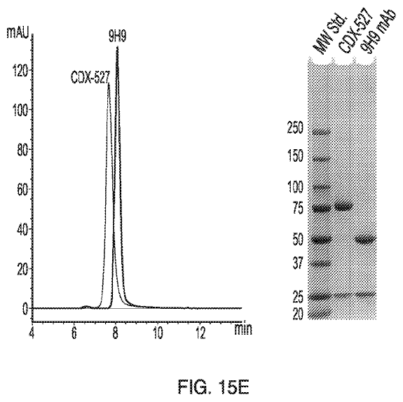

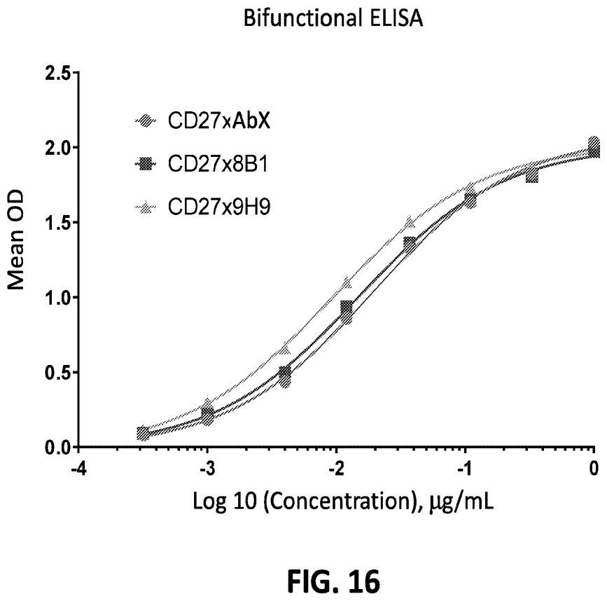

Tetravalent bispecific constructs were developed using a fully human IgG1 backbone for the PD-L1 monoclonal antibody and the scFv of the CD27 monoclonal antibody genetically linked to the c-terminus of the heavy chain. Alternative bispecific constructs also were developed using a fully human IgG1 backbone for the CD27 monoclonal antibody and the scFv of the PD-L1 monoclonal antibody. FIG. 15A shows a representative vector containing the CD27 light chain, CD27 heavy chain, and the c-terminal PD-L1 single chain Fv (VL+VH) polypeptide. FIGS. 15B and 15C are two alternative depictions of the CD27/PD-L1 bispecific format. FIG. 15B shows the CD27/PD-L1 bispecific antibody protein wherein an anti-PD-L1 antibody is linked to an anti-CD27 scFv (referred to herein as “CDX-527” when the anti-PD-L1 antibody is 9H9 and the anti-CD27 scFv is derived from 2B3. Otherwise AbXx2B3) and FIG. 15C shows the CD27/PD-L1 bispecific antibody protein wherein an anti-CD27 antibody is linked to an anti-PD-L1 scFv. FIG. 15D is a table of representative anti-CD27/anti-PD-L1 bispecific constructs generated.

The full 9H9x2B3 (CDX-527) heavy chain sequence was as follows (with the IgG1 constant region sequence shown in bold):

(SEQ ID NO: 179)
EVQLVESGGGLVQPGGSLRLSCAASGGIISTYWMSWVRQAPGKGLEWVANIKQDGS
EKYYVDSVKGRFTISRDNAKNSLYLQMNSLRVEDTAMYYCARDRPVAGASALWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHN
HYTQKSLSLSPGKGSSGGGGSEIVMTQSPATLSVSPGERATLSCRASQSIRSNLAWY
QQKPGQAPRLLIYGASTRATGIPARFSGSGSGTEFTLTISSLQSENFAVYYCQQYNNW
PLTFGCGTKVEIKGGGGSGGGGSGGGGSGGGGSQVQLVQSGAEVKKPGASVKVSC
KASGYTFTGYYIHWVRQAPGQCLEWMGWINPNSGGTNSAQKFQDRVTITRVTSINT
AYMELSRLRSDDTAVYFCARDRLVLPWFGEIFPDAFDIWGQGTLVTVSS

The 9H9x2B3 (CDX-527) light chain sequence was as follows (with the constant region sequence shown in bold):

(SEQ ID NO: 180)
DIQMTQSPSTLSASVGDRVTITCRASQSISGWLAWYQQKPGKAPKLLIY
KASSLESGVPSRFSGSGSGTEFTLTISSLQPDDFATYYCQQYYGSSRTF
GQGTNVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

### Example 16: Assays to Determine Bispecific Monoclonal Antibody Binding Characteristics and Functional Activity

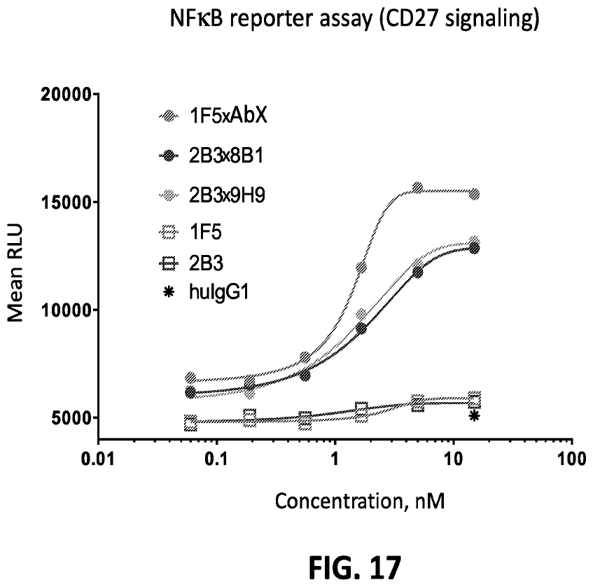

Binding of bispecific constructs to CD27 and PD-L1 was assessed using a bifunctional ELISA. Antibody AbX is a known anti-PD-L1 monoclonal antibody. In brief, a microtiter plate was coated with human CD27-FLAG-HIS. Dilutions of the bispecific constructs were allowed to bind before adding human PD-L1-msFc that was detected with an HRP labeled goat anti-mouse IgG (Fc specific) antibody. Representative binding curves for three bispecific constructs (CD27xAbX, CD27x8B1, and CD27x9H9) are shown in FIG. 16. All three antibodies demonstrated significant binding to CD27 and PD-L1.

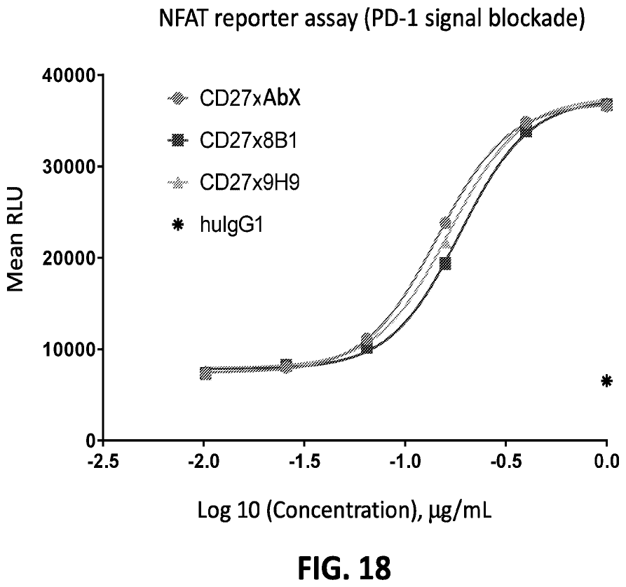

CD27 pathway activation was assessed by measuring NFκB activation. In brief, CD27 was transfected into a NFκB-luciferase reporter cell line (Signosis). The cells were incubated for 6 hours with each bispecific construct or antibody (1F5xAbX, 2B3x8B1, 2B3x9H9, 1F5, 2B3, or huIgG1) and luciferase expression was detected with the Brite-Glo™ system (Promega). Note: the reporter cell line is positive for human PD-L1. FIG. 17 shows the level of NFκB activation induced by the antibodies as a function of concentration. Additionally, the bispecific constructs 1F5xAbX, 2B3x8B1, and 2B3x9H9 showed significantly higher NFκB activation than 1F5 or 2B3 alone.

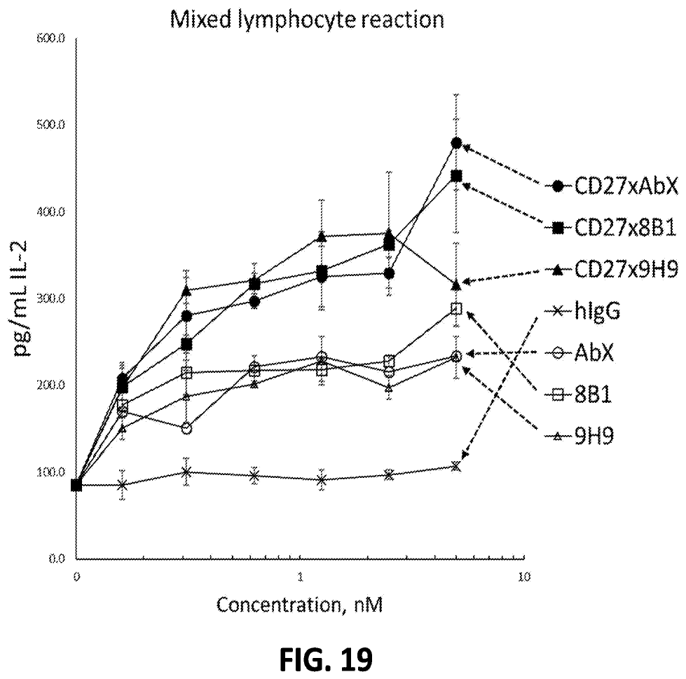

PD-1 signaling blockade was assessed by measuring NFAT pathway activation. In brief, PD-1 Effector cells and PD-L1 aAPC cells were co-cultured in the presence of dilutions of each bispecific construct or the control antibody (CD27xAbX, CD27x8B1, CD27x9H9, or huIgG1). Activation of the NFAT pathway via PD-L1/PD-1 blockade is detected by addition of Bio-Glo™ reagent. (Commercially available kit from Promega). As shown in FIG. 18, the bispecific constructs induced strong NFAT pathway activation as a function of antibody concentration.

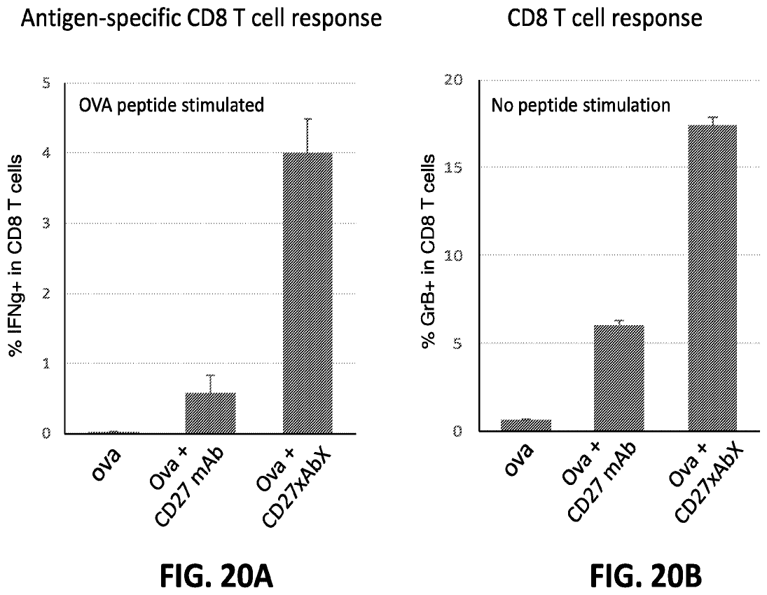

IL-2 production/secretion was also measured in a mixed lymphocyte reaction. In brief, CD4 cells were incubated in the presence of allogeneic dendritic cells and dilutions of each bispecific constructs or antibody (CD27xAbX, CD27x8B1, CD27x9H9, huIgG1, AbX, 8B1, or 9H9) for 3 days. Supernatants were harvest and IL-2 levels were assessed by ELISA (R&D Systems). Representative IL-2 concentration curves are shown in FIG. 19. The bispecific constructs CD27xAbX, CD27x8B1 and CD27x9H9 showed significantly higher IL-2 production/secretion than AbX (a known anti-PD-L1 monoclonal antibody), 8B1, or 9H9 alone (e.g., about 2× higher IL-2 production).

### Example 17: Assays to Determine In Vivo Activity of CD27/PD-L1 Bispecific Constructs

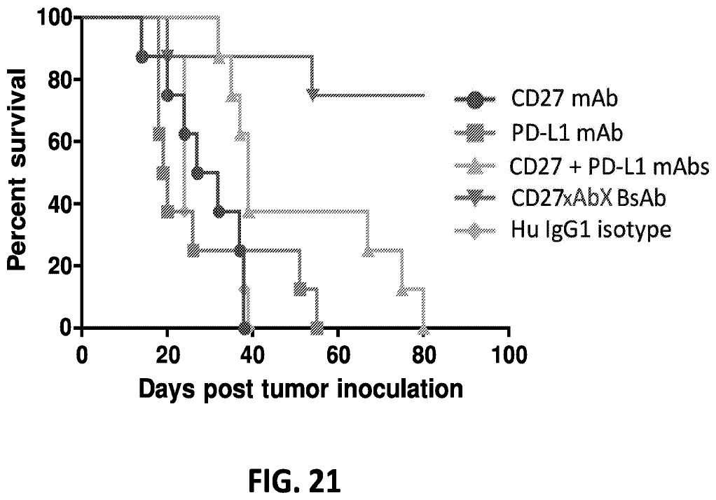

HuCD27-Tg mice were injected with 0.1 mg of bispecific CD27xAbX construct (BsAb) or CD27 monospecific antibody (mAb), and 5 mg of Ovalbumin on day 0, as indicated in the x-axis of each graph. On Day 7 spleen cells were harvested and intracellular cytokine IFNγ and IL2, and cytolysis enzyme granzyme B (GrB) were detected by flow cytometry analysis with and without ex-vivo stimulation of SIINFEKL peptide (SEQ ID NO: 188). Percentage of SIINFEKL (SEQ ID NO: 188)-specific IFN-γ⁺ and IL2⁺ in CD8 T cells (FIG. 20A), and percentage of GrB+ in CD8 T cells without SIINFEKL (SEQ ID NO: 188) stimulation (FIG. 20B) are shown, indicating the CD27xAbX bispecific construct induces a significant CD8 T cell response as compared to CD27 monoclonal antibody alone (e.g., about 2.5× to about 8× higher response).

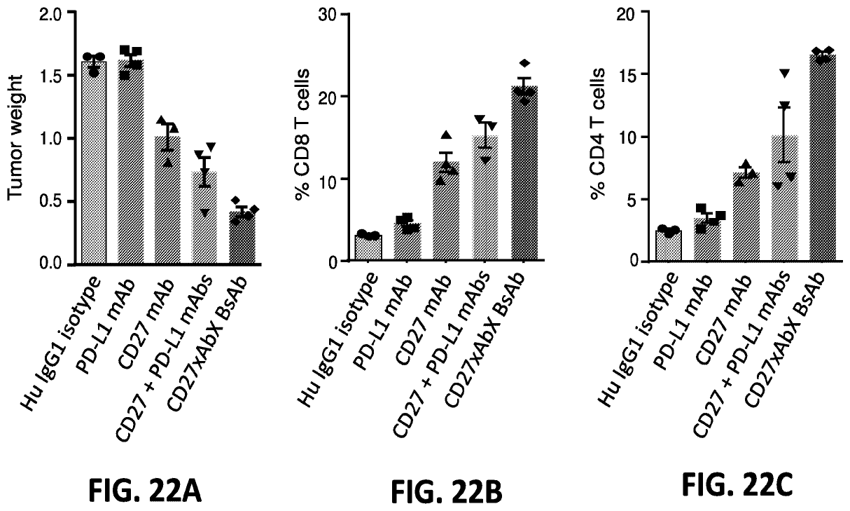

Tumor growth, survival, and tumor infiltrate numbers were also measured in mice treated with the BsAb or mAbs. HuCD27-Tg mice were injected i.v. with BCL1 cells (5×10⁶) on day 0. Antibodies or the bispecific construct were injected i.p (0.2 mg) on day 5. Mice were divided into two groups. One group was used to measure survival (n=8). FIG. 21 shows percent survival over time of mice treated with either CD27 monoclonal antibody, PD-L1 monoclonal antibody, combination of CD27+PD-L1 monoclonal antibodies, and CD27xAbX BsAb. Mice treated with CD27+PD-L1 antibodies survived significantly longer than mice treated with either CD27 monoclonal antibody, PD-L1 monoclonal antibody, or Hu IgG1 alone (e.g., 1.5×-2× longer). Moreover, mice treated with the CD27xAbX BsAb survived significantly longer than mice treated with any of the other antibodies tested either alone or in combination. In fact, 70% to 80% of mice treated with CD27xAbX BsAb were still alive after 80 days post tumor inoculation whereas mice in other groups had all died. Three surviving mice were re-challenged with same number of BCL1 cells after 180 days follow-up, and they were protected from the re-challenge.

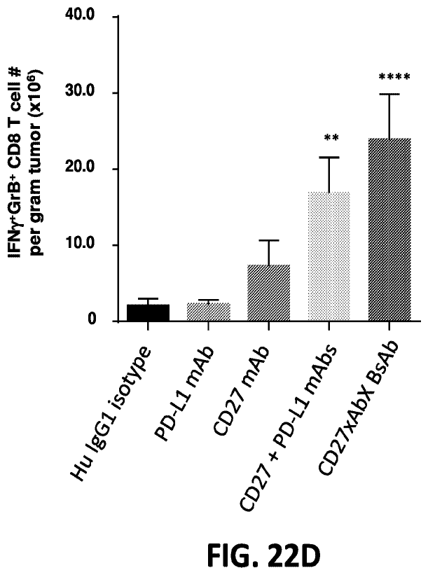

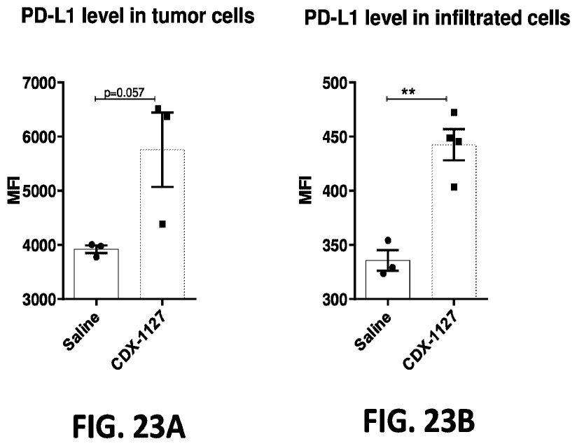

The second group of mice was used to measure tumor weight and T cell levels on day 11. FIGS. 22A-22D show tumor weight, percent CD8 T cells, percent CD4 T cells, and IFNγ and GrB double positive CD8 T cells, respectively. Similar to the survival data, CD27xAbX BsAb significantly reduced tumor weight (e.g., 1.5× to 3× less), and significantly increased T cell numbers and activity (e.g., 1.5× to 4× more), as compared to any of the other antibodies tested either alone or in combination. FIGS. 23A and 23B show the upregulation of PD-L1 expression level on the surface of BCL1 lymphoma cells (FIG. 23A) and tumor microenvironment infiltrated cells (FIG. 23B) upon CD27 mAb treatment (CDX-1127), providing rationale for the combination or BsAb of anti-CD27 and anti-PD-L1.

### Example 18: Bifunctional ELISA

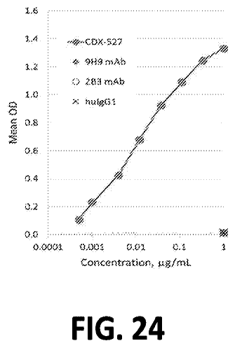

Characterization and binding of the bispecific construct CDX-527 (prepared as in Example 15) was analyzed. FIG. 15E shows characterization of the bispecific antibody CDX-527 by HPLC and gel electrophoresis (reducing conditions). Binding to both CD27 and PD-L1 was determined using a bifunctional ELISA generally as described in Example 16. Results are shown in FIG. 24 from which it can be seen that CDX-527 demonstrated significant binding to CD27 and PD-L1.

### Example 19: Activation of NFκB by Bispecific Construct CDX-527

Activation of NFκB by the bispecific construct CDX-527 (prepared as in Example 15) was determined generally as described in Example 16 except that SteadyGlo™ reagent from Promega was used instead of BriteGlo™.

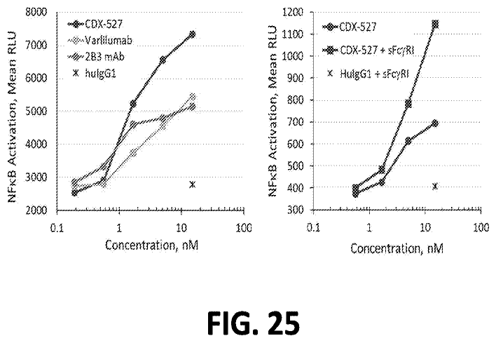

Results are shown in FIG. 25, which shows the higher level of activation by CDX-527 compared to the monospecific anti-CD27 antibodies 1F5 and 2B3 alone. Activation was also measured in the presence of soluble FcγR1, which further increased NFκB activation as shown.

### Example 20: Mixed Lymphocyte Reaction

The ability of the bispecific construct CDX-527 (prepared as in Example 15) to induce a mixed lymphocyte response was determined by the method described generally in Example 14, with testing for IL-2.

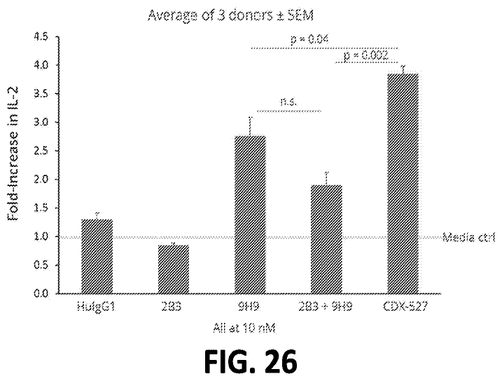

Results are shown in FIG. 26, from which it can be seen that CDX-527 was able to induce a significant mixed lymphocyte response which was also significantly greater than that of the monospecific antibodies 2B3 and 9H9 when used either alone or in combination.

### Example 21: T-Cell Activation

Human Peripheral Blood Mononuclear Cells (PBMCs) were isolated from buffy coat preparations and CD3+ cells were further isolated from the PBMCs using magnetic bead separation technology from Miltenyi Biotec. CD3+ cells (1×10⁵) were dispensed into wells coated with anti-CD3 antibody (OKT3, eBioscience) and soluble human PD-L1. Antibodies 2B3 and 9H9 or CDX-527 (prepared as in Example 15) were added to the cells at concentrations between 0.1 nM and 10 nM. The plates were incubated at 37° C., 5% CO₂ for 72 hours at which time IL-2 levels in the supernatants were measured.

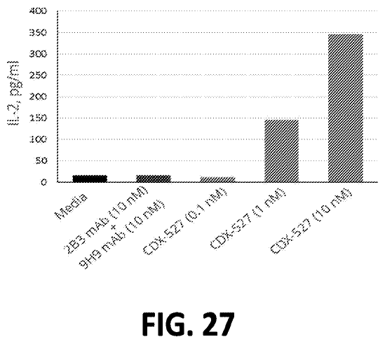

Results are shown in FIG. 27 from which it can be seen that CDX-527 significantly activated T-cells and did so to a significantly greater extent than the monospecific antibodies 2B3 and 9H9 when used in combination.

### Example 22: Pharmacokinetics

Pharmacokinetics of CDX-527 was studied in non-human primates (NHPs) at a dose level of 7.0 mg/kg and volume of 3.0 ml/kg i.v. No significant change was observed in any clinical parameters during the 21 day study. Serum levels of CDX-527 were determined by ELISA.

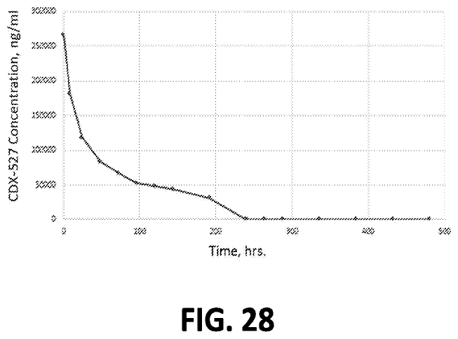

Results are shown in FIG. 28. Pharmacokinetic analysis resulted in T½ of approximately 110 hours.

### Example 23: Cell Based PD1/PDL1 Blockade Assay

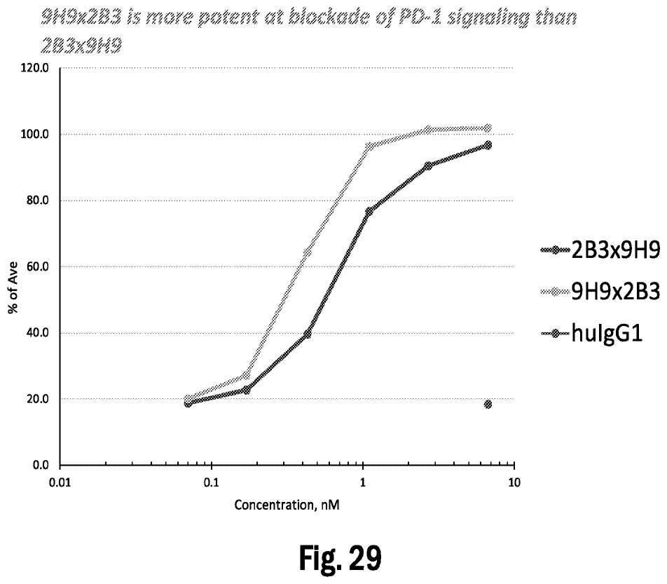

Bispecific antibodies 9H9x2B3 and the opposite configuration 2B3x9H9 were compared in a cell based PD1/PDL1 blockade assay generally as described in Example 13. Results are shown in FIG. 29 from which it can be seen that the 9H9x2B3 configuration was more potent at blockade of PD-1 signaling than the 2B3x9H9 configuration.

### Example 24: Mixed Lymphocyte Reaction

Bispecific antibodies 9H9x2B3 and the opposite configuration 2B3x9H9 were compared in a mixed lymphocyte reaction generally as described in Example 14.

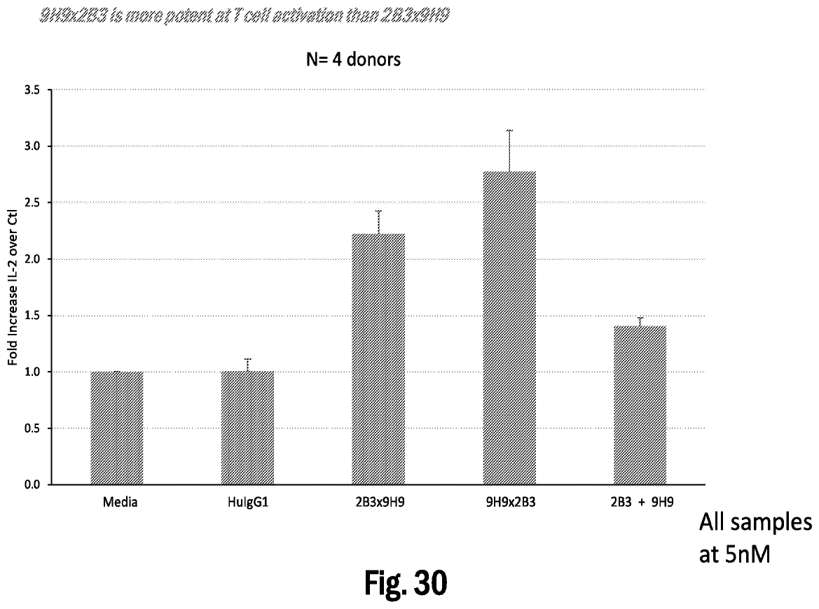

Results are shown in FIG. 30 from which it can be seen that the 9H9x2B3 configuration was more potent at T-cell activation than the 2B3x9H9 configuration.

### Example 25: Vaccine Induced CD8 T Cell Response

Bispecific antibodies AbXx2B3 and the opposite configuration 2B3xAbX were compared in a vaccine model of T-cell response generally as described in Example 17.

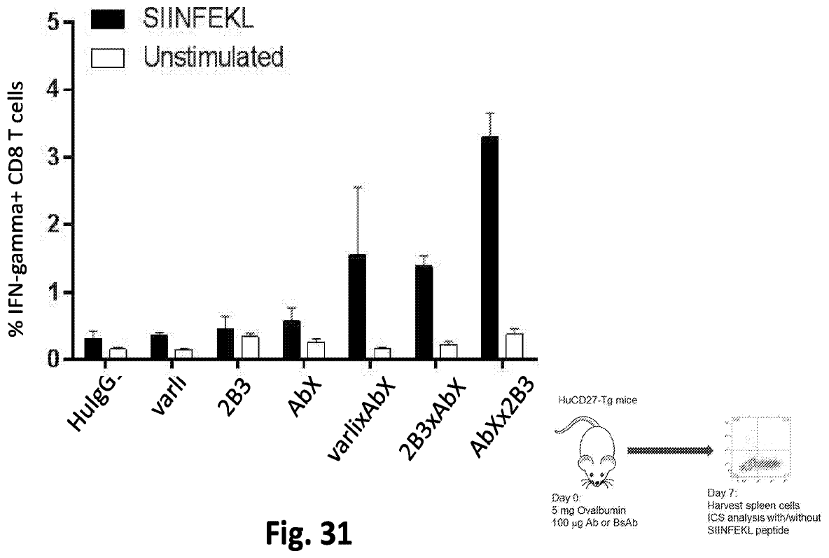

Results are shown in FIG. 31 from which it can be seen that AbXx2B3 was more potent than 2B3xAbX in stimulating a vaccine induced CD8+ T-cell response.

### Example 26: BCL1 Tumor Model

Bispecific antibodies AbXx2B3 and the opposite configuration 2B3xAbX were compared in a BCL1 Tumor Model generally as described in Example 17.

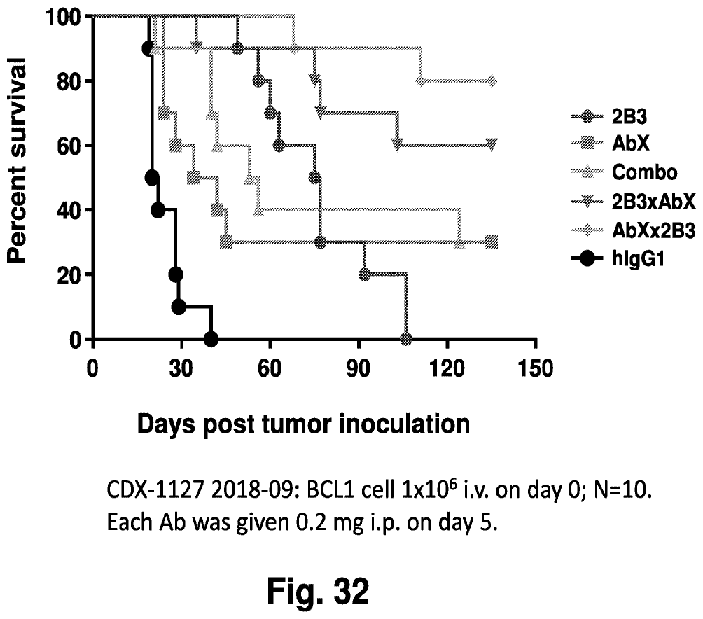

Results are shown in FIG. 32 from which it can be seen that AbXx2B3 had greater anti-tumor activity than 2B3xAbX.

### Example 27: Blocking of PD-L1 Binding to CD80

A microtiter plate was coated with recombinant human CD80, then blocked. Biotinylated huPD-L1 was pre-incubated for 1 hour at room temperature with 50 μg/mL of the huIgG1 control or the anti-PD-L1 antibodies (AbX or 9H9), then added to the plate. Streptavidin-HRP was used to detect the binding of the PD-L1 to CD80.

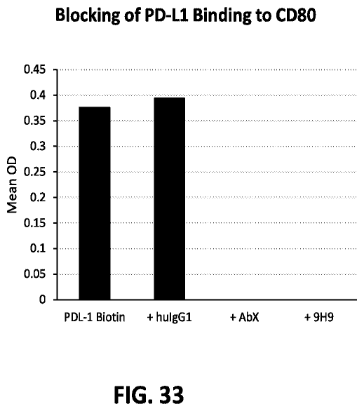

Results are shown in FIG. 33 from which it can be seen that the anti-PD-L1 antibodies completely blocked PD-L1 binding to CD80.

### Example 28: 2B3 Binding to huCD27

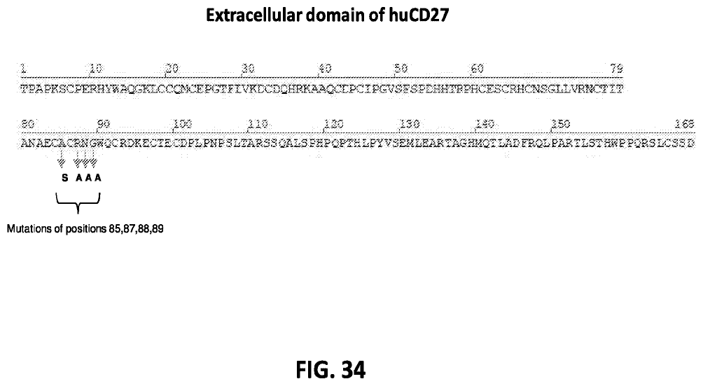

The full length wild type extracellular domain (ECD) of human CD27 or human CD27 with mutated amino acids 85, 87, 88, and 89 (A85S, R87A, N88A and G89A; see FIG. 34) were coated to a plate, then blocked. Supernatant from transiently transfected cells expressing mAb 2B3 was added and binding was detected with an HRP conjugated goat anti-human IgG Fc polyclonal antibody.

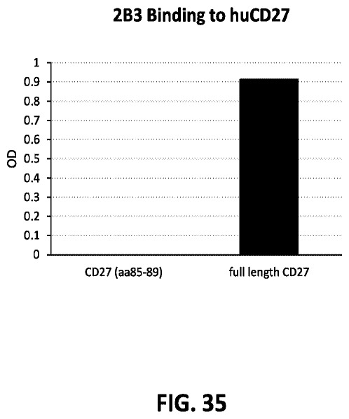

Results are shown in FIG. 35 from which it can be seen that the anti-CD27 antibody 2B3 did not bind to the mutated CD27 ECD. Accordingly, this indicates that antibody 2B3 binds to an epitope on human CD27 involving or including one or more residues within amino acids 80-95, e.g., one or more residues within amino acids 85-89, of the extracellular domain (ECD) of human CD27 (SEQ ID NO: 183), e.g., amino acids 85, 87, 88, and/or 89 of the ECD of human CD27 (SEQ ID NO: 183).

### Example 29: Increasing 9H9x2B3 Expression Through Protein Engineering: 9H9-2B3 (DD)

To increase expression of the bispecific product the rare valine (V) residue at H72 in FR3 of the 2B3 heavy chain and the rare asparagine (N) residue at L82 in FR3 of the 2B3 light chain were each changed to aspartic acid (D) residues (ie V72D and N82D respectively).

The modified 2B3 heavy chain sequence was as follows (the CDRs are underlined and the modified residue in FR3 is double underlined):

(SEQ ID NO: 177)
QVQLVQSGAEVKKPGASVKVSCKASGYTFTGYYIHWVRQAPGQGLEWMG
WINPNSGGTNSAQKFQDRVTITRDTSINTAYMELSRLRSDDTAVYFCAR
DRLVLPWFGEIFPDAFDIWGQGTLVTVSS

The modified 2B3 light chain sequence was as follows (the CDRs are underlined and the modified residue in FR3 is double underlined):

(SEQ ID NO: 178)
EIVMTQSPATLSVSPGERATLSCRASQSIRSNLAWYQQKPGQAPRLLIY
GASTRATGIPARFSGSGSGTEFTLTISSLQSEDFAVYYCQQYNNWPLTF
GGGTKVEIK

These sequences (designated 2B3 (DD)) were used in an scFv format which was genetically linked to the C-terminus of the heavy chains of the 9H9 antibody. The resulting 9H9x2B3 (DD) bispecific antibody was designated 9H9x2B3 (DD).

The full 9H9x2B3 (DD) heavy chain sequence was as follows (with the IgG1 constant “backbone” sequence shown in bold):

(SEQ ID NO: 181)
EVQLVESGGGLVQPGGSLRLSCAASGGIISTYWMSWVRQAPGKGLEWVANIKQDGS
EKYYVDSVKGRFTISRDNAKNSLYLQMNSLRVEDTAMYYCARDRPVAGASALWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHN
HYTQKSLSLSPGKGSSGGGGSEIVMTQSPATLSVSPGERATLSCRASQSIRSNLAWY
QQKPGQAPRLLIYGASTRATGIPARFSGSGSGTEFTLTISSLQSEDFAVYYCQQYNNW
PLTFGCGTKVEIKGGGGSGGGGSGGGGSGGGGSQVQLVQSGAEVKKPGASVKVSC
KASGYTFTGYYIHWVRQAPGQCLEWMGWINPNSGGTNSAQKFQDRVTITRDTSINT
AYMELSRLRSDDTAVYFCARDRLVLPWFGEIFPDAFDIWGQGTLVTVSS

The 9H9x2B3 (DD) light chain sequence was as follows (with the constant region shown in bold):

(SEQ ID NO: 182)
DIQMTQSPSTLSASVGDRVTITCRASQSISGWLAWYQQKPGKAPKLLIY
KASSLESGVPSRFSGSGSGTEFTLTISSLQPDDFATYYCQQYYGSSRTF
GQGTNVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The DNA sequence of the 9H9x2B3 (DD) variable domain sequence was as follows:

(SEQ ID NO: 185)
GAAGTGCAACTGGTGGAGTCGGGTGGTGGACTCGTGCAGCCCGGCGGAT
CCCTGAGACTCTCTTGTGCCGCATCGGGCGGCATTATTAGCACTTACTG
GATGTCATGGGTCAGACAGGCACCGGGAAAGGGCTTGGAATGGGTGGCG
AATATCAAGCAGGATGGATCCGAGAAGTACTACGTGGACTCCGTGAAGG
GCAGATTCACCATTTCCCGGGACAACGCCAAGAACTCGCTCTATCTGCA
AATGAACTCGTTGCGGGTGGAAGATACTGCCATGTACTACTGCGCCCGG
GACCGGCCTGTGGCCGGGGCGTCGGCCCTCTGGGGCCAGGGCACTCTGG
TCACCGTGTCCTCT

The DNA sequence of the 9H9x2B3 (DD) scFv domain sequence was as follows (connector and linker sequences are shown in bold):

(SEQ ID NO: 186)
GGCTCCAGCGGGGGTGGCGGTTCCGAGATCGTGATGACTCAGAGCCCGG
CAACCCTGTCCGTGTCTCCGGGGGAGCGGGCTACTCTTTCCTGCCGGGC
ATCCCAGTCCATCCGGTCGAACCTTGCGTGGTACCAACAGAAGCCTGGA
CAGGCGCCCCGCCTGCTGATCTACGGGGCGTCGACTAGGGCCACCGGCA
TCCCGGCCCGCTTCTCCGGGTCCGGATCCGGCACCGAATTCACCCTCAC
CATCTCGAGCCTGCAGTCCGAAAACTTCGCCGTCTACTACTGCCAGCAG
TACAACAACTGGCCGCTGACATTCGGATGCGGAACCAAAGTGGAAATCA
AGGGCGGCGGCGGATCCGGCGGTGGCGGCAGCGGCGGTGGAGGATCCGG
TGGCGGCGGTTCACAAGTGCAGCTGGTGCAGTCAGGCGCCGAAGTCAAG
AAGCCCGGGGCCAGCGTGAAAGTCAGCTGCAAGGCTTCCGGATACACCT
TCACGGGTTACTACATTCACTGGGTTCGCCAAGCGCCCGGGCAGTGTCT
GGAGTGGATGGGATGGATCAACCCTAACTCGGGGGGAACCAACTCGGCC
CAAAAGTTCCAGGACCGGGTCACCATTACAAGAGTCACGTCCATCAACA
CTGCCTACATGGAGTTGAGCCGGCTGCGATCAGACGACACCGCCGTGTA
CTTCTGCGCGAGGGACCGCCTCGTCCTCCCGTGGTTTGGAGAGATCTTC
CCGGATGCCTTCGACATTTGGGGACAGGGGACCCTCGTGACTGTGTCCA
GC

The DNA sequence of the 9H9x2B3 (DD) light chain variable sequence was as follows:

(SEQ ID NO: 187)
GATATCCAGATGACCCAGAGCCCGTCCACCCTTTCCGCGAGCGTCGGCG
ACAGAGTGACCATTACTTGTCGGGCCTCGCAAAGCATCTCCGGCTGGCT
GGCTTGGTACCAGCAAAAGCCTGGAAAGGCCCCTAAGCTGCTGATCTAC
AAGGCCTCATCCCTGGAGTCCGGAGTGCCTTCACGCTTTTCGGGGAGCG
GATCGGGGACTGAGTTCACCCTCACCATTTCCTCCCTGCAACCCGACGA
TTTCGCGACATACTACTGCCAGCAGTACTACGGTTCCTCGCGCACGTTC
GGACAGGGCACTAACGTCGAGATCAAG

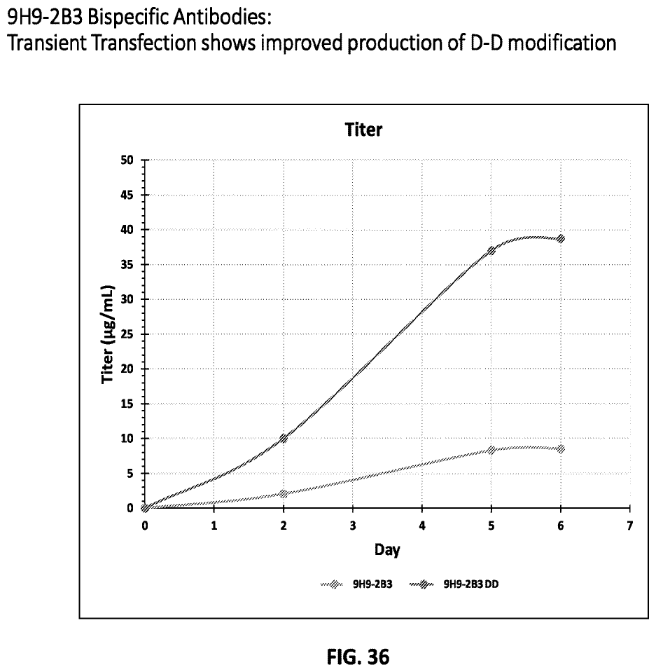

As can be seen from FIG. 36 transient transfections showed improved production (expression) of the 9H9-2B3 (DD) modified construct compared to the original (unmodified) 9H9-2B3 construct.

SUMMARY OF SEQUENCE LISTING

| | |
|---|---|
| SEQ ID NO: 1 | GYYWS |
| 3C2 V_{H} CDR1 | |
| SEQ ID NO: 2 | YNYYSGSTNYNPSLKS |
| 3C2 V_{H} CDR2 | |
| SEQ ID NO: 3 | YPLIRGAFDY |
| 3C2 V_{H} CDR3 | |
| SEQ ID NO: 4 | RSSQNLLHTNGYNYLD |
| 3C2 V_{L} CDR1 | |
| SEQ ID NO: 5 | LGSNRAS |
| 3C2 V_{L} CDR2 | |
| SEQ ID NO: 6 | MQALQTPLT |
| 3C2 V_{L} CDR3 | |
| SEQ ID NO: 7 | GYYIH |
| 2B3 V_{H} CDR1 | |
| SEQ ID NO: 8 | WINPNSGGTNSAQKFQD |
| 2B3 V_{H} CDR2 | |
| SEQ ID NO: 9 | DRLVLPWFGEIFPDAFDI |
| 2B3 V_{H} CDR3 | |
| SEQ ID NO: 10 | RASQSIRSNLA |
| 2B3 V_{L} CDR1 | |
| SEQ ID NO: 11 | GASTRAT |
| 2B3 V_{L} CDR2 | |
| SEQ ID NO: 12 | QQYNNWPLT |
| 2B3 V_{L} CDR3 | |
| SEQ ID NO: 13 | MKHLWFCLLLVAAPRWVLSQAQLQESGPGLVKPSETLSLTCTVSTGSIS |
| 3C2 V_{H}-with | GYYWSWIRQPPGKGLEWIGYNYYSGSTNYNPSLKSRVTISIDTSKNQFS |
| signal sequence | LKLNSVTAADTAVYYCARYPLIRGAFDYWGQGTLVTVSS |
| SEQ ID NO: 14 | MRLPAQLLGLLMLWVSGSSGDIVMTQSPLSLPVTPGEPASISCRSSQNL |
| 3C2 V_{L}-with | LHTNGYNYLDWYLQKPGQSPQLLIYLGSNRASGVPDRFSGSGSGTDFT |
| signal sequence | LKISRVEAEDVGVYYCMQALQTPLTFGGGTKVEIK |
| SEQ ID NO: 15 | MDWTWRILFLVAAATGAHSQVQLVQSGAEVKKPGASVKVSCKASGY |
| 2B3 V_{H}-with | TFTGYYIHWVRQAPGQGLEWMGWINPNSGGTNSAQKFQDRVTITRVT |
| signal sequence | SINTAYMELSRLRSDDTAVYFCARDRLVLPWFGEIFPDAFDIWGQGTLV |
| | TVSS |
| SEQ ID NO: 16 | MEAPAQLLFLLLLWLPDSTGEIVMTQSPATLSVSPGERATLSCRASQSIR |
| 2B3 V_{L}-with | SNLAWYQQKPGQAPRLLIYGASTRATGIPARFSGSGSGTEFTLTISSLQS |
| signal sequence | ENFAVYYCQQYNNWPLTFGGGTKVEIK |
| SEQ ID NO: 17 | QAQLQESGPGLVKPSETLSLTCTVSTGSISGYYWSWIRQPPGKGLEWIG |
| 3C2 V_{H}- | YNYYSGSTNYNPSLKSRVTISIDTSKNQFSLKLNSVTAADTAVYYCARY |
| without signal | PLIRGAFDYWGQGTLVTVSS |
| sequence | |
| SEQ ID NO: 18 | DIVMTQSPLSLPVTPGEPASISCRSSQNLLHTNGYNYLDWYLQKPGQSP |
| 3C2 V_{L}- | QLLIYLGSNRASGVPDRFSGSGSGTDFTLKISRVEAEDVGVYYCMQAL |
| without signal | QTPLTFGGGTKVEIK |
| sequence | |
| SEQ ID NO: 19 | QVQLVQSGAEVKKPGASVKVSCKASGYTFTGYYIHWVRQAPGQGLE |
| 2B3 V_{H}- | WMGWINPNSGGTNSAQKFQDRVTITRVTSINTAYMELSRLRSDDTAVY |
| without signal | FCARDRLVLPWFGEIFPDAFDIWGQGTLVTVSS |
| sequence | |
| SEQ ID NO: 20 | EIVMTQSPATLSVSPGERATLSCRASQSIRSNLAWYQQKPGQAPRLLIY |
| 2B3 V_{L}- | GASTRATGIPARFSGSGSGTEFTLTISSLQSENFAVYYCQQYNNWPLTFG |
| without signal | GGTKVEIK |
| sequence | |
| SEQ ID NO: 21 | ATGAAACATCTGTGGTTCTGCCTTCTCCTGGTGGCAGCTCCCAGATG |
| 3C2 V_{H} DNA | GGTCCTGTCCCAGGCGCAGCTGCAGGAGTCGGGCCCAGGACTGGTG |
| Sequence-with | AAGCCTTCGGAGACCCTGTCCCTCACCTGCACTGTCTCTACTGGCTC |
| signal sequence | CATCAGTGGTTACTACTGGAGCTGGATCCGGCAGCCCCCAGGGAAG |
| | GGACTGGAGTGGATTGGGTATAATTATTACAGTGGGAGCACCAACT |
| | ACAACCCCTCCCTCAAGAGTCGAGTCACCATATCAATAGACACGTC |
| | CAAGAACCAGTTCTCCCTGAAGCTGAATTCTGTGACCGCTGCGGAC |
| | ACGGCCGTATATTACTGTGCGAGATATCCTCTGATTCGGGGAGCTTT |
| | TGACTACTGGGGCCAGGGAACCCTGGTCACCGTCTCCTCA |
| SEQ ID NO: 22 | ATGAGGCTCCCTGCTCAGCTCCTGGGGCTGCTAATGCTCTGGGTCTC |
| 3C2 V_{L} DNA | TGGATCCAGTGGGGATATTGTGATGACTCAGTCTCCACTCTCCCTGC |
| Sequence-with | CCGTCACCCCTGGAGAGCCGGCCTCCATCTCCTGTAGGTCTAGTCAG |
| signal sequence | AACCTCCTGCATACTAATGGCTACAACTATTTGGATTGGTACCTGCA |
| | GAAGCCAGGGCAGTCTCCACAGCTCCTGATCTATTTGGGTTCTAATC |
| | GGGCCTCCGGGGTCCCTGACAGGTTCAGTGGCAGTGGATCAGGCAC |
| | AGATTTTACACTGAAAATCAGCAGAGTGGAGGCTGAGGATGTTGGG |
| | GTTTATTACTGCATGCAAGCTCTACAAACTCCGCTCACTTTCGGCGG |
| | AGGGACCAAGGTGGAGATCAAA |
| SEQ ID NO: 23 | ATGGACTGGACCTGGAGGATCCTCTTCTTGGTGGCAGCAGCCACAG |
| 2B3 V_{H} DNA | GAGCCCACTCCCAGGTGCAGCTGGTGCAGTCTGGGGCTGAGGTGAA |
| Sequence-with | GAAGCCTGGGGCCTCAGTGAAGGTCTCCTGCAAGGCTTCTGGATAC |
| signal sequence | ACCTTCACCGGCTACTATATACACTGGGTGCGACAGGCCCCTGGAC |
| | AAGGGCTTGAGTGGATGGGATGGATCAACCCTAACAGTGGTGGCAC |
| | AAACTCTGCACAGAAGTTTCAGGACAGGGTCACCATCACCAGGGTC |
| | ACGTCCATCAACACAGCCTACATGGAGCTGAGCAGACTGAGATCTG |
| | ACGACACGGCCGTGTATTTCTGTGCGAGAGATCGGCTCGTATTACCA |
| | TGGTTCGGGGAAATATTCCCAGATGCTTTTGATATCTGGGGCCAAGG |
| | GACATTGGTCACCGTCTCTTCA |
| SEQ ID NO: 24 | ATGGAAGCCCCAGCGCAGCTTCTCTTCCTCCTGCTACTCTGGCTCCC |
| 2B3 V_{L} DNA | AGATTCCACTGGAGAAATAGTGATGACGCAGTCTCCAGCCACCCTG |
| Sequence-with | TCTGTGTCTCCAGGGGAAAGAGCCACCCTCTCCTGCAGGGCCAGTC |
| signal sequence | AGAGTATTAGGAGCAACTTAGCCTGGTATCAGCAGAAACCTGGCCA |
| | GGCTCCCAGGCTCCTCATCTATGGTGCATCCACCAGGGCCACTGGTA |
| | TCCCAGCCAGGTTCAGTGGCAGTGGGTCTGGGACAGAGTTCACTCTC |
| | ACCATCAGCAGCCTGCAGTCTGAAAATTTTGCAGTTTATTACTGTCA |
| | GCAGTATAATAACTGGCCTCTCACTTTCGGCGGAGGGACCAAGGTG |
| | GAGATCAAA |
| SEQ ID NO: 25 | CAGGCGCAGCTGCAGGAGTCGGGCCCAGGACTGGTGAAGCCTTCGG |
| 3C2 V_{H} DNA | AGACCCTGTCCCTCACCTGCACTGTCTCTACTGGCTCCATCAGTGGT |
| Sequence- | TACTACTGGAGCTGGATCCGGCAGCCCCCAGGGAAGGGACTGGAGT |
| without signal | GGATTGGGTATAATTATTACAGTGGGAGCACCAACTACAACCCCTC |
| sequence | CCTCAAGAGTCGAGTCACCATATCAATAGACACGTCCAAGAACCAG |
| | TTCTCCCTGAAGCTGAATTCTGTGACCGCTGCGGACACGGCCGTATA |
| | TTACTGTGCGAGATATCCTCTGATTCGGGGAGCTTTTGACTACTGGG |
| | GCCAGGGAACCCTGGTCACCGTCTCCTCA |
| SEQ ID NO: 26 | GATATTGTGATGACTCAGTCTCCACTCTCCCTGCCCGTCACCCCTGG |
| 3C2 V_{L} DNA | AGAGCCGGCCTCCATCTCCTGTAGGTCTAGTCAGAACCTCCTGCATA |
| Sequence- | CTAATGGCTACAACTATTTGGATTGGTACCTGCAGAAGCCAGGGCA |
| without signal | GTCTCCACAGCTCCTGATCTATTTGGGTTCTAATCGGGCCTCCGGGG |
| sequence | TCCCTGACAGGTTCAGTGGCAGTGGATCAGGCACAGATTTTACACTG |
| | AAAATCAGCAGAGTGGAGGCTGAGGATGTTGGGGTTTATTACTGCA |
| | TGCAAGCTCTACAAACTCCGCTCACTTTCGGCGGAGGGACCAAGGT |
| | GGAGATCAAA |
| SEQ ID NO: 27 | CAGGTGCAGCTGGTGCAGTCTGGGGCTGAGGTGAAGAAGCCTGGGG |
| 2B3 V_{H} DNA | CCTCAGTGAAGGTCTCCTGCAAGGCTTCTGGATACACCTTCACCGGC |
| Sequence- | TACTATATACACTGGGTGCGACAGGCCCCTGGACAAGGGCTTGAGT |
| without signal | GGATGGGATGGATCAACCCTAACAGTGGTGGCACAAACTCTGCACA |
| sequence | GAAGTTTCAGGACAGGGTCACCATCACCAGGGTCACGTCCATCAAC |
| | ACAGCCTACATGGAGCTGAGCAGACTGAGATCTGACGACACGGCCG |
| | TGTATTTCTGTGCGAGAGATCGGCTCGTATTACCATGGTTCGGGGAA |
| | ATATTCCCAGATGCTTTTGATATCTGGGGCCAAGGGACATTGGTCAC |
| | CGTCTCTTCA |
| SEQ ID NO: 28 | GAAATAGTGATGACGCAGTCTCCAGCCACCCTGTCTGTGTCTCCAGG |
| 2B3 V_{L} DNA | GGAAAGAGCCACCCTCTCCTGCAGGGCCAGTCAGAGTATTAGGAGC |
| Sequence- | AACTTAGCCTGGTATCAGCAGAAACCTGGCCAGGCTCCCAGGCTCC |
| without signal | TCATCTATGGTGCATCCACCAGGGCCACTGGTATCCCAGCCAGGTTC |
| sequence | AGTGGCAGTGGGTCTGGGACAGAGTTCACTCTCACCATCAGCAGCC |
| | TGCAGTCTGAAAATTTTGCAGTTTATTACTGTCAGCAGTATAATAAC |
| | TGGCCTCTCACTTTCGGCGGAGGGACCAAGGTGGAGATCAAA |
| SEQ ID NO: 29 | TSWMS |
| 7H7 V_{H} CDR1 | |
| SEQ ID NO: 30 | NIKQDGSEKYYVDSVKG |
| 7H7 V_{H} CDR2 | |
| SEQ ID NO: 31 | DRPVAGASAL |
| 7H7 V_{H} CDR3 | |
| SEQ ID NO: 32 | RASQSISGWLA |
| 7H7 V_{L} CDR1 | |
| SEQ ID NO: 33 | KASSLES |
| 7H7 V_{L} CDR2 | |
| SEQ ID NO: 34 | QQYYGSSRT |
| 7H7 V_{L} CDR3 | |
| SEQ ID NO: 35 | TSWMS |
| 1B3 V_{H} CDR1 | |
| SEQ ID NO: 36 | NIKQDGSEKYYVDSVKG |
| 1B3 V_{H} CDR2 | |
| SEQ ID NO: 37 | DRPVAGASAL |
| 1B3 V_{H} CDR3 | |
| SEQ ID NO: 38 | RASQSISGWLA |
| 1B3 V_{L} CDR1 | |
| SEQ ID NO: 39 | KASSLES |
| 1B3 V_{L} CDR2 | |
| SEQ ID NO: 40 | QQYYGSSRT |
| 1B3 V_{L} CDR3 | |
| SEQ ID NO: 41 | TYWMS |
| 3B6 V_{H} CDR1 | |
| SEQ ID NO: 42 | NIKQDGSEKYYVDSVKG |
| 3B6 V_{H} CDR2 | |
| SEQ ID NO: 43 | DRPVAGASAL |
| 3B6 V_{H} CDR3 | |
| SEQ ID NO: 44 | RASQSISGWLA |
| 3B6 V_{L} CDR1 | |
| SEQ ID NO: 45 | KASSLES |
| 3B6 V_{L} CDR2 | |
| SEQ ID NO: 46 | QQYYGSSRT |
| 3B6 V_{L} CDR3 | |
| SEQ ID NO: 47 | THWMS |
| 8B1 V_{H} CDR1 | |
| SEQ ID NO: 48 | NIKQDGSEKYYVDSVKG |
| 8B1 V_{H} CDR2 | |
| SEQ ID NO: 49 | DRPVAGASAL |
| 8B1 V_{H} CDR3 | |
| SEQ ID NO: 50 | RASQSISGWLA |
| 8B1 V_{L} CDR1 | |
| SEQ ID NO: 51 | KASSLES |
| 8B1 V_{L} CDR2 | |
| SEQ ID NO: 52 | QQYYGSSRT |
| 8B1 V_{L} CDR3 | |
| SEQ ID NO: 53 | SSWMS |
| 4A3 V_{H} CDR1 | |
| SEQ ID NO: 54 | NIKQDGSEKYYVDSVKG |
| 4A3 V_{H} CDR2 | |
| SEQ ID NO: 55 | DRPVAGASAL |
| 4A3 V_{H} CDR3 | |
| SEQ ID NO: 56 | RASQSISGWLA |
| 4A3 V_{L} CDR1 | |
| SEQ ID NO: 57 | KASSLES |
| 4A3 V_{L} CDR2 | |
| SEQ ID NO: 58 | QQYYGSSRT |
| 4A3 V_{L} CDR3 | |
| SEQ ID NO: 59 | TYWMS |
| 9H9 V_{H} CDR1 | |
| SEQ ID NO: 60 | NIKQDGSEKYYVDSVKG |
| 9H9 V_{H} CDR2 | |
| SEQ ID NO: 61 | DRPVAGASAL |
| 9H9 V_{H} CDR3 | |
| SEQ ID NO: 62 | RASQSISGWLA |
| 9H9 V_{L} CDR1 | |
| SEQ ID NO: 63 | KASSLES |
| 9H9 V_{L} CDR2 | |
| SEQ ID NO: 64 | QQYYGSSRT |
| 9H9 V_{L} CDR3 | |
| SEQ ID NO: 65 | MELGLSXVFLVAILEGVQCEVQLVESGGGLVQPGGSLRLSCAASGGTIS |
| 7H7 V_{H}-with | TSWMSWVRQAPGKGLEWVANIKQDGSEKYYVDSVKGRFTISRDNAK |
| signal sequence | NSLYLQMNSLRVEDTAIYYCARDRPVAGASALWGQGTLVTVSS |
| SEQ ID NO: 66 | MRVPAQLLGLLLLWLPGAKCDIQMTQSPSTLSASVGDRVTITCRASQSI |
| 7H7 V_{L}-with | SGWLAWYQQKQGKAPKLLIYKASSLESGVPSRFSGSGSGTEFTLTISSL |
| signal sequence | QPDDFATYYCQQYYGSSRTFGQGTNVEIK |
| SEQ ID NO: 67 | MELGLSWVFLVAILEGVQCEVQLVESGGGLVQPGGSLRLSCAASGGTI |
| 1B3 V_{H}-with | STSWMSWVRQAPGKGLEWVANIKQDGSEKYYVDSVKGRFTISRDNAK |
| signal sequence | NSLYLQMNSLRVEDTAMYYCARDRPVAGASALWGQGTLVTVSS |
| SEQ ID NO: 68 | MRVPAQLLGLLLLWLPGAKCDIQMTQSPSTLSASVGDRVTITCRASQSI |
| 1B3 V_{L}-with | SGWLAWYQQKPGKAPKLLIYKASSLESGVPSRFSGSGSGTEFTLTISSLQ |
| signal sequence | PDDFATYYCQQYYGSSRTFGQGTNVEIK |
| SEQ ID NO: 69 | MELGLSWVFLVAILEGVQCEVQLVESGGGLVQPGGSLRLSCAASGGTT |
| 3B6 V_{H}-with | STYWMSWVRQAPGKGLEWVANIKQDGSEKYYVDSVKGRFTISRDNA |
| signal sequence | KNSLNLQMNSLRVEDTAIYYCARDRPVAGASALWGQGTLVTVSS |
| SEQ ID NO: 70 | MRVPAQLLGLLLLWLPGAKCDIQMTQSPSTLSASVGDRVTITCRASQSI |
| 3B6 V_{L}-with | SGWLAWYQQKPGKAPKLLIYKASSLESGVPSRFSGSGSGTEFTLTISSLQ |
| signal sequence | PDDFATYYCQQYYGSSRTFGQGTNVEIK |
| SEQ ID NO: 71 | MELGLSWVFLVAILEGVKCEVRLVESGGGLVQPGGSLRLSCAASGDIIS |
| 8B1 V_{H}-with | THWMSWVRQAPGKGLEWVANIKQDGSEKYYVDSVKGRFTISRDNAK |
| signal sequence | NSLYLQMNTLRVEDTAIYYCTRDRPVAGASALWGQGTLVTVSS |
| SEQ ID NO: 72 | MRVPAQLLGLLLLWLPGAKCDIQMTQSPSTLSASVGDRVTITCRASQSI |
| 8B1 V_{L}-with | SGWLAWYQQKPGKAPKLLIYKASSLESGVPLRFSGSGSGTEFTLTISSL |
| signal sequence | QPDDFATYYCQQYYGSSRTFGQGTNVEIK |
| SEQ ID NO: 73 | MELGLSWVFLVAILEGVQCEVQLVESGGGLVQPGGSLRLSCAASGGIIS |
| 4A3 V_{H}-with | SSWMSWVRQAPGKGLEWVANIKQDGSEKYYVDSVKGRFTISRDNAK |
| signal sequence | DLLYLQMNSLRVEDTALYYCARDRPVAGASALWGQGTLVTVSS |
| SEQ ID NO: 74 | MRVPAQLLGLLLLWLPGAKCDIQMTQSPSTLSASVGDRVTITCRASQSI |
| 4A3 V_{L}-with | SGWLAWYQQKPGKAPKLLIYKASSLESGVPSRFSGSGSGTEFTLTISSLQ |
| signal sequence | PDDFATYYCQQYYGSSRTFGQGTNVEIK |
| SEQ ID NO: 75 | MELGLSWVFLVAILEGVQCEVQLVESGGGLVQPGGSLRLSCAASGGIIS |
| 9H9 V_{H}-with | TYWMSWVRQAPGKGLEWVANIKQDGSEKYYVDSVKGRFTISRDNAK |
| signal sequence | NSLYLQMNSLRVEDTAMYYCARDRPVAGASALWGQGTLVTVSS |
| SEQ ID NO: 76 | MRVPAQLLGLLLLWLPGAKCDIQMTQSPSTLSASVGDRVTITCRASQSI |
| 9H9 V_{L}-with | SGWLAWYQQKPGKAPKLLIYKASSLESGVPSRFSGSGSGTEFTLTISSLQ |
| signal sequence | PDDFATYYCQQYYGSSRTFGQGTNVEIK |
| SEQ ID NO: 77 | EVQLVESGGGLVQPGGSLRLSCAASGGTISTSWMSWVRQAPGKGLEW |
| 7H7 V_{H}- | VANIKQDGSEKYYVDSVKGRFTISRDNAKNSLYLQMNSLRVEDTAIYY |
| without signal | CARDRPVAGASALWGQGTLVTVSS |
| sequence | |
| SEQ ID NO: 78 | DIQMTQSPSTLSASVGDRVTITCRASQSISGWLAWYQQKQGKAPKLLIY |
| 7H7 V_{L}- | KASSLESGVPSRFSGSGSGTEFTLTISSLQPDDFATYYCQQYYGSSRTFG |
| without signal | QGTNVEIK |
| sequence | |
| SEQ ID NO: 79 | EVQLVESGGGLVQPGGSLRLSCAASGGTISTSWMSWVRQAPGKGLEW |
| 1B3 V_{H}- | VANIKQDGSEKYYVDSVKGRFTISRDNAKNSLYLQMNSLRVEDTAMY |
| without signal | YCARDRPVAGASALWGQGTLVTVSS |
| sequence | |
| SEQ ID NO: 80 | DIQMTQSPSTLSASVGDRVTITCRASQSISGWLAWYQQKPGKAPKLLIY |
| 1B3 V_{L}- | KASSLESGVPSRFSGSGSGTEFTLTISSLQPDDFATYYCQQYYGSSRTFG |
| without signal | QGTNVEIK |
| sequence | |
| SEQ ID NO: 81 | EVQLVESGGGLVQPGGSLRLSCAASGGTTSTYWMSWVRQAPGKGLE |
| 3B6 V_{H}- | WVANIKQDGSEKYYVDSVKGRFTISRDNAKNSLNLQMNSLRVEDTAIY |
| without signal | YCARDRPVAGASALWGQGTLVTVSS |
| sequence | |
| SEQ ID NO: 82 | DIQMTQSPSTLSASVGDRVTITCRASQSISGWLAWYQQKPGKAPKLLIY |
| 3B6 V_{L}- | KASSLESGVPSRFSGSGSGTEFTLTISSLQPDDFATYYCQQYYGSSRTFG |
| without signal | QGTNVEIK |
| sequence | |
| SEQ ID NO: 83 | EVRLVESGGGLVQPGGSLRLSCAASGDIISTHWMSWVRQAPGKGLEW |
| 8B1 V_{H}- | VANIKQDGSEKYYVDSVKGRFTISRDNAKNSLYLQMNTLRVEDTAIYY |
| without signal | CTRDRPVAGASALWGQGTLVTVSS |
| sequence | |
| SEQ ID NO: 84 | DIQMTQSPSTLSASVGDRVTITCRASQSISGWLAWYQQKPGKAPKLLIY |
| 8B1 V_{L}- | KASSLESGVPLRFSGSGSGTEFTLTISSLQPDDFATYYCQQYYGSSRTFG |
| without signal | QGTNVEIK |
| sequence | |
| SEQ ID NO: 85 | EVQLVESGGGLVQPGGSLRLSCAASGGIISSSWMSWVRQAPGKGLEWV |
| 4A3 V_{H}- | ANIKQDGSEKYYVDSVKGRFTISRDNAKDLLYLQMNSLRVEDTALYYC |
| without signal | ARDRPVAGASALWGQGTLVTVSS |
| sequence | |
| SEQ ID NO: 86 | DIQMTQSPSTLSASVGDRVTITCRASQSISGWLAWYQQKPGKAPKLLIY |
| 4A3 V_{L}- | KASSLESGVPSRFSGSGSGTEFTLTISSLQPDDFATYYCQQYYGSSRTFG |
| without signal | QGTNVEIK |
| sequence | |
| SEQ ID NO: 87 | EVQLVESGGGLVQPGGSLRLSCAASGGIISTYWMSWVRQAPGKGLEW |
| 9H9 V_{H}- | VANIKQDGSEKYYVDSVKGRFTISRDNAKNSLYLQMNSLRVEDTAMY |
| without signal | YCARDRPVAGASALWGQGTLVTVSS |
| sequence | |
| SEQ ID NO: 88 | DIQMTQSPSTLSASVGDRVTITCRASQSISGWLAWYQQKPGKAPKLLIY |
| 9H9 V_{L}- | KASSLESGVPSRFSGSGSGTEFTLTISSLQPDDFATYYCQQYYGSSRTFG |
| without signal | QGTNVEIK |
| sequence | |
| SEQ ID NO: 89 | ATGGAATTGGGGCTGAGCTGNGTTTTCCTTGTTGCTATTTTAGAAGG |
| 7H7 V_{H} DNA | TGTCCAGTGTGAGGTGCAACTGGTGGAGTCTGGGGGAGGCTTGGTC |
| Sequence-with | CAGCCTGGGGGGTCCCTGAGACTCTCCTGTGCAGCCTCTGGAGGCA |
| signal sequence | CCATTAGTACCTCTTGGATGAGCTGGGTCCGCCAGGCTCCAGGGAA |
| | GGGGCTGGAATGGGTGGCCAACATAAAGCAAGATGGAAGTGAGAA |
| | ATATTATGTGGACTCTGTGAAGGGCCGATTCACCATCTCCAGAGACA |
| | ACGCCAAGAACTCACTGTATTTGCAAATGAACAGCCTGAGAGTCGA |
| | GGACACGGCTATATATTACTGTGCGAGAGATCGTCCAGTGGCTGGT |
| | GCGTCGGCCCTCTGGGGCCAGGGAACCCTGGTCACCGTCTCCTCA |
| SEQ ID NO: 90 | ATGAGGGTCCCCGCTCAGCTCCTGGGGCTCCTGCTGCTCTGGCTCCC |
| 7H7 V_{L} DNA | AGGTGCCAAATGTGACATCCAGATGACCCAGTCTCCTTCCACCCTGT |
| Sequence-with | CTGCATCTGTAGGAGACAGAGTCACCATCACTTGCCGGGCCAGTCA |
| signal sequence | GAGTATAAGTGGCTGGTTGGCCTGGTATCAGCAGAAACAAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 91 | ATGGAATTGGGGCTGAGCTGGGTTTTCCTTGTTGCTATTTTAGAAGG |
| 1B3 V_{H} DNA | TGTCCAGTGTGAGGTGCAACTGGTGGAGTCTGGGGGAGGCTTGGTC |
| Sequence-with | CAGCCTGGGGGGTCCCTGAGACTCTCCTGTGCAGCCTCTGGAGGCA |
| signal sequence | CCATTAGTACCTCTTGGATGAGCTGGGTCCGCCAGGCTCCAGGGAA |
| | GGGGCTGGAATGGGTGGCCAACATAAAGCAAGATGGAAGTGAGAA |
| | ATATTATGTGGACTCTGTGAAGGGCCGATTCACCATCTCCAGAGACA |
| | ACGCCAAGAACTCACTGTATTTGCAAATGAACAGCCTGAGAGTCGA |
| | AGACACGGCTATGTATTACTGTGCGAGAGATCGTCCAGTGGCTGGT |
| | GCGTCGGCCCTCTGGGGCCAGGGAACCCTGGTCACCGTCTCCTCA |
| SEQ ID NO: 92 | ATGAGGGTCCCCGCTCAGCTCCTGGGGCTCCTGCTGCTCTGGCTCCC |
| 1B3 V_{L} DNA | AGGTGCCAAATGTGACATCCAGATGACCCAGTCTCCTTCCACCCTGT |
| Sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 93 | ATGGAATTGGGGCTGAGCTGGGTTTTCCTTGTTGCTATTTTAGAAGG |
| 3B6 V_{H} DNA | TGTCCAGTGTGAGGTGCAACTGGTGGAGTCTGGGGGAGGCTTGGTC |
| Sequence-with | CAGCCTGGGGGGTCCCTGAGACTCTCCTGTGCAGCCTCTGGAGGCA |
| signal sequence | CAACCAGTACCTATTGGATGAGCTGGGTCCGCCAGGCTCCAGGGAA |
| | GGGGCTGGAATGGGTGGCCAACATAAAGCAAGATGGAAGTGAGAA |
| | ATATTATGTGGACTCTGTGAAGGGCCGATTCACCATCTCCAGAGACA |
| | ACGCCAAGAACTCACTGAATTTGCAAATGAACAGCCTGAGAGTCGA |
| | GGACACGGCTATATATTACTGTGCGAGAGATCGTCCAGTGGCTGGT |
| | GCGTCGGCCCTCTGGGGCCAGGGAACCCTGGTCACCGTCTCCTCA |
| SEQ ID NO: 94 | ATGAGGGTCCCCGCTCAGCTCCTGGGGCTCCTGCTGCTCTGGCTCCC |
| 3B6 V_{L} DNA | AGGTGCCAAATGTGACATCCAGATGACCCAGTCTCCTTCCACCCTGT |
| Sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 95 | ATGGAATTGGGGCTGAGCTGGGTTTTCCTTGTTGCTATTTTAGAAGG |
| 8B1 V_{H} DNA | TGTCAAGTGTGAGGTGCGACTGGTGGAGTCTGGGGGAGGCTTGGTC |
| Sequence-with | CAGCCTGGGGGGTCCCTGAGACTCTCCTGTGCAGCCTCTGGAGACAT |
| signal sequence | AATTAGTACCCATTGGATGAGCTGGGTCCGCCAGGCTCCAGGGAAG |
| | GGGCTGGAATGGGTGGCCAACATAAAACAAGATGGAAGTGAGAAG |
| | TATTATGTGGACTCTGTGAAGGGCCGATTCACCATCTCCAGAGACAA |
| | CGCCAAGAACTCACTGTATTTGCAAATGAACACCCTGAGAGTCGAG |
| | GACACGGCTATATATTACTGTACGAGAGATCGTCCAGTGGCTGGTG |
| | CGTCGGCCCTCTGGGGCCAGGGAACCCTGGTCACCGTCTCCTCA |
| SEQ ID NO: 96 | ATGAGGGTCCCCGCTCAGCTCCTGGGGCTCCTGCTGCTCTGGCTCCC |
| 8B1 V_{L} DNA | AGGTGCCAAATGTGACATCCAGATGACCCAGTCTCCTTCCACCCTGT |
| Sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATTAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 97 | ATGGAATTGGGGCTGAGCTGGGTTTTCCTTGTTGCTATTTTAGAAGG |
| 4A3 V_{H} DNA | TGTCCAGTGTGAGGTGCAACTGGTGGAGTCTGGGGGAGGCTTGGTC |
| Sequence-with | CAGCCGGGGGGGTCCCTGAGACTCTCCTGTGCAGCCTCTGGAGGCA |
| signal sequence | TCATTAGTTCCTCTTGGATGAGCTGGGTCCGCCAGGCTCCAGGGAAG |
| | GGGCTGGAATGGGTGGCCAACATAAAGCAAGATGGAAGTGAGAAA |
| | TATTATGTGGACTCTGTGAAGGGCCGATTCACCATCTCCAGAGACAA |
| | CGCCAAAGACTTACTGTATTTGCAAATGAACAGCCTGAGAGTCGAG |
| | GACACGGCTTTATATTACTGTGCGAGAGATCGTCCAGTGGCTGGTGC |
| | GTCGGCCCTCTGGGGCCAGGGAACCCTGGTCACCGTCTCCTCT |
| SEQ ID NO: 98 | ATGAGGGTCCCCGCTCAGCTCCTGGGGCTCCTGCTGCTCTGGCTCCC |
| 4A3 V_{L} DNA | AGGTGCCAAATGTGACATCCAGATGACCCAGTCTCCTTCCACCCTGT |
| Sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 99 | ATGGAATTGGGGCTGAGCTGGGTTTTCCTTGTTGCTATTTTAGAAGG |
| 9H9 V_{H} DNA | TGTCCAGTGTGAGGTGCAACTGGTGGAGTCTGGGGGAGGCTTGGTC |
| Sequence-with | CAGCCTGGGGGGTCCCTGAGACTCTCCTGTGCAGCCTCTGGAGGCAT |
| signal sequence | CATTAGTACCTATTGGATGAGCTGGGTCCGCCAGGCTCCAGGGAAG |
| | GGGCTGGAATGGGTGGCCAACATAAAGCAAGATGGAAGTGAGAAA |
| | TATTATGTGGACTCTGTGAAGGGCCGATTCACCATCTCCAGAGACAA |
| | CGCCAAGAACTCACTGTATTTGCAAATGAACAGCCTGAGAGTCGAG |
| | GACACGGCTATGTATTACTGTGCGAGAGATCGTCCAGTGGCTGGTG |
| | CGTCGGCCCTCTGGGGCCAGGGAACCCTGGTCACCGTCTCCTCA |
| SEQ ID NO: 100 | ATGAGGGTCCCCGCTCAGCTCCTGGGGCTCCTGCTGCTCTGGCTCCC |
| 9H9 V_{L} DNA | AGGTGCCAAATGTGACATCCAGATGACCCAGTCTCCTTCCACCCTGT |
| Sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 101 | GAGGTGCAACTGGTGGAGTCTGGGGGAGGCTTGGTCCAGCCTGGGG |
| 7H7 V_{H} DNA | GGTCCCTGAGACTCTCCTGTGCAGCCTCTGGAGGCACCATTAGTACC |
| Sequence- | TCTTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGTATTTGCAAATGAACAGCCTGAGAGTCGAGGACACGGCTA |
| | TATATTACTGTGCGAGAGATCGTCCAGTGGCTGGTGCGTCGGCCCTC |
| | TGGGGCCAGGGAACCCTGGTCACCGTCTCCTCA |
| SEQ ID NO: 102 | GACATCCAGATGACCCAGTCTCCTTCCACCCTGTCTGCATCTGTAGG |
| 7H7 V_{L} DNA | AGACAGAGTCACCATCACTTGCCGGGCCAGTCAGAGTATAAGTGGC |
| Sequence- | TGGTTGGCCTGGTATCAGCAGAAACAAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 103 | GAGGTGCAACTGGTGGAGTCTGGGGGAGGCTTGGTCCAGCCTGGGG |
| 1B3 V_{H} DNA | GGTCCCTGAGACTCTCCTGTGCAGCCTCTGGAGGCACCATTAGTACC |
| Sequence- | TCTTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGTATTTGCAAATGAACAGCCTGAGAGTCGAAGACACGGCTA |
| | TGTATTACTGTGCGAGAGATCGTCCAGTGGCTGGTGCGTCGGCCCTC |
| | TGGGGCCAGGGAACCCTGGTCACCGTCTCCTCA |
| SEQ ID NO: 104 | GACATCCAGATGACCCAGTCTCCTTCCACCCTGTCTGCATCTGTAGG |
| 1B3 V_{L} DNA | AGACAGAGTCACCATCACTTGCCGGGCCAGTCAGAGTATTAGTGGC |
| Sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 105 | GAGGTGCAACTGGTGGAGTCTGGGGGAGGCTTGGTCCAGCCTGGGG |
| 3B6 V_{H} DNA | GGTCCCTGAGACTCTCCTGTGCAGCCTCTGGAGGCACAACCAGTACC |
| Sequence- | TATTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGAATTTGCAAATGAACAGCCTGAGAGTCGAGGACACGGCTA |
| | TATATTACTGTGCGAGAGATCGTCCAGTGGCTGGTGCGTCGGCCCTC |
| | TGGGGCCAGGGAACCCTGGTCACCGTCTCCTCA |
| SEQ ID NO: 106 | GACATCCAGATGACCCAGTCTCCTTCCACCCTGTCTGCATCTGTAGG |
| 3B6 V_{L} DNA | AGACAGAGTCACCATCACTTGCCGGGCCAGTCAGAGTATTAGTGGC |
| Sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 107 | GAGGTGCGACTGGTGGAGTCTGGGGGAGGCTTGGTCCAGCCTGGGG |
| 8B1 V_{H} DNA | GGTCCCTGAGACTCTCCTGTGCAGCCTCTGGAGACATAATTAGTACC |
| Sequence- | CATTGGATGAGCTGGGTCCGCCAGGCTCCAGGGAAGGGGCTGGAAT |
| without signal | GGGTGGCCAACATAAAACAAGATGGAAGTGAGAAGTATTATGTGGA |
| sequence | CTCTGTGAAGGGCCGATTCACCATCTCCAGAGACAACGCCAAGAAC |
| | TCACTGTATTTGCAAATGAACACCCTGAGAGTCGAGGACACGGCTA |
| | TATATTACTGTACGAGAGATCGTCCAGTGGCTGGTGCGTCGGCCCTC |
| | TGGGGCCAGGGAACCCTGGTCACCGTCTCCTCA |
| SEQ ID NO: 108 | GACATCCAGATGACCCAGTCTCCTTCCACCCTGTCTGCATCTGTAGG |
| 8B1 V_{L} DNA | AGACAGAGTCACCATCACTTGCCGGGCCAGTCAGAGTATTAGTGGC |
| Sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATTAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 109 | GAGGTGCAACTGGTGGAGTCTGGGGGAGGCTTGGTCCAGCCGGGGG |
| 4A3 V_{H} DNA | GGTCCCTGAGACTCTCCTGTGCAGCCTCTGGAGGCATCATTAGTTCC |
| Sequence- | TCTTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAAGAC |
| | TTACTGTATTTGCAAATGAACAGCCTGAGAGTCGAGGACACGGCTTT |
| | ATATTACTGTGCGAGAGATCGTCCAGTGGCTGGTGCGTCGGCCCTCT |
| | GGGGCCAGGGAACCCTGGTCACCGTCTCCTCT |
| SEQ ID NO: 110 | GACATCCAGATGACCCAGTCTCCTTCCACCCTGTCTGCATCTGTAGG |
| 4A3 V_{L} DNA | AGACAGAGTCACCATCACTTGCCGGGCCAGTCAGAGTATTAGTGGC |
| Sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 111 | GAGGTGCAACTGGTGGAGTCTGGGGGAGGCTTGGTCCAGCCTGGGG |
| 9H9 V_{H} DNA | GGTCCCTGAGACTCTCCTGTGCAGCCTCTGGAGGCATCATTAGTACC |
| Sequence- | TATTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGTATTTGCAAATGAACAGCCTGAGAGTCGAGGACACGGCTA |
| | TGTATTACTGTGCGAGAGATCGTCCAGTGGCTGGTGCGTCGGCCCTC |
| | TGGGGCCAGGGAACCCTGGTCACCGTCTCCTCA |
| SEQ ID NO: 112 | GACATCCAGATGACCCAGTCTCCTTCCACCCTGTCTGCATCTGTAGG |
| 9H9 V_{L} DNA | AGACAGAGTCACCATCACTTGCCGGGCCAGTCAGAGTATTAGTGGC |
| Sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 113 | TGSISGY |
| 3C2 V_{H} CDR1 | |
| (Chothia) | |
| SEQ ID NO: 114 | YYSGS |
| 3C2 V_{H} CDR2 | |
| (Chothia) | |
| SEQ ID NO: 115 | YPLIRGAFDY |
| 3C2 V_{H} CDR3 | |
| (Chothia) | |
| SEQ ID NO: 116 | RSSQNLLHTNGYNYLD |
| 3C2 V_{L} CDR1 | |
| (Chothia) | |
| SEQ ID NO: 117 | LGSNRAS |
| 3C2 V_{L} CDR2 | |
| (Chothia) | |
| SEQ ID NO: 118 | MQALQTPLT |
| 3C2 V_{L} CDR3 | |
| (Chothia) | |
| SEQ ID NO: 119 | GYTFTGY |
| 2B3 V_{H} CDR1 | |
| (Chothia) | |
| SEQ ID NO: 120 | NPNSGG |
| 2B3 V_{H} CDR2 | |
| (Chothia) | |
| SEQ ID NO: 121 | DRLVLPWFGEIFPDAFDI |
| 2B3 V_{H} CDR3 | |
| (Chothia) | |
| SEQ ID NO: 122 | RASQSIRSNLA |
| 2B3 V_{L} CDR1 | |
| (Chothia) | |
| SEQ ID NO: 123 | GASTRAT |
| 2B3 V_{L} CDR2 | |
| (Chothia) | |
| SEQ ID NO: 124 | QQYNNWPLT |
| 2B3 V_{L} CDR3 | |
| (Chothia) | |
| SEQ ID NO: 125 | GGTISTS |
| 7H7 V_{H} CDR1 | |
| (Chothia) | |
| SEQ ID NO: 126 | KQDGSE |
| 7H7 V_{H} CDR2 | |
| (Chothia) | |
| SEQ ID NO: 127 | DRPVAGASAL |
| 7H7 V_{H} CDR3 | |
| (Chothia) | |
| SEQ ID NO: 128 | RASQSISGWLA |
| 7H7 V_{L} CDR1 | |
| (Chothia) | |
| SEQ ID NO: 129 | KASSLES |
| 7H7 V_{L} CDR2 | |
| (Chothia) | |
| SEQ ID NO: 130 | QQYYGSSRT |
| 7H7 V_{L} CDR3 | |
| (Chothia) | |
| SEQ ID NO: 131 | GGTISTS |
| 1B3 V_{H} CDR1 | |
| (Chothia) | |
| SEQ ID NO: 132 | KQDGSE |
| 1B3 V_{H} CDR2 | |
| (Chothia) | |
| SEQ ID NO: 133 | DRPVAGASAL |
| 1B3 V_{H} CDR3 | |
| (Chothia) | |
| SEQ ID NO: 134 | RASQSISGWLA |
| 1B3 V_{L} CDR1 | |
| (Chothia) | |
| SEQ ID NO: 135 | KASSLES |
| 1B3 V_{L} CDR2 | |
| (Chothia) | |
| SEQ ID NO: 136 | QQYYGSSRT |
| 1B3 V_{L} CDR3 | |
| (Chothia) | |
| SEQ ID NO: 137 | GGTTSTY |
| 3B6 V_{H} CDR1 | |
| (Chothia) | |
| SEQ ID NO: 138 | KQDGSE |
| 3B6 V_{H} CDR2 | |
| (Chothia) | |
| SEQ ID NO: 139 | DRPVAGASAL |
| 3B6 V_{H} CDR3 | |
| (Chothia) | |
| SEQ ID NO: 140 | RASQSISGWLA |
| 3B6 V_{L} CDR1 | |
| (Chothia) | |
| SEQ ID NO: 141 | KASSLES |
| 3B6 V_{L} CDR2 | |
| (Chothia) | |
| SEQ ID NO: 142 | QQYYGSSRT |
| 3B6 V_{L} CDR3 | |
| (Chothia) | |
| SEQ ID NO: 143 | GDIISTH |
| 8B1 V_{H} CDR1 | |
| (Chothia) | |
| SEQ ID NO: 144 | KQDGSE |
| 8B1 V_{H} CDR2 | |
| (Chothia) | |
| SEQ ID NO: 145 | DRPVAGASAL |
| 8B1 V_{H} CDR3 | |
| (Chothia) | |
| SEQ ID NO: 146 | RASQSISGWLA |
| 8B1 V_{L} CDR1 | |
| (Chothia) | |
| SEQ ID NO: 147 | KASSLES |
| 8B1 V_{L} CDR2 | |
| (Chothia) | |
| SEQ ID NO: 148 | QQYYGSSRT |
| 8B1 V_{L} CDR3 | |
| (Chothia) | |
| SEQ ID NO: 149 | GGIISSS |
| 4A3 V_{H} CDR1 | |
| (Chothia) | |
| SEQ ID NO: 150 | KQDGSE |
| 4A3 V_{H} CDR2 | |
| (Chothia) | |
| SEQ ID NO: 151 | DRPVAGASAL |
| 4A3 V_{H} CDR3 | |
| (Chothia) | |
| SEQ ID NO: 152 | RASQSISGWLA |
| 4A3 V_{L} CDR1 | |
| (Chothia) | |
| SEQ ID NO: 153 | KASSLES |
| 4A3 V_{L} CDR2 | |
| (Chothia) | |
| SEQ ID NO: 154 | QQYYGSSRT |
| 4A3 V_{L} CDR3 | |
| (Chothia) | |
| SEQ ID NO: 155 | GGIISTY |
| 9H9 V_{H} CDR1 | |
| (Chothia) | |
| SEQ ID NO: 156 | KQDGSE |
| 9H9 V_{H} CDR2 | |
| (Chothia) | |
| SEQ ID NO: 157 | DRPVAGASAL |
| 9H9 V_{H} CDR3 | |
| (Chothia) | |
| SEQ ID NO: 158 | RASQSISGWLA |
| 9H9 V_{L} CDR1 | |
| (Chothia) | |
| SEQ ID NO: 159 | KASSLES |
| 9H9 V_{L} CDR2 | |
| (Chothia) | |
| SEQ ID NO: 160 | QQYYGSSRT |
| 9H9 V_{L} CDR3 | |
| (Chothia) | |
| SEQ ID NO: 161 | GYY(W, I)(S, H) |
| CD27 antibody | |
| V_{H} CDR1 | |
| consensus | |
| sequence | |
| (Kabat) | |
| SEQ ID NO: 162 | (-, W)(Y, I)N(Y, P)(Y, N)SG(S, G)TN(Y,S )(N, A)(P, Q)(S, K)(L, F)(K, Q) |
| CD27 antibody | (S, D) |
| V_{H} CDR2 | |
| consensus | |
| sequence | |
| (Kabat) | |
| SEQ ID NO: 163 | (-, D)(-, R)(-, L)(-, V)(-, L)(-, P)(-, W)(-, F)(-, G)(-, E)(-, I)(- |
| CD27 antibody | Y, F)P(L, -)(I, -)(R, -)(G, D)AFD(Y, I) |
| V_{H} CDR3 | |
| consensus | |
| sequence | |
| (Kabat) | |
| SEQ ID NO: 164 | R(S, A) |
| CD27 antibody | SQ(-, S)(-, I)(-, R)(-, S)NL(L, A)(H, -)(T, -)(N, -)(G, -)(Y, -)(N, -) |
| V_{L} CDR1 | (Y, -)(L, -)(D, -) |
| consensus | |
| sequence | |
| (Kabat) | |
| SEQ ID NO: 165 | (L, G)(G, A)S(N, T)RA(S, T) |
| CD27 antibody | |
| V_{L} CDR2 | |
| consensus | |
| sequence | |
| (Kabat) | |
| SEQ ID NO: 166 | (M, Q)Q(A, Y)(L, N)(Q, N)(T, W)PLT |
| CD27 antibody | |
| V_{L} CDR3 | |
| consensus | |
| sequence | |
| (Kabat) | |
| SEQ ID NO: 167 | (T, S)(S, Y, H)WMS |
| PD-L1 antibody | |
| V_{H} CDR1 | |
| consensus | |
| sequence | |
| (Kabat) | |
| SEQ ID NO: 168 | NIKQDGSEKYYVDSVKG |
| PD-L1 antibody | |
| V_{H} CDR2 | |
| consensus | |
| sequence | |
| (Kabat) | |
| SEQ ID NO: 169 | DRPVAGASAL |
| PD-L1 antibody | |
| V_{H} CDR3 | |
| consensus | |
| sequence | |
| (Kabat) | |
| SEQ ID NO: 170 | RASQSISGWLA |
| PD-L1 antibody | |
| V_{L} CDR1 | |
| consensus | |
| sequence | |
| (Kabat) | |
| SEQ ID NO: 171 | KASSLES |
| PD-L1 antibody | |
| V_{L} CDR2 | |
| consensus | |
| sequence | |
| (Kabat) | |
| SEQ ID NO: 172 | QQYYGSSRT |
| PD-L1 antibody | |
| V_{L} CDR3 | |
| consensus | |
| sequence | |
| (Kabat) | |
| SEQ ID NO: 173 | MARPHPWWLCVLGTLVGLSATPAPKSCPERHYWAQGKLCCQMCEPG |
| CD27 amino | TFLVKDCDQHRKAAQCDPCIPGVSFSPDHHTRPHCESCRHCNSGLLVR |
| acid sequence | NCTITANAECACRNGWQCRDKECTECDPLPNPSLTARSSQALSPHPQPT |
| (Accession No. | HLPYVSEMLEARTAGHMQTLADFRQLPARTLSTHWPPQRSLCSSDFIRI |
| AAH12160.1) | LVIFSGMFLVFTLAGALFLHQRRKYRSNKGESPVEPAEPCRYSCPREEE |
| | GSTIPIQEDYRKPEPACSP |
| SEQ ID NO: 174 | MPEEGSGCSVRRRPYGCVLRAALVPLVAGLVICLVVCIQRFAQAQQQL |
| CD70 amino | PLESLGWDVAELQLNHTGPQQDPRLYWQGGPALGRSFLHGPELDKGQ |
| acid sequence | LRIHRDGIYMVHIQVTLAICSSTTASRHHPTTLAVGICSPASRSISLLRLSF |
| (Accession No. | HQGCTIASQRLTPLARGDTLCTNLTGTLLPSRNTDETFFGVQWVRP |
| NP_001243) | |
| SEQ ID NO: 175 | MQIPQAPWPVVWAVLQLGWRPGWFLDSPDRPWNPPTFSPALLVVTEG |
| PD1 amino acid | DNATFTCSFSNTSESFVLNWYRMSPSNQTDKLAAFPEDRSQPGQDCRF |
| sequence | RVTQLPNGRDFHMSVVRARRNDSGTYLCGAISLAPKAQIKESLRAELR |
| (Accession No. | VTERRAEVPTAHPSPSPRPAGQFQTLVVGVVGGLLGSLVLLVWVLAVI |
| NP_005009) | CSRAARGTIGARRTGQPLKEDPSAVPVFSVDYGELDFQWREKTPEPPVP |
| | CVPEQTEYATIVFPSGMGTSSPARRGSADGPRSAQPLRPEDGHCSWPL |
| SEQ ID NO: 176 | MRIFAVFIFMTYWHLLNAPYNKINQRILVVDPVTSEHELTCQAEGYPKA |
| PD-L1 amino | EVIWTSSDHQVLSGKTTTTNSKREEKLFNVTSTLRINTTTNEIFYCTFRR |
| acid sequence | LDPEENHTAELVIPELPLAHPPNERTHLVILGAILLCLGVALTFIFRLRKG |
| (isoform b | RMMDVKKCGIQDTNSKKQSDTHLEET |
| precursor) | |
| (Accession No. | |
| NP_001254635) | |
| SEQ ID NO: 177 | QVQLVQSGAEVKKPGASVKVSCKASGYTFTGYYIHWVRQAPGQGLE |
| 2B3 V_{H} | WMGWINPNSGGTNSAQKFQDRVTITRDTSINTAYMELSRLRSDDTAVY |
| (modified) | FCARDRLVLPWFGEIFPDAFDIWGQGTLVTVSS |
| SEQ ID NO: 178 | EIVMTQSPATLSVSPGERATLSCRASQSIRSNLAWYQQKPGQAPRLLIY |
| 2B3 V_{L} | GASTRATGIPARFSGSGSGTEFTLTISSLQSEDFAVYYCQQYNNWPLTFG |
| (modified) | GGTKVEIK |
| SEQ ID NO: 179 | EVQLVESGGGLVQPGGSLRLSCAASGGIISTYWMSWVRQAPGKGLEW |
| Full 9H9-2B3 | VANIKQDGSEKYYVDSVKGRFTISRDNAKNSLYLQMNSLRVEDTAMY |
| (CDX-527) | YCARDRPVAGASALWGQGTLVTVSSASTKGPSVFPLAPSSKSTSGGT |
| heavy chain | AALGCLVKDYFPEPVTVSWNSGALTSGVHTFPAVLQSSGLYSLSSV |
| sequence was as | VTVPSSSLGTQTYICNVNHKPSNTKVDKKVEPKSCDKTHTCPPCPA |
| follows (with the | PELLGGPSVFLFPPKPKDTLMISRTPEVTCVVVDVSHEDPEVKFNW |
| IgG1 constant | YVDGVEVHNAKTKPREEQYNSTYRVVSVLTVLHQDWLNGKEYKC |
| region sequence | KVSNKALPAPIEKTISKAKGQPREPQVYTLPPSRDELTKNQVSLTCL |
| shown in bold) | VKGFYPSDIAVEWESNGQPENNYKTTPPVLDSDGSFFLYSKLTVDK |
| | SRWQQGNVFSCSVMHEALHNHYTQKSLSLSPGKGSSGGGGSEIVMT |
| | QSPATLSVSPGERATLSCRASQSIRSNLAWYQQKPGQAPRLLIYGASTR |
| | ATGIPARFSGSGSGTEFTLTISSLQSENFAVYYCQQYNNWPLTFGCGTK |
| | VEIKGGGGSGGGGSGGGGSGGGGSQVQLVQSGAEVKKPGASVKVSCK |
| | ASGYTFTGYYIHWVRQAPGQCLEWMGWINPNSGGTNSAQKFQDRVTI |
| | TRVTSINTAYMELSRLRSDDTAVYFCARDRLVLPWFGEIFPDAFDIWGQ |
| | GTLVTVSS |
| SEQ ID NO: 180 | DIQMTQSPSTLSASVGDRVTITCRASQSISGWLAWYQQKPGKAPKLLIY |
| 9H9-2B3 (CDX- | KASSLESGVPSRFSGSGSGTEFTLTISSLQPDDFATYYCQQYYGSSRTFG |
| 527) light chain | QGTNVEIKRTVAAPSVFIFPPSDEQLKSGTASVVCLLNNFYPREAKV |
| sequence was as | QWKVDNALQSGNSQESVTEQDSKDSTYSLSSTLTLSKADYEKHKVY |
| follows (with the | ACEVTHQGLSSPVTKSFNRGEC |
| constant region | |
| sequence shown | |
| in bold) | |
| SEQ ID NO: 181 | EVQLVESGGGLVQPGGSLRLSCAASGGIISTYWMSWVRQAPGKGLEW |
| Full 9H9- | VANIKQDGSEKYYVDSVKGRFTISRDNAKNSLYLQMNSLRVEDTAMY |
| 2B3(DD) heavy | YCARDRPVAGASALWGQGTLVTVSSASTKGPSVFPLAPSSKSTSGGT |
| chain sequence | AALGCLVKDYFPEPVTVSWNSGALTSGVHTFPAVLQSSGLYSLSSV |
| was as follows | VTVPSSSLGTQTYICNVNHKPSNTKVDKKVEPKSCDKTHTCPPCPA |
| (with the IgG1 | PELLGGPSVFLFPPKPKDTLMISRTPEVTCVVVDVSHEDPEVKFNW |
| constant | YVDGVEVHNAKTKPREEQYNSTYRVVSVLTVLHQDWLNGKEYKC |
| “backbone” | KVSNKALPAPIEKTISKAKGQPREPQVYTLPPSRDELTKNQVSLTCL |
| sequence shown | VKGFYPSDIAVEWESNGQPENNYKTTPPVLDSDGSFFLYSKLTVDK |
| in bold) | SRWQQGNVFSCSVMHEALHNHYTQKSLSLSPGKGSSGGGGSEIVMT |
| | QSPATLSVSPGERATLSCRASQSIRSNLAWYQQKPGQAPRLLIYGASTR |
| | ATGIPARFSGSGSGTEFTLTISSLQSEDFAVYYCQQYNNWPLTFGCGTK |
| | VEIKGGGGSGGGGSGGGGSGGGGSQVQLVQSGAEVKKPGASVKVSCK |
| | ASGYTFTGYYIHWVRQAPGQCLEWMGWINPNSGGTNSAQKFQDRVTI |
| | TRDTSINTAYMELSRLRSDDTAVYFCARDRLVLPWFGEIFPDAFDIWGQ |
| | GTLVTVSS |
| SEQ ID NO: 182 | DIQMTQSPSTLSASVGDRVTITCRASQSISGWLAWYQQKPGKAPKLLIY |
| 9H9-2B3(DD) | KASSLESGVPSRFSGSGSGTEFTLTISSLQPDDFATYYCQQYYGSSRTFG |
| light chain | QGTNVEIKRTVAAPSVFIFPPSDEQLKSGTASVVCLLNNFYPREAKV |
| sequence was as | QWKVDNALQSGNSQESVTEQDSKDSTYSLSSTLTLSKADYEKHKVY |
| follows (with the | ACEVTHQGLSSPVTKSFNRGEC |
| constant region | |
| shown in bold) | |
| SEQ ID NO: 183 | TPAPKSCPER HYWAQGKLCC QMCEPGTFLV KDCDQHRKAA |
| ECD of huCD27 | QCDPCIPGVS FSPDHHTRPH CESCRHCNSG LLVRNCTITA |
| | NAECACRNGW QCRDKECTEC DPLPNPSLTA RSSQALSPHP |
| | QPTHLPYVSE MLEARTAGHM QTLADFRQLP ARTLSTHWPP |
| | QRSLCSSD |
| SEQ ID NO: 184 | TPAPKSCPER HYWAQGKLCC QMCEPGTFLV KDCDQHRKAA |
| ECD of huCD27 | QCDPCIPGVS FSPDHHTRPH CESCRHCNSG LLVRNCTITA |
| mutated | NAECSCAAAW QCRDKECTEC DPLPNPSLTA RSSQALSPHP |
| | QPTHLPYVSE MLEARTAGHM QTLADFRQLP ARTLSTHWPP |
| | QRSLCSSD |
| SEQ ID NO: 185 | GAAGTGCAACTGGTGGAGTCGGGTGGTGGACTCGTGCAGCCCGGCG |
| NA sequence of | GATCCCTGAGACTCTCTTGTGCCGCATCGGGCGGCATTATTAGCACT |
| 9H9x2B3(DD) | TACTGGATGTCATGGGTCAGACAGGCACCGGGAAAGGGCTTGGAAT |
| variable domain | GGGTGGCGAATATCAAGCAGGATGGATCCGAGAAGTACTACGTGGA |
| | CTCCGTGAAGGGCAGATTCACCATTTCCCGGGACAACGCCAAGAAC |
| | TCGCTCTATCTGCAAATGAACTCGTTGCGGGTGGAAGATACTGCCAT |
| | GTACTACTGCGCCCGGGACCGGCCTGTGGCCGGGGCGTCGGCCCTC |
| | TGGGGCCAGGGCACTCTGGTCACCGTGTCCTCT |
| SEQ ID NO: 186 | GGCTCCAGCGGGGGTGGCGGTTCCGAGATCGTGATGACTCAGAG |
| NA sequence of | CCCGGCAACCCTGTCCGTGTCTCCGGGGGAGCGGGCTACTCTTTCCT |
| 9H9x2B3(DD) | GCCGGGCATCCCAGTCCATCCGGTCGAACCTTGCGTGGTACCAACA |
| scFv domain | GAAGCCTGGACAGGCGCCCCGCCTGCTGATCTACGGGGCGTCGACT |
| (connector and | AGGGCCACCGGCATCCCGGCCCGCTTCTCCGGGTCCGGATCCGGCA |
| linker sequences | CCGAATTCACCCTCACCATCTCGAGCCTGCAGTCCGAAAACTTCGCC |
| are shown in | GTCTACTACTGCCAGCAGTACAACAACTGGCCGCTGACATTCGGAT |
| bold) | GCGGAACCAAAGTGGAAATCAAGGGCGGCGGCGGATCCGGCGGT |
| | GGCGGCAGCGGCGGTGGAGGATCCGGTGGCGGCGGTTCACAAG |
| | TGCAGCTGGTGCAGTCAGGCGCCGAAGTCAAGAAGCCCGGGGCCAG |
| | CGTGAAAGTCAGCTGCAAGGCTTCCGGATACACCTTCACGGGTTACT |
| | ACATTCACTGGGTTCGCCAAGCGCCCGGGCAGTGTCTGGAGTGGAT |
| | GGGATGGATCAACCCTAACTCGGGGGGAACCAACTCGGCCCAAAAG |
| | TTCCAGGACCGGGTCACCATTACAAGAGTCACGTCCATCAACACTG |
| | CCTACATGGAGTTGAGCCGGCTGCGATCAGACGACACCGCCGTGTA |
| | CTTCTGCGCGAGGGACCGCCTCGTCCTCCCGTGGTTTGGAGAGATCT |
| | TCCCGGATGCCTTCGACATTTGGGGACAGGGGACCCTCGTGACTGTG |
| | TCCAGC |
| SEQ ID NO: 187 | GATATCCAGATGACCCAGAGCCCGTCCACCCTTTCCGCGAGCGTCG |
| NA sequence of | GCGACAGAGTGACCATTACTTGTCGGGCCTCGCAAAGCATCTCCGG |
| the | CTGGCTGGCTTGGTACCAGCAAAAGCCTGGAAAGGCCCCTAAGCTG |
| 9H9x2B3(DD) | CTGATCTACAAGGCCTCATCCCTGGAGTCCGGAGTGCCTTCACGCTT |
| light chain | TTCGGGGAGCGGATCGGGGACTGAGTTCACCCTCACCATTTCCTCCC |
| variable domain | TGCAACCCGACGATTTCGCGACATACTACTGCCAGCAGTACTACGGT |
| | TCCTCGCGCACGTTCGGACAGGGCACTAACGTCGAGATCAAG (SEQ |
| | ID NO: 187) |

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A bispecific construct comprising an anti-CD27 binding domain linked to an anti-PD-L1 binding domain, wherein:
(i) the anti-CD27 binding domain comprises:
a. heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof, or
b. heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof; and
(ii) the anti-PD-L1 binding domain comprises:
a. a heavy chain variable region CDR1 comprising an amino acid sequence selected from the consensus sequence: (T,S)(S,Y,H)WMS (SEQ ID NO:167) or conservative sequence modifications thereof;
a heavy chain variable region CDR2 comprising SEQ ID NO:168 or conservative sequence modifications thereof;
a heavy chain variable region CDR3 comprising SEQ ID NO:169 or conservative sequence modifications thereof;
a light chain variable region CDR1 comprising SEQ ID NO:170 or conservative sequence modifications thereof;
a light chain variable region CDR2 comprising SEQ ID NO:171 or conservative sequence modifications thereof; and
a light chain variable region CDR3 comprising SEQ ID NO: 172 or conservative sequence modifications thereof;
b. heavy chain variable region CDR1, CDR2 and CDR3 as set forth in SEQ ID NOs: 29, 30, and 31, respectively, or conservative sequence modifications thereof, and light chain variable region CDR1, CDR2 and CDR3 as set forth in SEQ ID NOs: 32, 33, and 34, respectively, or conservative sequence modifications thereof;
c. heavy chain variable region CDR1, CDR2 and CDR3 as set forth in SEQ ID NOs: 35, 36, and 37, respectively, or conservative sequence modifications thereof, and light chain variable region CDR1, CDR2 and CDR3 as set forth in SEQ ID NOs: 38, 39, and 40, respectively, or conservative sequence modifications thereof;
d. heavy chain variable region CDR1, CDR2 and CDR3 as set forth in SEQ ID NOs: 41, 42, and 43, respectively, or conservative sequence modifications thereof, and light chain variable region CDR1, CDR2 and CDR3 as set forth in SEQ ID NOs: 44, 45, and 46, respectively, or conservative sequence modifications thereof;
e. heavy chain variable region CDR1, CDR2 and CDR3 as set forth in SEQ ID NOs: 47, 48, and 49, respectively, or conservative sequence modifications thereof, and light chain variable region CDR1, CDR2 and CDR3 as set forth in SEQ ID NOs: 50, 51, and 52, respectively, or conservative sequence modifications thereof;
f. heavy chain variable region CDR1, CDR2 and CDR3 as set forth in SEQ ID NOs: 53, 54, and 55, respectively, or conservative sequence modifications thereof, and light chain variable region CDR1, CDR2 and CDR3 as set forth in SEQ ID NOs: 56, 57, and 58, respectively, or conservative sequence modifications thereof; or
g. heavy chain variable region CDR1, CDR2 and CDR3 as set forth in SEQ ID NOs: 59, 60, and 61, respectively, or conservative sequence modifications thereof, and light chain variable region CDR1, CDR2 and CDR3 as set forth in SEQ ID NOs: 62, 63, and 64, respectively, or conservative sequence modifications thereof;
h. a heavy chain variable region comprising SEQ ID NO:77 and a light chain variable region comprising SEQ ID NO:78 or sequences at least 95% identical thereto;
i. a heavy chain variable region comprising SEQ ID NO:79 and a light chain variable region comprising SEQ ID NO:80 or sequences at least 95% identical thereto;
j. a heavy chain variable region comprising SEQ ID NO:81 and a light chain variable region comprising SEQ ID NO:82 or sequences at least 95% identical thereto;
k. a heavy chain variable region comprising SEQ ID NO:83 and a light chain variable region comprising SEQ ID NO:84 or sequences at least 95% identical thereto;
l. a heavy chain variable region comprising SEQ ID NO:85 and a light chain variable region comprising SEQ ID NO:86 or sequences at least 95% identical thereto; or
m. a heavy chain variable region comprising SEQ ID NO:87 and a light chain variable region comprising SEQ ID NO:88 or sequences at least 95% identical thereto.

2-40. (canceled)

41. The bispecific construct of claim 1, wherein (a) the anti-PD-L1 binding domain further comprises a human IgG1 constant domain or (b) the anti-CD27 binding domain further comprises a human IgG1 constant domain or (c) the anti-CD27 binding domain is linked to the C-terminus of the heavy chain of the anti-PD-L1 binding domain or (d) the anti-PD-L1 binding domain is linked to the C-terminus of the heavy chain of the anti-CD27 binding domain.

42. (canceled)

43. The bispecific construct of claim 1, wherein the anti-CD27 binding domain is a scFv or (b) the anti-PD-L1 binding domain is a scFv.

44. The bispecific construct of claim 1, wherein (a) the anti-PD-L1 binding domain and the anti-CD27 binding domain are genetically fused or (b) the anti-PD-L1 binding domain and the anti-CD27 binding domain are chemically conjugated.

45-57. (canceled)

58. An anti-CD27 antibody, or antigen-binding fragment thereof, wherein:
(a) the antibody binds to one or more residues within amino acid residues 85-89, of the extracellular domain (ECD) of human CD27 (SEQ ID NO: 183); and/or
(b) the antibody binds to the wildtype ECD of human CD27 (SEQ ID NO: 183), but does not bind to a mutated version of the wildtype ECD of human CD27 having the following amino acid substitutions: A85S, R87A, N88A, and G89A

59. (canceled)

60. An anti-CD27 antibody, or antigen-binding fragment thereof, comprising
(a) heavy chain variable region CDR1, CDR2 and CDR3 as set forth in SEQ ID NOs: 1, 2, and 3, respectively, or conservative sequence modifications thereof, and light chain variable region CDR1, CDR2 and CDR3 as set forth in SEQ ID NOs: 4, 5, and 6, respectively, or conservative sequence modifications thereof; or
(b) heavy chain variable region CDR1, CDR2 and CDR3 as set forth in SEQ ID NOs: 7, 8, and 9, respectively, or conservative sequence modifications thereof, and light chain variable region CDR1, CDR2 and CDR3 as set forth in SEQ ID NOs: 10, 11, and 12, respectively, or conservative sequence modifications thereof

61-64. (canceled)

65. A bispecific or multispecific construct comprising the anti-CD27 antibody, or antigen binding fragment thereof, of claim 60 linked to at least one addition binding domain.

66-68. (canceled)

69. An anti-PD-L1 antibody, or antigen-binding fragment thereof, comprising:
(a) heavy chain variable region CDR1, CDR2 and CDR3 as set forth in SEQ ID NOs: 29, 30, and 31, respectively, or conservative sequence modifications thereof, and light chain variable region CDR1, CDR2 and CDR3 as set forth in SEQ ID NOs: 32, 33, and 34, respectively, or conservative sequence modifications thereof;
(b) heavy chain variable region CDR1, CDR2 and CDR3 as set forth in SEQ ID NOs: 35, 36, and 37, respectively, or conservative sequence modifications thereof, and light chain variable region CDR1, CDR2 and CDR3 as set forth in SEQ ID NOs: 38, 39, and 40, respectively, or conservative sequence modifications thereof;
(c) heavy chain variable region CDR1, CDR2 and CDR3 as set forth in SEQ ID NOs: 41, 42, and 43, respectively, or conservative sequence modifications thereof, and light chain variable region CDR1, CDR2 and CDR3 as set forth in SEQ ID NOs: 44, 45, and 46, respectively, or conservative sequence modifications thereof;
(d) heavy chain variable region CDR1, CDR2 and CDR3 as set forth in SEQ ID NOs: 47, 48, and 49, respectively, or conservative sequence modifications thereof, and light chain variable region CDR1, CDR2 and CDR3 as set forth in SEQ ID NOs: 50, 51, and 52, respectively, or conservative sequence modifications thereof;
(e) comprising heavy chain variable region CDR1, CDR2 and CDR3 as set forth in SEQ ID NOs: 53, 54, and 55, respectively, or conservative sequence modifications thereof, and light chain variable region CDR1, CDR2 and CDR3 as set forth in SEQ ID NOs: 56, 57, and 58, respectively, or conservative sequence modifications thereof; or
(f) heavy chain variable region CDR1, CDR2 and CDR3 as set forth in SEQ ID NOs: 59, 60, and 61, respectively, or conservative sequence modifications thereof, and light chain variable region CDR1, CDR2 and CDR3 as set forth in SEQ ID NOs: 62, 63, and 64, respectively, or conservative sequence modifications thereof.

70-81. (canceled)

82. A bispecific or multispecific construct comprising the anti-PD-L1 antigen-binding fragment of claim 69 linked to at least one additional binding domain.

83-89. (canceled)

90. A composition comprising the bispecific construct of claim 1 and a pharmaceutically acceptable carrier.

91. A kit comprising the bispecific construct of claim 1 and instructions for use.

92. (canceled)

93. An isolated nucleic acid molecule comprising:
(a) a nucleotide sequence as set forth in SEQ ID NO:25, 26, 27, 28, 101, 102, 103, 104, 105, 106, 107, 108, 109, 110, 111, or 112; or
(b) a nucleotide sequence encoding heavy and/or light chain variable regions of an antibody, wherein the heavy and light chain variable regions comprise the amino acid sequences depicted in SEQ ID NOs: 17 and 18, SEQ ID NOs: 19 and 20, SEQ ID NOs: 77 and 78, SEQ ID NOs: 79 and 80, SEQ ID NOs: 81 and 82, SEQ ID NOs: 83 and 84, SEQ ID NOs: 85 and 86, or SEQ ID NOs: 87 and 88, respectively.

94. (canceled)

95. A nucleic acid molecule coding for the bispecific construct as set forth in claim 1.

96. A nucleic acid molecule as set forth in claim 94 in the form of an expression vector.

97. A nucleic acid molecule as set forth in claim 94 in the form of an expression vector which expresses the bispecific construct when administered to a subject in vivo.

98-99. (canceled)

100. A method for treating a condition or disease in a subject, the method comprising administering to the subject the bispecific construct of claim 1 in an amount effective to treat the condition or disease.

101-107. (canceled)

108. A bispecific tetravalent antibody, comprising:
i) two IgG heavy chains;
ii) two light chains; and
iii) two single chain Fv (scFv) domains;
wherein the two IgG heavy chains and two light chains form an IgG moiety which binds specifically human PD-L1 and wherein the two scFv domains each bind specifically to human CD27, and wherein each scFv domain is connected to the C-terminal residue of one of the IgG heavy chains by a connector sequence.

109-116. (canceled)

117. A composition comprising the bispecific tetravalent antibody of claim 108 and a pharmaceutically acceptable carrier.

118. A kit comprising the bispecific tetravalent antibody of claim 108 and instructions for use.

119. A method of stimulating T cell activity comprising contacting T cells with the bispecific tetravalent antibody of claim 108.

120. A method for inducing or enhancing an immune response in a subject comprising administering to the subject the bispecific tetravalent antibody of claim 108 in an amount effective to induce or enhance an immune response in the subject.

121. A method for treating a condition or disease in a subject, the method comprising administering to the subject the bispecific tetravalent antibody of claim 108 in an amount effective to treat the condition or disease.

122. The method of claim 119, wherein the subject suffers from a condition or disease in which stimulation of an immune response is desired.

123. The method of claim 122, wherein the condition or disease is cancer.

124. The method of claim 123, wherein the cancer is selected from the group consisting of colorectal cancer, ovarian cancer, renal cell carcinoma, head and neck squamous cell carcinoma and glioblastoma.

125. A method of stimulating T cell activity comprising contacting T cells with the antibody, or antigen binding fragment thereof, of claim 58.

126. A method of stimulating T cell activity comprising contacting T cells with the antibody, or antigen binding fragment thereof, of claim 69.

127. A method for inducing or enhancing an immune response in a subject comprising administering to the subject the antibody, or antigen binding fragment thereof, of claim 58 in an amount effective to induce or enhance an immune response in the subject.

128. A method for inducing or enhancing an immune response in a subject comprising administering to the subject the antibody, or antigen binding fragment thereof, of claim 69 in an amount effective to induce or enhance an immune response in the subject.

129. A method for treating a condition or disease in a subject, the method comprising administering to the subject the antibody, or antigen binding fragment thereof, of claim 58 in an amount effective to treat the condition or disease.

130. A method for treating a condition or disease in a subject, the method comprising administering to the subject the antibody, or antigen binding fragment thereof, of claim 69 in an amount effective to treat the condition or disease.
